(19) United States
(12) Patent Application Publication
(10) Pub. No.: US 2024/0277770 A1
(43) Pub. Date: Aug. 22, 2024

(54) SYSTEMS AND METHODS TO IDENTIFY MHC-ASSOCIATED ANTIGENS FOR THERAPEUTIC INTERVENTION

(71) Applicant: GENENTECH, INC., South San Francisco, CA (US)

(72) Inventors: Hem Raj GURUNG, South San Francisco, CA (US); Benjamin Joseph HALEY, South San Francisco, CA (US); Amy Jeane HEIDERSBACH, South San Francisco, CA (US); Juan LI, South San Francisco, CA (US); Christopher Michael ROSE, South San Francisco, CA (US); Ann-Jay TONG, South San Francisco, CA (US); Craig BLANCHETTE, South San Francisco, CA (US); Pamela Pui Fung CHAN, South San Francisco, CA (US); Martine Abraham DARWISH, South San Francisco, CA (US)

(21) Appl. No.: 18/647,560

(22) Filed: Apr. 26, 2024

Related U.S. Application Data
(63) Continuation of application No. PCT/US2022/078831, filed on Oct. 27, 2022.
(60) Provisional application No. 63/409,072, filed on Sep. 22, 2022.
(60) Provisional application No. 63/349,525, filed on Jun. 6, 2022.
(60) Provisional application No. 63/272,933, filed on Oct. 28, 2021.

(51) Int. Cl. A61K 35/17; A61K 39/00; C07K 14/74; C12N 5/0783; C12N 5/09; G01N 33/50

(52) U.S. Cl. CPC A61K 35/17; A61K 39/0011; A61K 39/4611; A61K 39/4631; A61K 39/464414; C07K 14/70539; C12N 5/0636; C12N 5/0694; G01N 33/5044; C12N 2510/00

(57) ABSTRACT

The present disclosure relates to compositions and methods of uses of a monoallelic MHC-expressing cell line, as well as method of identifying a neoepitope-MHIC binding pair and methods of treating a subject having a cancer or tumor expressing a neoantigen and a MHC allele.

### CROSS-REFERENCE TO RELATED APPLICATIONS

This application claims the benefit of priority to U.S. Provisional Application 63/272,933, filed on Oct. 28, 2021; U.S. Provisional Application No. 63/349,525, filed on Jun. 6, 2022; and U.S. Provisional Application No. 63/409,072, filed on Sep. 22, 2022. The entire contents of these applications are incorporated herein by reference in their entireties.

### SEQUENCE LISTING

The instant application contains a Sequence Listing which has been submitted via EFS-Web. The content of the text file named “048893-559001WO_Sequence_Listing.xml”, which was created on Oct. 26, 2022 and is 193,168 bytes in size, is hereby incorporated by reference in its entirety.

### BACKGROUND

The major histocompatibility complex-I (MHCI) is an almost ubiquitously expressed protein complex that is responsible for presenting self- and foreign-derived display peptides on the surfaces of antigen presenting cells to lymphocytes. Display peptide presentation by MHCI is one of the first steps of an adaptive immune response toward destruction of diseased cells or for preservation of healthy cells. The MHCI complex is a non-covalently linked protein heterodimer consisting of a heavy chain (α) and light chain (β2 microglobulin, B2M); in general, the MHCI complex is unstable without a peptide ligand. The display peptide generation pathway enlists the proteasome, which degrades ubiquitinated cytosolic proteins into potential display peptides. These display peptides are subsequently imported into the endoplasmic reticulum where they are further refined, and the active MHCI/display peptide complex is formed via a protein chaperone-assisted process before transport to the cell surface for presentation to cytotoxic, or CD8(+) T cells, to recognize and determine the cellular fate.

The MHCI proteins are encoded by the major histocompatibility complex gene complex, and are also known as members of the human leukocyte antigen (HLA) system. The most common HLA family members are encoded by the HLA-A, HLA-B, and HLA-C loci, although there are approximately 24 total known for the HLA family. Each HLA group contains at least a dozen or more alleles, and differential expression of these alleles leads to a rich diversity of protein outputs (HLA allelic variants). Indeed, there are >20,000 possible different HLA-A, HLA-B, and HLA-C protein complexes, each with their own stability and canonical ligand specificity. The high diversity of the HLA system of proteins enable the system as a whole to recognize a large number of possible antigens, including peptides derived from non-human sources, post-translationally modified self-peptides, and peptides synthesized ex-vivo.

Somatic mutations that arise in tumor tissues, e.g., neoantigens, may be found across multiple patients or unique to an individual's tumor. There is an unmet demand to develop therapeutics against these neoantigens, e.g., by inducing T cell responses through the use of vaccines or engineered T cell therapies.

### SUMMARY

The instant application relates to identifying major histocompatibility class I (MHCI)-associated antigens and related systems, methods, and kits. With the discovered specific binding between antigens and MHCI complexes, which in human consist an HLA protein derived from the polymorphic HLA genes and conserved B2M protein, a subject having a specific HLA allele gene and a tumor or cancer expressing a neoantigen which may be cleaved into a plurality of neoepitopes may be screened out from a plurality of subjects for immunotherapies. For example, cancer vaccines may be designed to encode for neoepitopes that bind within a MHCI (e.g., HLA) complex in the subject, thus inducing immune response in the subject. T cell therapies may also be used to recognize a specific neoepitope-HLA binding pair in a subject.

In one aspect, the present disclosure provides a method of producing a monoallelic MHC-expressing cell line. In some embodiment, the method comprises:
i) obtaining a cell that does not express an endogenous MHC allele;
ii) introducing into the cell a polynucleotide encoding an exogenous MHC allele polypeptide, such that the exogenous MHC allele polypeptide is expressed by the cell to create a monoallelic MHC-expressing cell; and
iii) expanding the monoallelic MHC-expressing cell under conditions to obtain a monoallelic MHC-expressing cell line.

In some embodiments, the cell in step i) was genetically modified to mutate or delete one or more endogenous MHC alleles. In some embodiments, the cell in step i) was genetically modified to mutate or delete an endogenous MHC allele.

In some embodiments, step i) comprises genetically modifying a cell to mutate or delete one or more endogenous MHC alleles. In some embodiments, step i) comprises genetically modifying a cell to mutate or delete an endogenous MHC allele.

In some embodiments, the MHC alleles described herein are MHCI alleles.

In some embodiments, the monoallelic MHC-expressing cell line expresses β2-microglobulin (B2M).

In some embodiments, the MHCI allele is encoded by any one of the following loci: HLA-A, HLA-B, and HLA-C. Exemplary MHCI alleles described herein may include those MHCI alleles known in the art (e.g., HLA alleles in public databases). In some embodiments, the MHCI allele is selected from A*01.01, A*02.01, A*03.01, A*11.01, A*24.02, B*07.02, B*08.01, B*35.01, B*44.02, B*51.01, C*03.04, C*04.01, C*05.01, C*06.02, C*07.01, C*07.02, and C*08.02.

In some embodiments, the method described herein further comprises introducing a polynucleotide cassette encoding a plurality of neoantigen-associated peptides into the monoallelic MHC-expressing cell line. In some embodiments, the plurality of neoantigen-associated peptides are expressed in the monoallelic MHC-expressing cell line and are cleaved into a plurality of neoepitopes, wherein at least one neoepitope specifically binds to the exogenous MHC allele polypeptide. In some embodiments, the at least one neoepitope is 8, 9, 10, 11, 12, or 13 amino acids in length. In some embodiments, the neoantigen-associated peptides were identified by bioinformatics and/or a clinical analysis of tumor mutations. In some embodiments, the at least one of the neoantigen-associated peptides comprises at least 70%, 75%, 80%, 85%, 90%, 91%, 92%, 93%, 94%, 95%, 96%, 97%, 98%, 99%, or more, (plus any value or subrange within the provided ranges, including endpoints) sequence identity to at least one of SEQ ID NOs: 1-72, 75-191, and 195-222. In some embodiments, the at least one of the neoantigen-associated peptides consists of at least one of SEQ ID NOs: 1-72, 75-191, and 195-222. In some embodiments, each of the plurality of neoantigen-associated peptides is between about 20 and about 50 amino acids, between about 25 and about 45 amino acids, between about 30 and about 40 amino acids, between about 20 and about 30 amino acids, between about 30 and about 40 amino acids, between about 40 and about 50 amino acids, plus any value or subrange within the provided ranges (including endpoints), in length.

In some embodiments, the at least one of the neoantigen-associated peptides is selected from the neoantigens listed in the instant specification, sequence listing, and/or Figures.

In some embodiments, the cell described herein is an antigen-presenting cell (APC). In some embodiments, the cell is a HMy2.C1R cell. In some embodiments, the cell is a K562 cell (e.g., ATCC product CCL-243™).

In another aspect, the present disclosure provides a monoallelic MHC-expressing cell line, produced by a method described herein.

In another aspect, the present disclosure provides a system comprising a plurality of monoallelic MHC-expressing cell lines, wherein each cell line does not express an endogenous MHC allele, and wherein each cell line expresses an exogenous MHC allele, such that each cell line expresses a different exogenous MHC allele.

In some embodiments, each of the monoallelic MHC-expressing cell lines was genetically modified to mutate or delete one or more endogenous MHC alleles. In some embodiments, each of the monoallelic MHC-expressing cell lines was genetically modified to mutate or delete an endogenous MHC allele.

In some embodiments, each of the expressed MHC alleles is a MHCI allele.

In some embodiments, the monoallelic MHC-expressing cell line expresses β2-microglobulin (B2M).

Exemplary MHCI alleles described herein may include those MHCI alleles known in the art (e.g., HLA alleles in public databases). In some embodiments, the MHCI allele is encoded by any one of the following loci: HLA-A, HLA-B, and HLA-C. In some embodiments, the MHC allele is selected from A*01.01, A*02.01, A*03.01, A*11.01, A*24.02, B*07.02, B*08.01, B*35.01, B*44.02, B*51.01, C*03.04, C*04.01, C*05.01, C*06.02, C*07.01, C*07.02, and C*08.02.

In some embodiments, each cell line comprises a polynucleotide cassette encoding a plurality of neoantigen-associated peptides. In some embodiments, the plurality of neoantigen-associated peptides are expressed in the plurality of monoallelic MHC-expressing cell line and are cleaved into a plurality of neoepitopes, wherein at least one neoepitope specifically binds to the exogenous MHC allele polypeptide. In some embodiments, the at least one neoepitope is 8, 9, 10, 11, 12 or 13 amino acids in length. In some embodiments, the plurality of neoantigen-associated peptides were identified by bioinformatics and/or a clinical analysis of tumor mutations. In some embodiments, at least one of the plurality of neoantigen-associated peptides comprises at least 70%, 75%, 80%, 85%, 90%, 91%, 92%, 93%, 94%, 95%, 96%, 97%, 98%, 99%, or more, (plus any value or subrange within the provided ranges, including endpoints) sequence identity to at least one of SEQ ID NOs: 1-72, 75-191, and 195-222. In some embodiments, the at least one of the neoantigen-associated peptides consists of at least one of SEQ ID NOs: 1-72, 75-191, and 195-222. In some embodiments, each of the plurality of neoantigen-associated peptides is between about 20 and about 50 amino acids, between about 25 and about 45 amino acids, between about 30 and about 40 amino acids, between about 20 and about 30 amino acids, between about 30 and about 40 amino acids, between about 40 and about 50 amino acids, plus any value or subrange within the provided ranges (including endpoints), in length. In some embodiments, the at least one of the neoantigen-associated peptides is selected from the neoantigens listed in the instant specification, sequence listing, and/or Figures.

In some embodiments, each of the monoallelic MHC-expressing cell lines is an antigen-presenting cell (APC). In some embodiments, the cell is a HMy2.C1R cell. In some embodiments, the cell is a K562 cell (e.g., ATCC product CCL-243™).

In another aspect, the present disclosure provides an isolated polynucleotide cassette encoding a plurality of neoantigen-associated peptides. In some embodiments, each of the plurality of neoantigen-associated peptides is between about 20 and about 50 amino acids, between about 25 and about 45 amino acids, between about 30 and about 40 amino acids, between about 20 and about 30 amino acids, between about 30 and about 40 amino acids, between about 40 and about 50 amino acids, plus any value or subrange within the provided ranges (including endpoints), in length. In some embodiments, the plurality of neoantigen-associated peptides was identified by bioinformatics and/or a clinical analysis of tumor mutations.

In some embodiments, the isolated polynucleotide cassette further comprises at least a linker between two neoantigen-associated peptides. In some embodiments, the linker comprises a peptide linker, such as one of Glycine-Serine (GS) linkers.

In some embodiments, the isolated polynucleotide cassette further comprises at least one promoter capable of initiating translation of the plurality of neoantigen-associated peptides into a single polypeptide in a cell. In some embodiments, the single polypeptide is cleaved into a plurality of neoepitopes in the cell. In some embodiments, the plurality of neoepitopes is presented on the surface of the cell.

In some embodiments, at least one of the plurality of neoantigen-associated peptides comprises at least 70%, 75%, 80%, 85%, 90%, 91%, 92%, 93%, 94%, 95%, 96%, 97%, 98%, 99%, or more, (plus any value or subrange within the provided ranges, including endpoints) sequence identity to at least one of SEQ ID NOs: 1-72, 75-191, and 195-222.

In another aspect, the present disclosure provides a method of identifying a neoepitope-MHC binding pair. In some embodiments, the method comprises:
i) providing a monoallelic MHC-expressing cell line comprising cells, wherein each cell expresses a first exogenous MHC allele and comprises a polynucleotide cassette encoding a plurality of neoantigen-associated peptides;
ii) expressing the plurality of neoantigen-associated peptides in each cell, wherein a plurality of neoepitopes is produced by cleaving the plurality of neoantigen-associated peptides within each cell, such that one or more neoepitopes binds to the first exogenous MHC at the cell surface;
iii) eluting the neoepitope from its bound first exogenous MHC at the cell surface; and
iv) identifying the eluted neoepitope from step iii), thereby identifying a neoepitope-MHC binding pair.

In another aspect, the present disclosure provides a method of identifying a neoepitope-MHC binding pair, comprising:
i) providing a monoallelic MHC-expressing cell line comprising cells, wherein each cell expresses a first exogenous MHC allele;
ii) contacting the cells with a synthesized neoepitope;
iii) eluting peptides bound to the first exogenous MHC allele at the cell surface; and
iv) identifying the eluted neoepitope from step iii), thereby identifying a neoepitope-MHC binding pair.

In some embodiments, the plurality of neoantigen-associated peptides was determined to bind to one or more MHCs by peptide exchange assay.

In some embodiments, the plurality of neoantigen-associated peptides was identified by bioinformatics and/or a clinical analysis of tumor mutations.

In some embodiments, the at least one of the plurality of neoepitopes is 8, 9, 10, 11, 12 or 13 amino acids in length.

In some embodiments, the at least one of the plurality of neoantigen-associated peptides comprises at least 70%, 75%, 80%, 85%, 90%, 91%, 92%, 93%, 94%, 95%, 96%, 97%, 98%, 99%, or more, (plus any value or subrange within the provided ranges, including endpoints) sequence identity to at least one of SEQ ID NOs: 1-72, 75-191, and 195-222.

In some embodiments, each of the plurality of neoantigen-associated peptides is between about 20 and about 50 amino acids, between about 25 and about 45 amino acids, between about 30 and about 40 amino acids, between about 20 and about 30 amino acids, between about 30 and about 40 amino acids, between about 40 and about 50 amino acids, plus any value or subrange within the provided ranges (including endpoints), in length.

In some embodiments, at least one of the plurality of neoantigen-associated peptides is selected from the neoantigens listed in the instant specification, sequence listing, and/or Figures.

In some embodiments, the plurality of neoantigen-associated peptides are expressed in a single polypeptide. In some embodiments, the single polypeptide comprises at least one linker between two neoantigen-associated peptides. In some embodiments, the linker comprises a peptide linker, such as one of Glycine-Serine (GS) linkers.

In some embodiments, the synthesized neoepitope is determined to bind to one or more MHC allele polypeptides by peptide exchange assay.

In some embodiments, steps i) to iv) of the method described herein are repeated in a second monoallelic MHC-expressing cell line comprising cells, wherein each cell expresses a second exogenous MHC allele polypeptide.

In some embodiments, each cell of the monoallelic MHC-expressing cell lines in step i) of the method described herein is genetically modified to mutate or delete an endogenous MHC allele.

In some embodiments, step i) of the method described herein comprises genetically modifying one or more cells of the monoallelic MHC-expressing cell line to mutate or delete one or more (e.g., an) endogenous MHC allele. In some embodiments, the MHC allele is a MHCI allele. In some embodiments, the monoallelic MHC-expressing cell line expresses β2-microglobulin (B2M). Exemplary MHCI alleles described herein may include those MHCI alleles known in the art (e.g., HLA alleles in public databases). In some embodiments, the MHCI allele is encoded by any one of the following loci: HLA-A, HLA-B, and HLA-C. In some embodiments, the MHC allele is selected from A*01.01, A*02.01, A*03.01, A*11.01, A*24.02, B*07.02, B*08.01, B*35.01, B*44.02, B*51.01, C*03.04, C*04.01, C*05.01, C*06.02, C*07.01, C*07.02, and C*08.02.

In another aspect, the present disclosure provides a vaccine, such as a cancer vaccine. In some embodiments, the cancer vaccine comprises an isolated polypeptide or an isolated polynucleotide encoding the polypeptide, wherein the polypeptide comprises a neoepitope in a neoepitope-MHC binding pair identified by a method described herein.

In another aspect, the present disclosure provides a method of preparing a T cell expressing a T cell receptor (TCR) and/or a chimeric antigen receptor (CAR), comprising introducing a TCR and/or a CAR into a T cell, wherein the TCR and/or the CAR specifically binds to a neoepitope-MHC complex, formed by a neoepitope-MHC binding pair identified by a method described herein.

In another aspect, the present disclosure provides a recombinant T cell expressing a T cell receptor (TCR) and/or a chimeric antigen receptor (CAR). In some embodiments, the TCR and/or the CAR specifically binds to a neoepitope-MHC binding pair comprising:
a neoepitope produced by cleaving a neoantigen-associated peptide expressed by a tumor or cancer; and
a MHC allele expressed by the tumor or cancer.

In some embodiments, the MHC allele peptide is a MHCI allele polypeptide.

In some embodiments, the monoallelic MHC-expressing cell line expresses β2-microglobulin (B2M).

Exemplary MHCI alleles described herein may include those MHCI alleles known in the art (e.g., HLA alleles in public databases). In some embodiments, the MHCI allele is encoded by any one of the following loci: HLA-A, HLA-B, and HLA-C. In some embodiments, the MHC allele peptide is encoded by a MHC allele selected from A*01.01, A*02.01, A*03.01, A*11.01, A*24.02, B*07.02, B*08.01, B*35.01, B*44.02, B*51.01, C*03.04, C*04.01, C*05.01, C*06.02, C*07.01, C*07.02, and C*08.02.

In some embodiments, the neoantigen-associated peptide was identified by bioinformatics and/or a clinical analysis of tumor mutations.

In some embodiments, the neoepitope is 8, 9, 10, 11, 12 or 13 amino acids in length.

In some embodiments, the neoantigen-associated peptide comprises at least 70%, 75%, 80%, 85%, 90%, 91%, 92%, 93%, 94%, 95%, 96%, 97%, 98%, 99%, or more, (plus any value or subrange within the provided ranges, including endpoints) sequence identity to at least one of SEQ ID NOs: 1-72, 75-191, and 195-222.

In some embodiments, the neoantigen-associated peptide is between about 20 and about 50 amino acids, between about 25 and about 45 amino acids, between about 30 and about 40 amino acids, between about 20 and about 30 amino acids, between about 30 and about 40 amino acids, between about 40 and about 50 amino acids, plus any value or subrange within the provided ranges (including endpoints), in length.

In another aspect, the present disclosure provides a method of selecting a subject having a cancer or tumor for treatment by a T cell expressing a T cell receptor (TCR) and/or a chimeric antigen receptor (CAR). In some embodiments, the method comprises
(i) genotyping the subject to identify a MHC allele expressed by the subject and a neoantigen expressed by the cancer or tumor, wherein the neoantigen can be cleaved by a cell to produce a plurality of neoepitopes, and
(ii) determining whether the expressed MHC is capable of binding one or more of the neoepitopes, wherein, if the identified MHC and a neoepitope of the plurality of the neoepitopes form a neoepitope-MHC binding pair, the subject is determined as being treatable by the T cell, wherein the TCR and/or the CAR specifically binds to the neoepitope-MHC binding pair.

In another aspect, the present disclosure provides a method of selecting a subject having a cancer or tumor for treatment by a cancer vaccine, comprising
(i) genotyping the subject to identify a MHC protein expressed by the subject and a neoantigen expressed by the cancer or tumor wherein the neoantigen can be cleaved by a cell to produce a plurality of neoepitopes, and
(ii) determining whether the MHC is capable of binding one or more of the neoepitopes, wherein, if the identified MHC protein and a neoepitope of the plurality of the neoepitopes form a neoepitope-MHC binding pair, the subject is determined to be treatable by a cancer vaccine comprising the neoepitope or a polynucleotide encoding the neoepitope.

In another aspect, the present disclosure provides a method of treating a subject having a cancer or tumor expressing a neoantigen-associated peptide and a MHC, wherein the MHC is determined to bind to a neoepitope from the neoantigen, the method comprising administering to the subject a therapeutically effective amount of T cells expressing a T cell receptor (TCR) and/or a chimeric antigen receptor (CAR), wherein the TCR and/or the CAR specifically binds to a neoepitope-MHC binding pair comprising the MHC and the neoepitope.

In another aspect, the present disclosure provides a method of treating a subject having a cancer or tumor. In some embodiments, the method comprises
(i) selecting a subject expressing a MHC allele and having a cancer expressing a neoantigen, wherein the MHC is determined to bind to a neoepitope from the neoantigen, and
(ii) administering to the subject a therapeutically effective amount of T cells expressing a T cell receptor (TCR) and/or a chimeric antigen receptor (CAR), wherein the TCR and/or the CAR specifically binds to a neoepitope-MHC binding pair comprising the MHC and the neoepitope.

In another aspect, the present disclosure provides a method of treating a subject having a cancer or tumor expressing a neoantigen and a MHC allele, wherein the MHC is determined to bind to a neoepitope from the neoantigen, the method comprising administering to the subject a therapeutically effective amount of a vaccine comprising the neoepitope or a polynucleotide encoding the neoepitope.

In some embodiments, the neoepitope is determined to bind to one or more MHCs by peptide exchange assay.

In some embodiments, the neoantigen was identified by bioinformatics and/or a clinical analysis of tumor mutations.

In some embodiments, the neoantigen is a neoantigen listed in the instant specification, sequence listing, and/or Figures.

In some embodiments, the neoepitope is 8, 9, 10, 11, 12 or 13 amino acids in length.

In some embodiments, the neoepitope comprises at least 70%, 75%, 80%, 85%, 90%, 91%, 92%, 93%, 94%, 95%, 96%, 97%, 98%, 99%, or more, (plus any value or subrange within the provided ranges, including endpoints) sequence identity to at least one of SEQ ID NOs: 1-72, 75-191, and 195-222.

In some embodiments, the MHC allele is a MHCI allele.

In some embodiments, the monoallelic MHC-expressing cell line expresses β2-microglobulin (B2M).

Exemplary MHCI alleles described herein may include those MHCI alleles known in the art (e.g., HLA alleles in public databases). In some embodiments, the MHCI allele is encoded by any one of the following loci: HLA-A, HLA-B, and HLA-C. In some embodiments, the MHC allele is selected from A*01.01, A*02.01, A*03.01, A*11.01, A*24.02, B*07.02, B*08.01, B*35.01, B*44.02, B*51.01, C*03.04, C*04.01, C*05.01, C*06.02, C*07.01, C*07.02, and C*08.02.

### BRIEF DESCRIPTION OF THE DRAWINGS

FIGS. 1A-1D illustrate an example of the neoepitope discovery pipeline. FIG. 1A shows clinico-genomics analysis of all known neoantigens, including a summary of shared neoepitope discovery pipeline (the bottom panel). Neoepitope-HLA pairs were tested within a high-throughput binding assay. Binders were further interrogated within engineered cell lines expressing a 47-mer neoantigen cassette by untargeted and targeted immunopeptidomics to confirm presentation of neoepitope-HLA pairs. Selected neoepitopes were then validated for immunogenic potential through discovery of specific TCRs that enable T cell activation and target cell killing. FIG. 1B is a schematic of TR-FRET assay to measure neoepitope-HLA binding and stability. FIG. 1C is a schematic of engineered monoallelic and neoantigen carrying cells. FIG. 1D is a schematic of targeted mass spectrometry to measure neoepitope presentation.

FIGS. 2A-2F are a set of graphs illustrating neoepitope binders identified by TR-FRET and NetMHC. FIG. 2A shows TR-FRET (dots/left axis) and NetMHC (squares/right axis) analysis of KRAS G12R neoepitope binders to the B*07:02 allele. The listed KRAS G12R neoepitopes, from left to right, are defined as SEQ ID NOs: 1-37. FIG. 2B shows TR-FRET (dots/left axis) and NetMHC (squares/right axis) analysis of ESR K303R neoepitope binders to the B*07:02 allele. The listed ESR K303R neoepitopes, from left to right, are defined as SEQ ID NOs: 38-72. FIG. 2C shows percent binders identified in the TR-FRET and NetMHC 4.0 analysis. FIG. 2D compares percent of neoepitope-HLA combinations that were determined to be stable binders by TR-FRET (bars at the right side in each panel) and NetMHC (bars at the left side in each panel) analysis across the HLA A, B and C alleles. FIG. 2E is a graph further illustrating the result in FIG. 2A, showing TR-FRET RZ-score (the bottom panel) and 1/NetMHC Percentile Rank (the top panel) of all neoepitope-HLA combinations for the B*07:02 allele and KRAS G12R neoantigen. The dotted lines in the top and the bottom panels represent the cutoff of stable binders for the NetMHC and TR-FRET analysis, respectively. KRAS G12R neoantigens, from left to right in FIG. 2E, are ARGVGKSA (SEQ ID NO: 5), ARGVGKSAL (SEQ ID NO: 6), ARGVGKSALT (SEQ ID NO: 7), ARGVGKSALTI (SEQ ID NO: 8), EYKLVVVGAR (SEQ ID NO: 35), EYKLVVVGARG (SEQ ID NO: 36), GARGVGKS (SEQ ID NO: 9), GARGVGKSA (SEQ ID NO: 10), GARGVGKSAL (SEQ ID NO: 11), GARGVGKSALT (SEQ ID NO: 12), KLVVVGAR (SEQ ID NO: 28), KLVVVGARG (SEQ ID NO: 29), KLVVVGARGV (SEQ ID NO: 30), KLVVVGARGVG (SEQ ID NO: 31), LVVVGARGV (SEQ ID NO: 25), LVVVGARGVG (SEQ ID NO: 26), LVVVGARGVGK (SEQ ID NO: 27), RGVGKSAL (SEQ ID NO: 1), RGVGKSALT (SEQ ID NO: 2), RGVGKSALTI (SEQ ID NO: 3), RGVGKSALTIQ (SEQ ID NO: 4), TEYKLVVVGAR (SEQ ID NO: 37), VGARGVGK (SEQ ID NO: 13), VGARGVGKS (SEQ ID NO: 14), VGARGVGKSA (SEQ ID NO: 15), VGARGVGKSAL (SEQ ID NO: 16), VVGARGVG (SEQ ID NO: 17), VVGARGVGK (SEQ ID NO: 18), VVGARGVGKS (SEQ ID NO: 19), VVGARGVGKSA (SEQ ID NO: 20), VVVGARGV (SEQ ID NO: 21), VVVGARGVG (SEQ ID NO: 22), VVVGARGVGK (SEQ ID NO: 23), VVVGARGVGKS (SEQ ID NO: 24), YKLVVVGAR (SEQ ID NO: 32), YKLVVVGARG (SEQ ID NO: 33), and YKLVVVGARGV (SEQ ID NO: 34). FIG. 2F is a graph further illustrating the result in FIG. 2B, showing TR-FRET robust Z-score (squares in the bottom panel) and 1/Percentile Rank (dots in the top panel) of all neoepitope-HLA combinations for the B*07:02 allele and ESRI K303R neoantigen. The dotted lines in the top and the bottom panels represent the cutoff of stable binders for the NetMHC and TR-FRET analysis, respectively. ESRI K303R neoantigens, from left to right in FIG. 2F, are IKRSKRNS (SEQ ID NO: 56), IKRSKRNSLA (SEQ ID NO: 57), IKRSKRNSLAL (SEQ ID NO: 58), KRNSLALS (SEQ ID NO: 42), KRNSLALSL (SEQ ID NO: 43), KRNSLALSLT (SEQ ID NO: 44), KRNSLALSLTA (SEQ ID NO: 45), KRSKRNSLA (SEQ ID NO: 53), KRSKRNSLAL (SEQ ID NO: 54), KRSKRNSLALS (SEQ ID NO: 55), LMIKRSKR (SEQ ID NO: 63), LMIKRSKRN (SEQ ID NO: 64), LMIKRSKRNS (SEQ ID NO: 65), LMIKRSKRNSL (SEQ ID NO: 66), MIKRSKRN (SEQ ID NO: 59), MIKRSKRNS (SEQ ID NO: 60), MIKRSKRNSL (SEQ ID NO: 61), MIKRSKRNSLA (SEQ ID NO: 62), PLMIKRSKR (SEQ ID NO: 67), PLMIKRSKRN (SEQ ID NO: 68), PLMIKRSKRNS (SEQ ID NO: 69), PSPLMIKRSKR (SEQ ID NO: 72), RNSLALSL (SEQ ID NO: 38), RNSLALSLT (SEQ ID NO: 39), RNSLALSLTA (SEQ ID NO: 40), RNSLALSLTAD (SEQ ID NO: 41), RSKRNSLAL (SEQ ID NO: 50), RSKRNSLALS (SEQ ID NO: 51), RSKRNSLALSL (SEQ ID NO: 52), SKRNSLAL (SEQ ID NO: 46), SKRNSLALS (SEQ ID NO: 47), SKRNSLALSL (SEQ ID NO: 48), SKRNSLALSLT (SEQ ID NO: 49), SPLMIKRSKR (SEQ ID NO: 70), and SPLMIKRSKRN (SEQ ID NO: 71).

FIGS. 3A-3E are a set of graphs comparing results of TR-FRET and NetMHC analyses. FIG. 3A shows the comparison of the percent agreement between the TR-FRET and NetMHC analysis in identifying binders and non-binders. FIG. 3B shows comparison of the percent agreement between the TR-FRET and NetMHC analysis in identifying binders. FIG. 3C shows comparison of the percent agreement between the TR-FRET and NetMHC analysis in identifying non-binders. FIG. 3D shows comparison of the percent agreement between the TR-FRET and NetMHC analysis in HLA-A, HLA-B, and HLA-C alleles. FIG. 3E shows percent neoepitope-HLA pairs found to be binders by both NetMHCpan 4.0 and TR-FRET across the individual alleles, representing the result of FIG. 3B after correction.

FIGS. 4A-4D are a set of graphs comparing all identified binders between the TR-FRET and NetMHC analyses. FIG. 4A shows a heatmap displaying the number of neoepitope binders for each neoantigen across all HLAs screened for the TR-FRET analysis. FIG. 4B shows a heatmap displaying the number of neoepitope binders for each neoantigen across all HLAs screened for the NetMHC analysis. FIGS. 4C and 4D show heatmaps displaying the number of neoepitope binders for each neoantigen (v-axis) across all 15 HLAs (x-axis) screened for the NetMHC analysis (FIG. 4C) or the TR-FRET analysis (FIG. 4D) from a similar experiment.

FIGS. 5A-5C are a set of graphs illustrating an example of cell engineering process. FIG. 5A shows the structure of an example of piggybac neoantigen cassette containing a concatenated neoantigen expression array, with or without linker between each of neoantigens in the expression array, with control constructs containing several viral peptides known to be presented by A*02:01 at the C-terminus. FIG. 5B shows a process of introducing the piggybac neoantigen expression construct, with or without linker, or control into a K562 cell line stably expressing an A*02:01 allele. HLA IP+LC-MS may be used to detect any neoepitope cleaved from the neoantigens and bound to the expressed A*02:01 allele. FIG. 5C is a graph illustrating some identified viral control peptides on the control constructs, as expected. Illustrated peptides include, ELAGIGILTV (SEQ ID NO: 75), NLVPMVATV (SEQ ID NO: 76), and SLLMWITQV (SEQ ID NO: 214). Those identified by the control set 1 constructs are boxed in rectangles, while those identified by the “all controls” constructs are indicated by arrows.

FIGS. 6A-6H are a set of graphs illustrating an example of cell engineering process. FIG. 6A shows an example of a CRISPR/Cas9 knockout strategy to remove remaining HLA-C allele (HLA-C*04:01) from HMy2.C1R cells, generating an HLA-Class I knockout (Class I KO) HMy2.C1R population useful for producing a monoallelic cell line expressing an exogenous A*02:01. HLA-C*04:01-specific sgRNA (long) contains the sequence of TGGCCCGGCCGCGGGGAGCCCCGCTTCATCGCAGTGGGCTACGTGGACGA (SEQ ID NO: 73), and the HLA-C*04:01-specific sgRNA target sequence contains the sequence of TTCATCGCAGTGGGCTACG (SEQ ID NO: 74). FIG. 6B shows flow cytometric detection of pan-HLA-I expression in wild-type (WT) or HMy2.C1R HLA I-knockout (KO) cells using a pan-HLA-I detection antibody W6/32 or isotype (ISO) control, comparing the differences on HLA expression between such Class I KO cells and the parent wild-type HMy2.C1R. The expression of the exogenous A*02:01 and the piggybac neoantigen expression construct in the Class I KO cells was detected by FACS as shown in FIG. 6C. FIG. 6D illustrates an example of transducing the Class I KO cells to producing cells expressing HLA variants of interest for further analysis. FIG. 6E shows a vector map of the piggyBac polyneoantigen expression constructs utilized in this study. A single transcript containing 47 concatenated neoantigens (about 25 amino acids length for each neoantigen) followed by 7 control peptides and an IRES linked BFP reporter is driven by a pol II Efl alpha promoter. Neoantigens were either directly concatenated (No-Linker) or interspersed by short flexible linker sequences (Linker). FIG. 6F shows flow cytometric detection of HLA-I expression (using W6/32 antibody) and polyantigen cassette expression (BFP, a.k.a., blue fluorescent protein) of selected cell lines or class I knockout parental line. FIG. 6G shows flow cytometric detection of pan-HLA expression (by the W6/32 antibody; the top panels) or expression of a transcriptionally linked TagBFP2 reporter gene (by BFP, the lower panels) in the indicated polyneoantigen-expressing monoallelic cell lines HMy2.C1R^{MHCInull}. FIG. 6H shows targeted immunopeptidomic detection of expression of the control antigen peptides (the left side dark column: NLVPMVATV (SEQ ID NO: 76) originated from pp65; the right side gray column: VLEETSVML (SEQ ID NO: 79) originated from IE-1) in both the linker and no linker HLA-A*02:01-engineered cells.

FIG. 7 is a graph showing counts of 8-11-mer unique peptides per allele for the 17 HLA variants of interest.

FIGS. 8A-8B are a set of graphs illustrating identified neoepitope-HLA binding pairs. FIG. 8A shows an example of 18 neoepitope-HLA binding pairs from 15 shared neoantigens across 4 HLA alleles, identified by the untargeted analysis described herein. Identified neoantigens, represented by from top to bottom blocks, are: NRAS Q61K (ILDTAGKEEY, SEQ ID NO: 125) and NRAS Q61R/HRAS Q61R (ILDTAGREEY, SEQ ID NO: 126) for A*01:01; BRAF V600M (KIGDFGLA™, SEQ ID NO: 112), FGFR3 S249C (YTLDVLERC, SEQ ID NO: 115), FLT3 D835Y (YIMSDSNYV, SEQ ID NO: 116), FLT3 D835Y (YIMSDSNYVV, SEQ ID NO: 117), and TP53 R175H (HMTEVVRHC, SEQ ID NO: 128) for A*02:01; EGFR G719A (ASGAFGTVYK, SEQ ID NO: 113), KRAS G12A (VVVGAAGVGK, SEQ ID NO: 118), KRAS G12C (VVVGACGVGK, SEQ ID NO: 119), KRAS G12D (VVVGADGVGK, SEQ ID NO: 120), KRAS G12S (VVVGASGVGK, SEQ ID NO: 121), KRAS G12V (VVGAVGVGK, SEQ ID NO: 122), KRAS G12V (VVVGAVGVGK, SEQ ID NO: 123), KRAS G13C (VVVGAGCVGK, SEQ ID NO: 124), and TP53 R248Q (SCMGGMNQR, SEQ ID NO: 129) for A*11:01; GFGR3 S249C (ERCPHRPIL, SEQ ID NO: 114) and PIK3CA H1047L (FMKQMNDAL, SEQ ID NO: 127) for B*08:01. FIG. 8B compares NetMHC presentation prediction scores (“EL-mut”) and measured RobustZScores (“RobustzScore”; indicated by arrows or boxed by the oval) for these 18 pairs.

FIG. 9 is a set of graphs illustrating neoepitope-HLA binding pairs identified by untargeted+targeted analyses or target analysis only. NetMHC presentation prediction scores (“EL-mut”, the top panel) and measured RobustZScores (“RobustzScore”, the bottom panel) of these identified pairs are compared.

FIG. 10 is a graph showing scatter plot of TR-FRET RZ-score and Log2 NetMHC percentile rank. The horizontal dashed line represents the cutoff for stable binders as measured by TR-FRET, where values higher than the horizontal dashed line are considered stable binders. The vertical dashed line represents the cutoff for binders based on NetMHC analysis, where values lower than (to the left of) the vertical dashed line are considered binders.

FIG. 11 is a set of graphs illustrating scatter plots similar to the one in FIG. 10, each for one specific allele.

FIGS. 12A-12B are a set of graphs illustrating differences in the protein turnover rate comparing C1R A*11:01 KRAS full-length samples to SharedNeo non-linker sample. For these analyses, A*11:01 monoallelic cells were engineered to express doxycycline (dox)-inducible full-length KRAS wildtype (WT) or mutant proteins (G12C, G12D, G12V). These were compared against an A*11:01 monoallelic cell line containing the no-linker polyantigen cassette. Bars in each panel, from left to right, represent experiments with WT not induced by dox, WT induced by dox, G12C not induced by dox, G12C induced by dox, G12D not induced by dox, G12D induced by dox, G12V not induced by dox, G12V induced by dox, or SharedNeo non-linker control. FIG. 12A shows absolute amount of KRAS WT and the expression of the indicated mutant proteins in the cell lysate by targeted mass spectrometry. Identified peptides include SFEDIHHYR (SEQ ID NO: 175), LVVVGACGVGK (SEQ ID NO: 176), LVVVGADGVGK (SEQ ID NO: 177), and LVVVGAVGVGK (SEQ ID NO: 178). FIG. 12B shows copies per cell of presented KRAS-derived peptides as measured by isotope A*11:01 monomers containing heavy synthetic neoepitope-related peptides spiked in prior to affinity purification and targeted mass spectrometry. NL refers to No Linker. n=1 (NL sample was processed only for the heavy isotope-coded peptide MHC (hipMHC) experiment). Identified peptides include VVGAGGVGK (SEQ ID NO: 179), VVVGAGGVGK (SEQ ID NO: 180), VVGACGVGK (SEQ ID NO: 157), VVVGACGVGK (SEQ ID NO: 119), VVGADGVGK (SEQ ID NO: 160), VVVGADGVGK (SEQ ID NO: 120), VVGAVGVGK (SEQ ID NO: 122), and VVVGAVGVGK (SEQ ID NO: 123).

FIGS. 13A-13D are a set of graphs illustrating an exemplary untargeted immunopeptidomic analysis of monoallelic cell lines expressing the polyantigen cassette. FIG. 13A shows a workflow of an untargeted immunopeptidomic analysis. Illustrated peptide sequences include DARHGGWTT (SEQ ID NO: 181), MKQMNDAR (SEQ ID NO: 182), KICDFGLARY (SEQ ID NO: 183), PIIIGHHAY (SEQ ID NO: 174), VGGLRSERRKW (SEQ ID NO: 184), DILDTAGKEEY (SEQ ID NO: 185), SKITEQEK (SEQ ID NO: 186), ATMKSRWSG (SEQ ID NO: 187), LSEITKQEK (SEQ ID NO: 188), MNDARHGGWT(SEQ ID NO: 189), KQMNDARH (SEQ ID NO: 190), etc. FIG. 13B shows number of unique 8-11-mer peptides for each allele identified in untargeted immunopeptidomic analysis. FIG. 13C shows identified shared cancer neoantigen epitopes. Color represented the log 10 largest area across multiple analyses (arrows show two blocks with the largest values). Identified neoantigens epitopes include, from left to right, KIGDFGLATMK (SEQ ID NO: 133), ASGAFGTVYK (SEQ ID NO: 113), ERCPHRPIL (SEQ ID NO: 114), YIMSDSNYV (SEQ ID NO: 116), YIMSDSNYVV (SEQ ID NO: 117), VVVGAAGVGK (SEQ ID NO: 118), VVVGACGVGK (SEQ ID NO: 119), VVVGADGVGK (SEQ ID NO: 120), VVVGASGVGK (SEQ ID NO: 121), VVGAVGVGK (SEQ ID NO: 122), VVVGAVGVGK (SEQ ID NO: 123), VVVGAGCVGK (SEQ ID NO: 124), ILDTAGKEEY (SEQ ID NO: 125), ILDTAGREEY (SEQ ID NO: 126), ALHGGWTTK (SEQ ID NO: 170), FMKQMNDAL (SEQ ID NO: 127), HMTEVVRHC (SEQ ID NO: 128), and SCMGGMNQR (SEQ ID NO: 129). FIG. 13D compares TR-FRET Robust Z Score (RZ Score) and NetMHC percentile rank (% Rank) score for each epitope identified through untargeted immunopeptidome analysis.

FIG. 14 is a graph showing exemplary number of unique peptides (8-11-mer) identified in untargeted proteomics analysis stratified by allele and linker status of neoantigen construct. Each dot represents a measurement of a cell pellet. Boxes represent the interquartile range and line represents the median.

FIGS. 15A and 15B are a set of graphs illustrating the consistency between binding motif sequences of the presented peptides (FIG. 15B, for experimental motifs) and the corresponding expected motifs (FIG. 15A, for known motifs). Binding motifs were determined by untargeted mass spectrometry. Motifs were generated with GibbsCluster 2.0 with two bins allowing for one bin of the dominant motif and a second bin for non-specific peptides.

FIGS. 16A-16F are a set of graphs illustrating an exemplary targeted immunopeptidomic analysis of monoallelic cell lines expressing a polyantigen cassette. FIG. 16A shows a targeted immunopeptidomic workflow. FIG. 16B shows the number of targeted (the bar at left-side for each allele) and detected (the bar at right-side for each allele) shared cancer neoantigen epitopes for each allele. FIG. 16C compares TR-FRET RZ Score and NetMHC % Rank score for each epitope identified through targeted MS. FIG. 16D shows NetMHC % Rank scores for neoepitopes detected in both untargeted and targeted analyses (left) or only in targeted analysis (right). FIG. 16E shows TR-FRET RZ Score for neoepitopes detected in both untargeted and targeted analyses (left) or only in targeted analysis (right). FIG. 16F shows a summary of neoepitopes-HLA pairs detected from shared cancer neoantigens. Color represented attomol of neoepitopes detected on column during analysis (with arrows showing the blocks having higher Log2 (Attomole) values than others). Bolded squares with centered dots represent neoepitopes also detected in untargeted analysis. The result identifies 84 unique neoepitope-HLA combinations from 37 mutations across 12 alleles. “FRMVDVGGL” (SEQ ID NO: 146) peptide on the heatmap comes from both GNA11 and GNAQ proteins with exact NetMHC EL_mut score of 0.0918. However, this peptide was associated with GNAQ protein (as reflected on the plot) based on Robust Z-score of 8.32 as opposed to 4.42 when associated with GNA11. Similarly, “VDVGGLRSER” (SEQ ID NO: 147) peptide on the heatmap comes from both GNA11 and GNAQ proteins with exact NetMHC EL_mut score of 58.4. However, this peptide was associated with GNAll protein (as reflected on the plot) based on Robust Z-score of 5.00 as opposed to 0.069 when associated with GNAQ. The identified neoepitopes include, from top to bottom, EKSRWSGSHQF (SEQ ID NO: 130), KIGDFGLATEK (SEQ ID NO: 131), IGDFGLATM (SEQ ID NO: 132), KIGDFGLATMK (SEQ ID NO: 133), KMRRKMSP (SEQ ID NO: 134), YTDVSNMSH (SEQ ID NO: 135), YTDVSNMSHLA (SEQ ID NO: 136), MPFGSLLDY (SEQ ID NO: 137), ASGAFGTVY (SEQ ID NO: 138), ASGAFGTVYK (SEQ ID NO: 113), FKKIKVLAS (SEQ ID NO: 139), LASGAFGTVYK (SEQ ID NO: 140), FGRAKLLGA (SEQ ID NO: 141), KITDFGRAK (SEQ ID NO: 222), LTSTVQLIM (SEQ ID NO: 142), STDVGFCTL (SEQ ID NO: 143), KRNSLALSL (SEQ ID NO: 43), MIKRSKRNSL (SEQ ID NO: 61), RSKRNSLAL (SEQ ID NO: 50), SKRNSLAL (SEQ ID NO: 46), CPHRPILQA (SEQ ID NO: 144), ERCPHRPIL (SEQ ID NO: 114), YTLDVLERC (SEQ ID NO: 115), FGLARYIM (SEQ ID NO: 145), YIMSDSNYV (SEQ ID NO: 116), YIMSDSNYVV (SEQ ID NO: 117), FRMVDVGGL (SEQ ID NO: 146), VDVGGLRSER (SEQ ID NO: 147), KPIIIGCH (SEQ ID NO: 148), WVKPIIIGC (SEQ ID NO: 149), IIIGGHAY (SEQ ID NO: 150), HIGHHAY (SEQ ID NO: 151), PIIIGGHAY (SEQ ID NO: 152), VKPIIIGHHAY (SEQ ID NO: 153), SPNGTIQNIL (SEQ ID NO: 154), VVGAAGVGK (SEQ ID NO: 155), VVVGAAGVGK (SEQ ID NO: 118), GACGVGKSAL (SEQ ID NO: 156), VVGACGVGK (SEQ ID NO: 157), VVVGACGVGK (SEQ ID NO: 119), DGVGKSAL (SEQ ID NO: 158), GADGVGKSAL (SEQ ID NO: 159), VVGADGVGK (SEQ ID NO: 160), VVVGADGVGK (SEQ ID NO: 120), GARGVGKSA (SEQ ID NO: 10), GARGVGKSAL (SEQ ID NO: 11), VVVGARGVGK (SEQ ID NO: 23), VVGASGVGK (SEQ ID NO: 161), VVVGASGVGK (SEQ ID NO: 121), GAVGVGKSAL (SEQ ID NO: 162), VVGAVGVGK (SEQ ID NO: 122), VVVGAVGVGK (SEQ ID NO: 123), VVGAGCVGK (SEQ ID NO: 163), VVVGAGCVGK (SEQ ID NO: 124), VVGAGDVGK (SEQ ID NO: 164), RPIPIKYKAM (SEQ ID NO: 165), ILDTAGKEEY (SEQ ID NO: 125), LDTAGKEEY (SEQ ID NO: 166), AGREEYSAM (SEQ ID NO: 167), DTAGREEY (SEQ ID NO: 168), ILDTAGREEY (SEQ ID NO: 126), STRDPLSEITK (SEQ ID NO: 169), ALHGGWTTK (SEQ ID NO: 170), FMKQMNDAL (SEQ ID NO: 127), CNTTARAFAVV (SEQ ID NO: 171), HMTEVVRHC (SEQ ID NO: 128), SCMGGMNQR (SEQ ID NO: 129), GRNSFEVCV (SEQ ID NO: 172), and GRNSFEVHV (SEQ ID NO: 173).

FIG. 17 is a set of graphs comparing absolute amounts of detected neoepitopes for each allele with predicted presentation scores by either Robust Z-score (the bottom panels) or NetMHCpan-4.0 (the top panels) method. Each dot represents a neoeptiope-HLA pair detected within the targeted proteomic analysis. If multiple neoepitope-HLA pairs were detected, the attomole value is the maximum value for that peptide-HLA pair across all analyses.

FIG. 18 is a set of graphs comparing epitope presentation for cell lines containing linker and no liker construct. Each line represents a specific epitope and the slope of the line demonstrates if expression was lower, higher, or similar between monoallelic cell lines containing linker and no-linker polyantigen cassettes.

FIG. 19 is a set of graphs showing analysis of epitope presentation across analysis batches with consideration of presence of linker in construct. Each column represents a specific batch analysis for a cell line expressing polyantigen cassettes with or without linkers. Color represents the absolute abundance measured by the attomole amount detected on column for each neoepitope. The neoepitopes for each HLA are listed, from top to bottom, in the same order as in FIG. 16F.

FIG. 20 is a graph comparing epitope presentation across analysis batches. For each allele, detection of epitopes is displayed for each batch that included analysis of monoallelic cell lines containing that particular allele. Color represents the absolute amount of peptide, in attomole, detected on column for each neoepitope. The neoepitopes are listed, from top to bottom, in the same order as in FIG. 16F.

FIGS. 21A-21M are a set of graphs illustrating functional validation of identified tumor associate neoepitope-HLA pairs. Human CD8+T cells transfected with (A) FLT3-p.D835Y-specific or (B) PIK3CA-p.E545K-specific TCR RNA. Flt3-p.D835Y/HLA-A*02:01-specific TCRs were transfected to primary human CD8+ T cells. The transfected T cells were co-cultured overnight with YIMSDSNYV (SEQ ID NO: 116) peptide-pulsed HLA-A*02:01+T2 cells. FIG. 21A assesses CD137 expression by the transfected T cells at different YIMSDSNYV (SEQ ID NO: 116) peptide concentrations pulsed to the T2 cells. The traces, from top to bottom, represents T cells expressing various FLT3-p.D835Y-specific TCRs (FLT3_TCR_1, FLT3_TCR_2, and FLT3_TCR_3, respectively) and the mock control without TCR expression. T cells were then co-cultured with monoallelic A*02:01 K562 cells expressing either a wild-type FLT3 transgene or a mutant FLT3-p.D835Y transgene. The resulting T cell activation is assessed by the expression of CD137 (FIG. 21B), TNF (pg/mL, FIG. 21C), IFNgamma (pg/mL, FIG. 21D) and GranzymeB (pg/mL, FIG. 21E) by the activated T cells and by killing/lysis of the target K562 cells in FIG. 21F. In FIGS. 21B-21F, bars above each condition of transgene represent, from left to right, mock control without TCR expression, and T cells expressing FLT3_TCR_1, FLT3_TCR_2, or FLT3_TCR_3 (as indicated in FIG. 21B). Similar experiments were performed by transfecting human CD8+ T cells with predicted PIK3CA-p.E545K/HLA-A*11:01 TCRs and mixed with monoallelic HLA-A*11:01 expressing K562 cells incubated with an increasing concentration of the predicted neoepitope, STRDPLSEITK (SEQ ID NO: 169) (only found by targeted MS). FIG. 21G assesses CD137 expression by the transfected T cells at different STRDPLSEITK (SEQ ID NO: 169) peptide concentrations pulsed to the K562 cells. The traces, from top to bottom, represent T cells expressing various PIK3CA-p.E545K-specific TCRs (PIK3CA_TCR_3, PIK3CA_TCR_1, PIK3CA_TCR_2, and PIK3CA_TCR_4, respectively) and the mock control without TCR expression. T cells were then co-cultured with monoallelic HLA-A*11:01 K562 cells expressing either a wild-type PIK3CA transgene or a mutant PIK3CA-p.E545K transgene. The resulting T cell activation is assessed by the expression of CD137 (FIG. 21H), TNF (pg/mL, FIG. 21J), IFNgamma (pg/mL, FIG. 21K) and GranzymeB (pg/mL, FIG. 21L) by the activated T cells and by killing/lysis of the target K562 cells in FIG. 21M. In FIGS. 21H-21M, bars above each condition of transgene represent, from left to right, mock control without TCR expression, and T cells expressing PIK3CA_TCR_1, PIK3CA_TCR_2, PIK3CA_TCR_3, or PIK3CA_TCR_4 (as indicated in FIG. 21H).

### DETAILED DESCRIPTION

After reading this description it will become apparent to one skilled in the art how to implement the present disclosure in various alternative embodiments and alternative applications. However, all the various embodiments of the present invention will not be described herein. It will be understood that the embodiments presented here are presented by way of an example only, and not limitation. As such, this detailed description of various alternative embodiments should not be construed to limit the scope or breadth of the present disclosure as set forth herein.

Before the present technology is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

The detailed description divided into various sections only for the reader's convenience and disclosure found in any section may be combined with that in another section. Titles or subtitles may be used in the specification for the convenience of a reader, which are not intended to influence the scope of the present disclosure.

### I. Definitions

Unless defined otherwise, all technical and scientific terms used herein have the same meaning as commonly understood by one of ordinary skill in the art to which this disclosure belongs. In this specification and in the claims that follow, reference will be made to a number of terms that shall be defined to have the following meanings:

As described herein, any concentration range, percentage range, ratio range or integer range is to be understood to include the value of any integer within the recited range and, when appropriate, fractions thereof (such as one tenth and one hundredth of an integer), unless otherwise indicated.

The terminology used herein is for the purpose of describing particular embodiments only and is not intended to be limiting. As used herein, the singular forms “a”, “an” and “the” are intended to include the plural forms as well, unless the context clearly indicates otherwise.

“Optional” or “optionally” means that the subsequently described event or circumstance can or cannot occur, and that the description includes instances where the event or circumstance occurs and instances where it does not.

The term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” when used with regard to an amount means that the amount may vary by +/−10%.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this disclosure.

As used herein, the term “cancer” refers to all types of cancer, neoplasm or malignant tumors found in mammals (e.g. humans), including leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular]cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

A “sample” or “biological sample” as used herein refers to any specimen intended for analysis. In some embodiments, a sample is taken from a patient. In some embodiments, the sample is a “biological fluid sample.” A “biological fluid sample” as used herein refers to any biological fluid from an organism or subject. Examples include whole blood, plasma, tears, saliva, lymph fluid, urine, serum, cerebral spinal fluid, pleural effusion, and ascites.

The terms “immune response” and the like refer, in the usual and customary sense, to a response by an organism that protects against disease. The response can be mounted by the innate immune system or by the adaptive immune system, as well known in the art.

The terms “modulating immune response” and the like refer to a change in the immune response of a subject as a consequence of administration of an agent, e.g., a compound as disclosed herein, including embodiments thereof. Accordingly, an immune response can be activated or deactivated as a consequence of administration of an agent, e.g., a compound as disclosed herein, including embodiments thereof.

“B Cells” or “B lymphocytes” refer to their standard use in the art. B cells are lymphocytes, a type of white blood cell (leukocyte), that develops into a plasma cell (a “mature B cell”), which produces antibodies. An “immature B cell” is a cell that can develop into a mature B cell. Generally, pro-B cells undergo immunoglobulin heavy chain rearrangement to become pro B pre B cells, and further undergo immunoglobulin light chain rearrangement to become an immature B cells. Immature B cells include T1 and T2 B cells.

“T cells” or “T lymphocytes” as used herein are a type of lymphocyte (a subtype of white blood cell) that plays a central role in cell-mediated immunity. They can be distinguished from other lymphocytes, such as B cells and natural killer cells, by the presence of a T cell receptor on the cell surface. T cells include, for example, natural killer T (NKT) cells, cytotoxic T lymphocytes (CTLs), regulatory T (Treg) cells, and T helper cells. Different types of T cells can be distinguished by use of T cell detection agents.

A “regulatory T cell” or “suppressor T cell” is a lymphocyte which modulates the immune system, maintains tolerance to self-antigens, and prevents autoimmune disease.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may, in embodiments, be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “amino acid side chain” refers to the functional substituent contained on amino acids. For example, an amino acid side chain may be the side chain of a naturally occurring amino acid. Naturally occurring amino acids are those encoded by the genetic code (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine), as well as those amino acids that are later modified, e.g., hydroxyproline, γ-carboxyglutamate, and O-phosphoserine. In embodiments, the amino acid side chain may be a non-natural amino acid side chain.

The term “UV-cleavable amino acid side chain” or “UV-cleavable amino acid” refers to the functional substituent of compounds that have the same basic chemical structure as a naturally occurring amino acid, i.e., an a carbon that is bound to a hydrogen, a carboxyl group, an amino group, and an R group. UV-cleavable amino acids are non-proteinogenic amino acids that either occur naturally or are chemically synthesized. Such analogs may have modified R groups or modified peptide backbones, but retain the same basic chemical structure as a naturally occurring amino acid. UV-cleavable amino acids include, without limitation, 2-nitrophenylglycine (NPG), expanded o-nitrobenzyl linker, o-nitrobenzylcaged phenol, o-nitrobenzyl caged thiol, 32 nitroveratryloxycarbonyl (NVOC) caged aniline, o-nitrobenzyl caged selenides, bis-azobenzene, coumarin, cinnamyl, spiropyran, 2-nitrophenylalanine (2-nF), and 3-amino-3-(2-nitrophenyl)propionic acid (ANP) amino acid analogs.

The term “MHC” or “major histocompatibility complex” as provided herein includes a large locus on vertebrate DNA containing a set of closely linked polymorphic genes that code for cell surface proteins essential for the adaptive immune system. These cell surface proteins are MHC molecules. In the instant application, MHC may refer to either DNA or polynucleotides containing the related genes, or the proteins or polypeptides encoded by the related DNA or polynucleotides.

The term “MHCI” or “major histocompatibility complex class I” or “major histocompatibility complex I” or “MHCI monomer” as provided herein includes any of the recombinant or naturally-occurring forms of the major histocompatibility complex-1 (MHCI) protein, or variants, paralogs, or homologs thereof that maintain MHCI activity (e.g. having at least 50%, 60%, 70%, 80%, 90%, 95%, 96%, 97%, 98%, 99%, 100%, or more, activity compared to MHCI). In some aspects, the variants, paralogs, or homologs have at least 50%, 60%, 70%, 80%, 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring MHCI polypeptide. In embodiments, MHCI is a heterodimer of two non-covalently bound proteins, a heavy chain (α) and a light chain (B2-microglobulin), homolog or functional fragment thereof. In embodiments, MHCI includes a peptide ligand. In some embodiments, the term “MHCI” or “major histocompatibility complex class I” may also refer to the DNA or polynucleotides encoding the recombinant or naturally-occurring forms of the MHCI protein described herein.

The term “HLA” or “human leukocyte antigen” refers to a group of proteins encoded by the MHC gene complex, or a group of DNA or polynucleotides containing the genes on human chromosome 6, which encode such proteins. In some embodiments, the MHC gene complex encodes for the HLA-A, HLA-B, and HLA-C group of proteins.

The term “Beta-2 microglobulin” or “B2M” or “β2 microglobulin” or “beta chain” refers to the invariant smaller, or light chain protein of the cell surface MHCI or HLA protein complex. B2M forms a heterodimeric complex with one a chain (heavy chain). B2M is encoded by the B2M gene.

The term “a chain” or “alpha chain” refers to the larger, or heavy chain protein of the MHCI or HLA protein complex. The a chain is further divided into subunits a1, a2, and a3, and contains one transmembrane helix. The a chain binds B2M via the a3 subunit to form the heterodimer known as the MHCI or HLA complex. The a chain is polymorphic, and encoded by mainly the HLA-A, HLA-B, and HLA-C genes, and to a lesser extent by HLA-E, HLA-F, HLA-G, HLA-K, and HLA-L.

As used herein, the term “antigen” is used to describe a compound, composition, or chemical that induces an immune response, e.g., cytotoxic T lymphocyte (CTL) response, a B cell response (for example, production of antibodies that specifically bind the epitope), an NK cell response or any combinations thereof, when administered to an immunocompetent subject. Thus, an immunogenic or antigenic composition is a composition capable of eliciting an immune response in an immunocompetent subject.

As used herein, the term “neoantigen” or “neoantigen-associated peptide” is used to describe newly formed antigens or antigen-associated peptides that may be recognized by the host's immune system as “non-self.” Neoantigens can arise from altered tumor proteins formed as a result of tumor mutations, or bacteria or other pathogens (e.g., from viral proteins). Or they may also be derived from grafts such as tissue grafts or allografts or other transplanted cells. Non-limiting examples of neoantigens are listed in Table 2 below or elsewhere in the instant specification and figures.

As used herein, the terms “tumor associated antigen” or “TAA” are used to describe proteins that are significantly over-expressed in cancer compared to normal cells, and are therefore also abundantly presented on the cancer cell's surface. Non-limiting examples of TAAs are listed in Table 1 below or elsewhere in the instant specification and figures.

TABLE 1
Illustrative Tumor-Associated Antigens

| | | | | |
|---|---|---|---|---|
| WT1 | MAGEA1 | MSLN | PRAME | CTAG1A (NY-ESO) |

TABLE 2
Illustrative Shared Neoantigens

| | | | | |
|---|---|---|---|---|
| JAK2 V617F | PIK3CA H1047R | IDH2 R140Q | KRAS G13D | MYD88 L265P |
| BRAF V600E | EGFR L858R | FLT3 D835Y | NRAS Q61R | DNMT3A R882H |
| BRAF V600M | EGFR E746_A750del | ERBB2 S310F | NRAS Q61K | IDH1 R132H |
| KRAS G12V | TP53 R175H | FGFR3 S249C | PIK3CA E542K | SF3B1 R625C |
| KRAS G12C | TP53 R248Q | PTEN R130Q | PIK3CA E545K | GTF2I L424H |
| KRAS G12D | TP53 R273C | PTEN R130G | TP53 R273L | GNAQ Q209P |
| KRAS G12R | TP53 R273H | SF3B1 R625H | TP53 R282W | GNAQ Q209L |
| | | | | GNA11 Q209L |

The terms “bind” and “bound” as used herein are used in accordance with their plain and ordinary meaning and refer to the association between atoms or molecules. The association can be direct or indirect. For example, bound atoms or molecules may be direct, e.g., by covalent bond or linker (e.g. a first linker or second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like).

The term “antibody” refers to a polypeptide encoded by an immunoglobulin gene or functional fragments thereof that specifically binds and recognizes an antigen. Immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10- to 100-times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

The term “denature” refers to a process where the three-dimensional structure of a protein, polypeptide, DNA, RNA, or other biopolymer is disrupted by chemical or mechanical means, or by heating or cooling.

The process of “peptide-exchange” refers to first forming a MHCI or HLA complex bound to a peptide that is capable of being replaced by, or exchanged for, another peptide of analytical interest such as a putative neoantigen-associated peptide. In some cases, exchange can be promoted by decreasing the binding affinity of the first peptide for the peptide of interest, such as through chemical, enzymatic, or UV-mediated cleavage of the first peptide.

A “ID-LC” or “one-dimensional liquid chromatography” process refers to a single liquid chromatography separation, in contrast to a “2D-LC” or “two-dimensional liquid chromatography,” which refers to a method of chromatography in which two separations are performed.

“Size exclusion chromatography” or “SEC,” is a means of chromatography in which molecules are separated by size on a solid phase chromatography medium, with larger molecules travelling through the solid phase column at a different rate than smaller molecules. In some embodiments, SEC is used in a ID-LC process. SEC could also be used in a 2D-LC process combined with a different form of separation, such as a reversed-phase liquid chromatography step, or an ion exchange or cation exchange or affinity separation, may be employed as a second dimension.

“Capillary electrophoresis” or “CE” refers to a process in which an electric current is used to move molecules through a capillary. Each molecule's mobility may depend on its charge, size, and shape. There are several types of CE, including capillary zone electrophoresis (CZE), capillary gel electrophoresis (CGE), micellar electrokinetic capillary chromatography (MEKC), capillary electrochromatography (CEC), capillary isoelectric focusing (CIEF), and capillary isoelectrophoresis (CITP), among others.

“Capillary zone electrophoresis” or “CZE” as used herein refers to a type of CE in which different molecules in a buffer solution can be separated based on their different mobilities.

“Mass spectrometry” or “MS” refers to a technique that measures the mass to charge ratio (m/z) of one or more molecules in a sample. As used herein, “tandem MS” or “MS/MS” refers to the process by which a single ion, multiple ions, or the entire mass envelope (the precursor(s)) are moved to a fragmentation chamber and the fragmented products are then sent to a mass analyzer. Depending on the design of the mass spectrometer, the fragmentation event can happen before a single mass analyzer, between two or multiple different analyzers, or within a single mass analyzer.

MS analysis may have a variety of options. In some embodiments, the MS instrument does not comprise a quadrupole. In some embodiments, the MS instrument comprises at least one quadrupole. In some embodiments, the MS instrument comprises at least 2 quadrupole analyzers. In some cases, the MS instrument comprises an octopole. In some embodiments, the MS instrument comprises at least 3 quadrupole analyzers. In some MS's, the detector is an ion trap, quadrupole, orbitrap, or TOF. In some embodiments, the MS instrument or method is multiple reaction monitoring (MRM), single ion monitoring (SIM), triple stage quadrupole (TSQ), quadrupole/time of flight (QTOF), quadrupole linear ion trap (QTRAP), hybrid ion trap/FTMS, time of flight/time of flight (TOF/TOF), Orbitrap instruments, ion trap instruments, parallel reaction monitoring (PRM), data dependent acquisition (DDA), data independent acquisition (DIA), multi-stage fragmentation or tandem in time MS/MS. In some embodiments, an electrospray, Orbitrap instrument is used.

“Native mass spectrometry” is an MS process that is performed on a molecule in its native state, i.e., wherein the molecule is not unfolded or denatured.

As used herein, the abbreviation “SEC-MS” refers to an SEC followed by/coupled with a mass spectrometry (MS) process. As used herein, the abbreviations “CE-MS” and “CZE-MS”refer to a capillary electrophoresis (CE) or capillary zone electrophoresis (CZE) followed by a MS process. “SEC-native MS” refers to an SEC followed by/coupled with a native MS process. “CE-native MS” and “CZE-native MS” refer to a capillary electrophoresis (CE) or capillary zone electrophoresis (CZE) followed by/coupled with a native MS process.

The term “quantitation” or “quantitate” means herein to determine numerically the level or amount or number or concentration of an analyte in the sample.

In general, a “subject” as referred to herein is an individual whose biological sample is to be tested for presence of an analyte, and/or who is to be evaluated and/or treated for a disease. In some embodiments, the subject is a human. However, in some embodiments, the subject may also be another mammal, such as a domestic or livestock species, e.g., dog, cat, rabbit, horse, pig, cow, goat, sheep, etc., or a laboratory animal, such as a mouse or rat. Mammals include, but are not limited to, domesticated animals (e.g., cows, sheep, cats, dogs, and horses), primates (e.g., humans and non-human primates such as monkeys), rabbits, and rodents (e.g., mice and rats), for example.

As used herein, an “automated” or “automatically controlled” process is one that is capable of being run, for example, by a computerized control system with appropriate software, as opposed to a system that requires an active, manual intervention during or between at least one step, such as to move an analyte-containing sample from one part of the system to another.

### II. Neoantigens and neoepitopes

The presentation of neoantigen-associated peptides to cytotoxic T lymphocytes is central to eliciting anti-tumor immune responses. However, the identification of these neoantigen-associated peptides in the right HLA context remains a challenge. The instant disclosure relates to an immunopeptidomics pipeline to identify clinically relevant neoantigens in a wide range of HLA alleles. Candidate shared neoantigens may be selected based on unmet clinical need, while their peptide-HLA binding affinities may be predicted using a predictive process, e.g. artificial neural networks such as NetMHCpan4.0. Experimental binding data are then acquired with a novel high-throughput in vitro binding assay. The predicted binding affinities of the neoantigen-associated peptides to the HLA proteins of interest are highly correlated with the resulting experimental binding affinities found through the high-throughput binding assay. To enable detection of neoepitopes by mass spectrometry, a CRISPR/Cas9 engineered HLA deficient antigen presenting cell line (e.g., HMy2.C1R) may be electroporated with minigenes containing one or several shared cancer neoantigens followed by transduction of HLAs of interest. Then engineered monoallelic cell lines containing the neoantigen minigenes may be processed for automated pan-Class-I HLA enrichment and subsequent data-dependent and targeted mass spectrometry assays. Pan-HLA Class I enrichment of the cell lines can produce hundreds or thousands peptides with matching motifs, such as ˜ 850-7300 unique 8- to 11-mer (8-, 9-, 10-, and 11-mer). Targeted mass spectrometry assays identified previously identified neoantigen-HLA combinations as well as many novel combinations. Interestingly, unique neoantigen HLA combinations were detected to have unfavorable NetMHC binding scores but were assayed based on in vitro binding assay performance. These data demonstrate that engineered monoallelic cells expressing neoantigens of interest are an excellent model system to study neoantigen presentation. The current neoantigen prediction algorithms and the binding assays have inherent limitations which can be overcome by using both prediction and the high-throughput (HTP) binding assay in conjunction. Finally, the immunogenicity of select hits identified in the assays were validated through in vitro T cell activation and T cell-directed tumor killing assays.

In some aspects, the instant disclosure relates to identifying neoepitopes (e.g., peptides cleaved from a single or plurality of neoantigens expressed by a cancer or tumor) capable of specifically binding to major histocompatibility class I (MHCI) peptides (e.g., HLA peptides) to form a neoepitope-HLA complex for presenting the neoepitope as a foreign antigen on the surface of the HLA-expressing cell. In some embodiments, immune cells (such as T cells, either engineered or native) expressing corresponding T cell receptors (TCRs) or chimeric antigen receptor (CARs) may recognize the presented foreign antigen by binding to the HLA-neoepitope complex, while such recognition may lead to T cell activation and proliferation to produce anti-cancer cytokines or exert cytotoxicity to kill or inhibit growth of the cancer or tumor.

In some embodiments, neoantigens described herein refer to newly formed antigens or peptides that have not been previously recognized by the immune system. Neoantigens can arise from altered tumor proteins formed as a result of tumor mutations, or bacteria or other pathogens (e.g., from viral proteins). They may also be derived from grafts such as tissue grafts or allografts or other transplanted cells. Non-limiting examples of neoantigens are listed in Table 2.

In some embodiments, a neoantigen described herein comprises a peptide expressed by a tumor or cancer cell in a subject. In some embodiments, the neoantigen-associated peptide comprises at least one mutation (including, e.g., substitution, deletion, insertion, cross-linking, etc.) to at least one amino acid residue, compared to the sequence of the corresponding wild-type peptide expressed by a wild-type cell, a healthy cell, or a non-tumor or non-cancer cell. The difference between the sequence of the neoantigen-associated peptide and the wild-type peptide, e.g., caused by the at least one mutation, may render the neoantigen-associated peptide “not previously recognized” by the immune system in a subject and, thus, capable of inducing immune response in the subject. In some embodiments, a neoantigen described herein is between about 5 and about 100, about 5 and about 90, about 5 and about 80, about 5 and about 70, about 5 and about 60, about 5 and about 50, about 5 and about 40, about 5 and about 30, about 5 and about 20, about 10 and about 100, about 10 and about 90, about 10 and about 80, about 10 and about 70, about 10 and about 60, about 10 and about 50, about 10 and about 40, about 10 and about 30, about 10 and about 20, about 20 and about 90, about 20 and about 80, about 20 and about 70, about 20 and about 60, about 20 and about 50, about 20 and about 40, or about 20 and about 30 (and all sub-values and sub-ranges there between, including endpoints) amino acids in length. In some embodiments, a neoantigen described herein is between about 20 and about 50 amino acids in length.

In some embodiments, a neoantigen described herein is expressed by a tumor or cancer cell in a subject and further cleaved inside the tumor or cancer cell. Such cleavage may be induced by various proteinases or through the proteasome system. In some embodiments, a plurality of peptides may be produced by the cleavage of the neoantigen. For example, a plurality of peptides containing the at least one mutation differentiating the neoantigen from the corresponding wild-type peptide may be produced by the cleavage. In some embodiments, these plurality of peptides are then secreted out of the cell and presented by the expressed HLA allele peptides on the cell surface for recognition by immune cells (e.g., T cells). Among the plurality of peptides, those with a potential capability to activate immune cells, upon recognition of the presented peptides, and thus induce immune response in the subject, may be determined as neoepitopes.

In some embodiments, a neoepitope described herein is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acids in length. In some embodiments, a neoepitope described herein is about 8, 9, 10, 11, 12 or 13 amino acids in length. In some embodiments, a neoepitope described herein is between 8 and 13 amino acids in length. In some embodiments, a neoepitope described herein is between 8 and 11 amino acids in length.

Examples of neoantigen and neoepitope sequences may be found in the instant specification and figures.

Neoantigens and neoepitopes described herein may be specific to the tumor or cancer in the subject. In some embodiments, a plurality of subjects may have a same neoantigen and/or a same plurality of neoepitopes (the “shared neoantigen/neoepitope” scenario). In some embodiments, a subject may have a specific neoantigen and/or a specific plurality of neoepitopes (e.g., at least one neoantigen or neoepitope different from the ones in other subjects) (the “personalized neoantigen/neoepitope” scenario). One aspect of the instant disclosure is about identification of specific MHC (e.g., MHCI, or HLA) alleles expressed by an immune cell which specifically recognize the neoepitope-HLA binding pair in a single or plurality of subjects. Such information is useful for designing cancer/tumor vaccines or T cell therapies in a subject expressing a specific neoantigen and/or neoepitope.

In some embodiments, a specific HLA allele is known (e.g., by genotyping a subject) and neoepitopes specifically binding to the HLA protein are to be discovered. In some embodiments, a peptide exchange assay may be used to identify such neoepitopes. For example, a plurality of neoepitopes, cleavage by a single or a plurality of neoantigens, may be used for the peptide exchange assay. To prepare the plurality of neoepitopes, a single or a plurality of neoantigens expressed by a cancer or tumor cell in the subject having the specific HLA allele may be randomly combined and cleaved to produce the plurality of neoepitopes (“untargeted”). Alternatively, various analyses (e.g., bioinformatics analysis, clinical analysis, etc.) may be done to screen out neoantigens and/or neoepitopes of interest or indication of possible role in the cancer/tumor prevalence and/or clinical value to prepare the plurality of neoepitopes for the peptide exchange assay (“targeted”).

### Peptide Exchange Assay

In some aspects, the instant disclosure provides a peptide exchange assay for determining binding of a major histocompatibility complex class I (MHCI) allele to a test peptide, including: providing a first mixture, containing a free test peptide and a MHCI/ligand (e.g., HLA/ligand) complex that contains an alpha chain, a beta chain, and peptide ligand that contains a non-natural, ultraviolet (UV)-cleavable amino acid within its sequence; exposing the first mixture to UV light to cleave the peptide ligand at the UV-cleavable amino acid; and incubating the first mixture for a period of time to form a second mixture, containing a second MHCI (e.g., HLA) complex that contains the alpha chain, the beta chain, and the test peptide; and determining whether the MHCI (e.g., HLA) allele is bound to the test peptide.

In embodiments, the amount of free test peptide in the first mixture is 1:100 to 100:1 compared to the HLA/ligand complex. In some embodiments, the amount of free test peptide in the first mixture is 1:10 to 10:1 compared to the HLA/ligand complex. In embodiments, the amount of free test peptide in the first mixture is 1:1 to 100: 1 compared to the HLA/ligand complex. In embodiments, the amount of free test peptide in the first mixture is 10:1 to 100:1 compared to the HLA/ligand complex. In embodiments, the amount of free test peptide in the first mixture is about 10:1 compared to the HLA/ligand complex. The ratio may be any value or subrange within the provided ranges, including endpoints.

In embodiments, the HLA binding to the test peptide is determined by measuring a level of HLA/test peptide complex in the second mixture. In some embodiments, the HLA complex in the assay is partially occupied by bound test peptide in the second mixture (a portion of the total HLA complexes in the second mixture is bound by the test peptide). In some embodiments, the HLA complex is fully occupied by bound test peptide in the second mixture (all of the total HLA complexes in the second mixture is bound by the test peptide).

In embodiments, the level of HLA/second peptide complex is measured by 2-dimensional liquid chromatography-mass spectrometry (2D LC/MS) of the second mixture. In some embodiments, the 2D LC/MS includes removing the free test peptide from the second mixture. In some embodiments, the free test peptide is removed by size-exclusion chromatography. In some embodiments, the free test peptide is removed by size cut-off filtration. In some embodiments, the free peptide is removed by dialysis.

In embodiments, high-performance liquid chromatography (HPLC) and mass spectrometry (MS) to distinguish the identities of the HLA and the test peptide. In some embodiments, the second mixture is run over an HPLC (or FPLC) equipped with a size-exclusion column. In some embodiments, the HPLC (or FPLC) is equipped to collect fractions. In some embodiments, the HLA and test peptide are identified to elute in the same HPLC fraction. In some embodiments, free test peptide elutes in fractions different from free HLA and HLA/test peptide complex. In some embodiments, the co-elution of HLA and test peptide indicates that the HLA is capable of binding the test peptide.

In embodiments, there is more than one test peptide (e.g., more than one peptide sequence) added to the first HLA/test peptide mixture. In some embodiments, there are two or more test peptides in the first HLA/test peptide mixture. In embodiments, there are three or more test peptides in the first HLA/test peptide mixture. In embodiments, there are four or more test peptides in the first HLA/test peptide mixture. In embodiments, there are five or more test peptides in the first HLA/test peptide mixture. In embodiments, there are six or more test peptides in the first HLA/test peptide mixture. In embodiments, there are seven or more test peptides in the first HLA/test peptide mixture. In embodiments, there are eight or more test peptides in the first HLA/test peptide mixture. In embodiments, there are nine or more test peptides in the first HLA/test peptide mixture. In embodiments, there are ten or more test peptides in the first HLA/test peptide mixture.

In embodiments, there are 10-1000 test peptides in the first HLA/test peptide mixture. In embodiments, there are 10-500 test peptides in the first HLA/test peptide mixture. In embodiments, there are 10-200 test peptides in the first HLA/test peptide mixture. In embodiments, there are 10-20 test peptides in the first HLA/test peptide mixture. In embodiments, there are 20-30 test peptides in the first HLA/test peptide mixture. In embodiments, there are 30-40 test peptides in the first HLA/test peptide mixture. In embodiments, there are 40-50 test peptides in the first HLA/test peptide mixture. In embodiments, there are 50-60 test peptides in the first HLA/test peptide mixture. In embodiments, there are 60-70 test peptides in the first HLA/test peptide mixture. In embodiments, there are 70-80 test peptides in the first HLA/test peptide mixture. In embodiments, there are 80-90 test peptides in the first HLA/test peptide mixture. In embodiments, there are 90-100 test peptides in the first HLA/test peptide mixture. In embodiments, there are 100-110 test peptides in the first HLA/test peptide mixture. In embodiments, there are 110-120 test peptides in the first HLA/test peptide mixture. In embodiments, there are 120-130 test peptides in the first HLA/test peptide mixture. In embodiments, there are 130-140 test peptides in the first HLA/test peptide mixture. In embodiments, there are 140-150 test peptides in the first HLA/test peptide mixture. In embodiments, there are 150-200 test peptides in the first HLA/test peptide mixture. In embodiments, there are 200-300 test peptides in the first HLA/test peptide mixture. In embodiments, there are 300-400 test peptides in the first HLA/test peptide mixture. In embodiments, there are 400-500 test peptides in the first HLA/test peptide mixture. In embodiments, there are 500-600 test peptides in the first HLA/test peptide mixture. In embodiments, there are 600-700 test peptides in the first HLA/test peptide mixture. In embodiments, there are 700-800 test peptides in the first HLA/test peptide mixture. In embodiments, there are 800-900 test peptides in the first HLA/test t peptide mixture. In embodiments, there are 900-1000 test peptides in the first HLA/test peptide mixture. The number of test peptides may be any value or subrange within the provided ranges, including endpoints. The number of test peptides are only limited by the number of test peptides recognized as feasible to use in the exchange assay by one skilled in the art.

In embodiments, there is more than one test peptide co-eluting in the HLA/peptide complex HPLC fraction.

In embodiments, mass spectrometry is used to identify the identities of HLA and/or test peptide(s) in the second mixture. In some embodiments, the mass spectrometer is in-line with the HPLC. In some embodiments, the HPLC fractions are collected first, then analyzed by mass spectrometry. In embodiments, the free test peptide is removed before mass spectroscopic detection of the HLA complex. In embodiments, the amount of test peptide present in a fraction or in the second mixture is quantified by mass spectrometry by comparison to an internal standard peptide.

In embodiments, the HLA/test peptide complex is labeled. In some embodiments, the HLA/test peptide is fluorescently labeled. In some embodiments, the HLA/test peptide complex is labeled by contacting a fluorescently labeled antibody. In some embodiments, the HLA/test peptide complex is labeled by contacting a fluorescent antibody, where the fluorescent antibody is anti-HLA. In some embodiments, the HLA/peptide complex is labeled by biotinylation of the alpha protein.

In embodiments, the level of peptide exchange is determined by contacting the labeled HLA/peptide complex with an antibody complex containing anti-HLA antibody covalently attached to a fluorescence resonance energy transfer (FRET) donor; and a FRET acceptor complex comprising a FRET acceptor conjugated to a second label, thereby forming a reaction composition; and detecting FRET emission of the second label in the reaction composition, thereby detecting formation of a stable HLA, which is a proxy measure of peptide binding. In some embodiments, the first label is an anti-HLA antibody that is anti-B2M. In some embodiments, the first label is an anti-HLA antibody that is chelating a Europium ion. In some embodiments, the alpha protein of the HLA/peptide complex is biotinylated. In some embodiments, the biotinylated HLA/peptide complex binds a second label. In some embodiments, the second label is a streptavidin protein. In some embodiments, the second label is a streptavidin protein that is covalently linked to an allophycocyanin. In some embodiments, the first and second labels have a spectral overlap integral suitable for FRET when a HLA/peptide complex containing a first and second label is present. In some embodiments, the FRET donor/acceptor pair labels include fluorescein and tetramethylrhodamine. In some embodiments, the FRET donor/acceptor pair labels include 5-({2-[(iodoacetyl)amino]ethyl} amino)naphthalene-1 sulfonic acid (IAEDANS) and fluorescein. In some embodiments the FRET donor/acceptor pair labels include (5-((2-aminoethyl)amino)naphthalene-1-sulfonic acid (EDANS) and 4-((4-(dimethylamino)phenyl)azo)benzoic acid (Dabcyl). In some embodiments, the FRET donor/acceptor pair labels include Alexa Fluor 488 and Alexa Fluor 555. In some embodiments, the donor/acceptor pair labels include Alexa Fluor 594 and Alexa Fluor 647. In some embodiments, the donor/acceptor pair labels include europium (Eu-cryptate) and allophycocyanin (XL665). In some embodiments, the donor/acceptor pair labels include terbium and fluorescein. The first and second labels may be any suitable label pairs known in the art.

In embodiments, the peptide exchange detection assay reagents and the HLA/peptide complex are incubated for between about 1 hour and about 48 hours. In embodiments, the peptide exchange detection assay reagents and the HLA/peptide complex are incubated for at least about 1 hour. In some embodiments, the peptide exchange detection assay reagents and the HLA/peptide complex are incubated for at least about 5 hours. In embodiments, the peptide exchange detection assay reagents and the HLA/peptide complex are incubated for at least about 10 hours. In embodiments, the peptide exchange detection assay reagents and the HLA/peptide complex are incubated for at least about 12 hours. In some embodiments, the peptide exchange detection assay reagents and the HLA/peptide complex are incubated for at least about 15 hours. In some embodiments, the peptide exchange detection assay reagents and the HLA/peptide complex are incubated for at least about 20 hours. In some embodiments, the peptide exchange detection assay reagents and the HLA/peptide complex are incubated for at least about 24 hours. Incubation time may be any value or subrange within the provided ranges, including endpoints.

In some embodiments, the first label contains a streptavidin protein. In some embodiments, the first label contains an anti-HLA antibody. In some embodiments, the first label contains a monobody. In some embodiments, the first label contains a partial antibody. In some embodiments, the first label contains an scFv domain. In some embodiments, the first label contains an antibody fragment.

In some embodiments, the second label is streptavidin.

In embodiments, emission from the FRET acceptor indicates binding of a test peptide to the HLA complex. In some embodiments, the level of bound peptide is determined by time resolved (TR) FRET detection. In some embodiments, the signal from the TR-FRET acceptor label indicates the level of HLA complex present. In some embodiments, the level of HLA complex present indicates the presence of a HLA/peptide complex. In some embodiments, the HLA/peptide complex contains a test peptide. In some embodiments, the signal from FRET emission is normalized between two or more HLAs. In some embodiments, the TR-FRET assay is performed at a temperature between about 4° C. and about 50° C. In some embodiments, the TR-FRET assay is performed at room temperature. In some embodiments, the TR-FRET assay is performed at about 37° C.

Peptide exchange assays and FRET assays are also described in PCT Application No. PCT/US21/47537, filed Aug. 25, 2021 and published as WO 2022/046895, which is incorporated herein by reference for everything taught therein, including, without limitation, all methods, reagents, neoantigens, neoepitopes, examples, systems, etc.

### III. MHC alleles

In some aspects, the instant disclosure relates to identifying neoepitopes (e.g., peptides cleaved from a single or plurality of neoantigens expressed by a cancer or tumor) capable of specifically binding to major histocompatibility class I (MHCI) peptides (e.g., HLA peptides) to form a neoepitope-MHCI complex (a neoepitope-HLA complex in human) for presenting the neoepitope as a foreign antigen on the surface of the MHCI-expressing cell. In some embodiments, immune cells (such as T cells, either engineered or native) expressing corresponding T cell receptors (TCRs) or chimeric antigen receptor (CARs) may recognize the presented foreign antigen (i.e., the neoepitope) by binding to the MHCI-neoepitope complex, while such recognition may lead to T cell activation and proliferation to produce anti-cancer cytokines or exert cytotoxicity to kill or inhibit growth of the cancer or tumor.

In embodiments, the HLA/ligand complex contains an alpha chain, where the alpha chain is encoded by any one of the following loci: HLA-A, HLA-B, and HLA-C. In some embodiments, the alpha chain is encoded by the HLA-A loci. In some embodiments, the alpha chain is encoded by the HLA-B loci. In some embodiments, the alpha chain is encoded by the HLA-C loci.

In embodiments, the HLA/ligand complex contains a beta-2 microglobulin domain (B2M), where the B2M domain is encoded by HLA gene complex.

In embodiments, the HLA/ligand complex contains a peptide ligand, e.g., a neoepitope cleaved from a neoantigen described herein. In embodiments, the peptide ligand (e.g., a neoepitope) is between 8 and 13 amino acid residues in length. In some embodiments, the peptide ligand (e.g., a neoepitope) is 8 amino acid residues in length. In some embodiments, the peptide ligand (e.g., a neoepitope) is 9 amino acid residues in length. In some embodiments, the peptide ligand (e.g., a neoepitope) is 10 amino acid residues in length. In some embodiments, the peptide ligand (e.g., a neoepitope) is 11 amino acid residues in length. In some embodiments, the peptide ligand (e.g., a neoepitope) is 12 amino acid residues in length. In some embodiments, the peptide ligand (e.g., a neoepitope) is 13 amino acid residues in length.

In embodiments, the HLA/ligand complex contains a peptide ligand, wherein the peptide ligand contains a non-natural amino acid (e.g., for peptide exchange assays). In some embodiments, the non-natural amino acid is activated by UV radiation. In some embodiments, the peptide ligand containing the non-natural amino acid is cleaved after irradiation by UV light. In some embodiments, the non-natural amino acid is selected from 2-nitrophenylglycine (NPG), expanded o-nitrobenzyl linker, o-nitrobenzylcaged phenol, o-nitrobenzyl caged thiol,32 nitroveratryloxycarbonyl (NVOC) caged aniline, o-nitrobenzyl caged selenides, bis-azobenzene, coumarin, cinnamyl, spiropyran, 2-nitrophenylalanine (2-nF), and 3-amino-3-(2-nitrophenyl)propionic acid (ANP) amino acid analogs. In some embodiments, the non-natural amino acid is 3-amino-3-(2-nitrophenyl)propionic acid (ANP).

In embodiments, the non-natural amino acid can be located at any position between the N- and C-termini of the peptide ligand. In some embodiments, the non-natural amino acid is located at the N-terminus of the peptide ligand. In some embodiments, the non-natural amino acid is located at the second position of the peptide ligand (i.e., second position from the N-terminus). In some embodiments, the non-natural amino acid is located at the third position of the peptide ligand. In some embodiments, the non-natural amino acid is located at the fourth position of the peptide ligand. In some embodiments, the non-natural amino acid is located at the fifth position of the peptide ligand. In some embodiments, the non-natural amino acid is located at the sixth position of the peptide ligand. In some embodiments, the non-natural amino acid is located at the seventh position of the peptide ligand. In some embodiments, the non-natural amino acid is located at the eighth position of the peptide ligand. In some embodiments, the non-natural amino acid is located at the ninth position of the peptide ligand. In some embodiments, the non-natural amino acid is located at the tenth position of the peptide ligand. In some embodiments, the non-natural amino acid is located at the C-terminus of the peptide ligand.

Non-limiting examples of HLA alleles may be found in the instant specification and figures. Additional alleles are known in the art.

Most mammals have MHC variants similar to those of humans, who bear great allelic diversity, especially among the nine classical genes-seemingly due largely to gene duplication-though human MHC regions have many pseudogenes (Sznarkowska et al., “MHC Class I Regulation: The Origin Perspective”. Cancers. 2020; 12(5): 1155). The most diverse loci, namely HLA-A, HLA-B, and HLA-C, have roughly 6000, 7200, and 5800 known alleles, respectively (see “HLA Alleles Numbers” at World Wide Web site at hla.alleles.org). Many HLA alleles are ancient, sometimes of closer homology to chimpanzee MHC alleles than to some other human alleles of the same gene.

The human leukocyte antigen (HLA) system or complex is a complex of genes on chromosome 6 in humans which encode cell-surface proteins responsible for the regulation of the immune system (Choo. “The HLA system: genetics, immunology, clinical testing, and clinical implications”. Yonsei Medical Journal. 2007; 48(1): 11-23). The HLA system is also known as the human version of the major histocompatibility complex (MHC) found in many animals.

In some embodiments, the MHC alleles (e.g., MHCI alleles) described herein include HLA alleles in human subjects. In some embodiments, the MHC alleles (e.g., MHCI alleles) described herein include non-human MHC alleles.

MHC alleles described herein may be carried by a subject having a tumor or cancer expressing a neoantigen, which may be cleaved into a plurality of neoepitopes. In some embodiments, a plurality of subjects (or tumors/cancers) may have a same neoantigen (the “shared neoantigen” scenario). In some embodiments, a subject (or tumor/cancer) may have a specific neoantigen or a plurality of specific neoantigens (e.g., at least one neoantigen different from the ones in other subjects) (the “personalized neoantigen” scenario). One aspect of the instant disclosure is about identification of neoepitopes being specifically recognized and presented by a specific MHC (e.g., MHCI or HLA) allele in a single or plurality of subjects. Such information is useful for designing cancer/tumor vaccines or T cell therapies in a subject expressing a specific MHC allele peptide.

In some embodiments, a plurality of subjects having a tumor or a cancer may have a same or different HLA alleles, which may be identified by e.g., genotyping these subjects. These subjects may have different neoantigen-associated peptides expressed by the tumor or cancer cells (e.g., different people may have different tumor/cancer-associated mutations). In some embodiments, a screen method (either targeted or untargeted) may be used to identify a subject among the plurality of subjects for immunotherapies, through identification of a specific neoepitope-HLA binding pair. For example, if a subject having a neoepitope cleaved from a neoantigen expressed by the tumor or cancer cells, capable of being recognized and presented by a specific HLA protein in the same subject by forming a neoepitope-HLA complex, then the subject is determined to be treatable by T cell therapies (e.g., through TCRs or CARs) utilizing T cells capable of recognizing and being activated by the neoepitope-HLA complex. In some embodiments, for a single or a plurality of subjects having a same HLA protein, if at least one neoepitope is identified for forming a neoepitope-HLA complex with the specific HLA protein, through the same method described herein, cancer/tumor vaccines may be prepared to contain the at least one neoepitope for administration to the subject(s) to induce immune response against the tumor or cancer.

In some aspects, the instant disclosure provides a general treatment strategy/plan/method for a plurality of subjects having a tumor or cancer. For example, the HLA alleles in each of the plurality of subjects may be determined (e.g., by genotyping the subjects). In some embodiments, the HLA allele information for each of the subjects were known from any of previous analyses. Then at least one specific neoepitope-HLA binding pair is identified by combining all (e.g., in an “untargeted” analysis) or some (e.g., in a “targeted” analysis) of the neoepitopes cleaved by all (e.g., in an “untargeted” analysis) or some (e.g., in a “targeted” analysis) of neoantigens in the subjects and specific HLA alleles in these subjects. Any known method may be used to test if a specific neoepitope-HLA binding pair is formed, such as a peptide exchange assay, a fluorescence assay, an immunological assay, etc. The subjects having a specific HLA allele which forms a neoepitope-HLA binding pair with a specific neoepitope may be determined as potentially treatable, and then be optionally treated, by cancer/tumor vaccines or T cell therapies targeting the neoepitope-HLA binding pair.

Methods of identifying specific neoepitope-HLA binding pairs are disclosed herein. In some embodiments, peptide exchange assays or other cell-based assays may be used. Other examples of assays may include, e.g., complex detection assays, HLA binding ligand identification assays, native SEC-MS and CE-MS methods, or others described in PCT Application No. PCT/EP2019/066811 (published as WO 2020/002320), the content of which is incorporated by reference herein to its entirety.

In some embodiments, monoallelic cells are used to test the binding between the HLA proteins with neoepitopes. For example, monoallelic cells may be prepared by knocking down or knocking out endogenous HLA alleles in cells and then transducing a single HLA allele into the cells for expression. A plurality of monoallelic cells may be prepared so that each cell expresses a different HLA allele, thus forming a group of monoallelic cells for identifying specific neoepitope-HLA binding pairs for a single or a plurality of neoepitopes.

### IV. Compositions

### Neoantigens and Neoepitopes

Nucleic acid compositions are disclosed for nucleic acids encoding neoantigen and/or neoepitope sequences. In some embodiments, at least one nucleic acid in the compositions described herein encodes a neoantigen and/or neoepitope having at least 70%, 75%, 80%, 85%, 90%, 91%, 92%, 93%, 94%, 95%, 96%, 97%, 98%, 99%, or 100% (and all sub-values and sub-ranges there between, including endpoints) sequence identity to neoantigen and/or neoepitope sequences described herein in the instant specification and figures. In some embodiments, at least one nucleic acid in the compositions described herein has at least 70%, 75%, 80%, 85%, 90%, 91%, 92%, 93%, 94%, 95%, 96%, 97%, 98%, 99%, or 100% (and all sub-values and sub-ranges there between, including endpoints) sequence identity to neoantigen and/or neoepitope nucleic acid sequences described herein in the instant specification and figures.

In some embodiments, a composition described herein comprises an isolated polynucleotide cassette encoding a plurality of neoantigens or neoepitopes. For example, the plurality of neoantigens may be expressed by tumor or cancer cells in a subject or a plurality of subjects. In other examples, the plurality of neoepitopes may be cleaved from a single neoantigen or a plurality of neoantigens expressed by tumor or cancer cells in a subject or a plurality of subjects. The isolated polynucleotide cassette may encode a polypeptide comprising a fusion of multiple neoantigens or neoepitopes, with or without linkers (e.g., peptide liners such as Glycine-Serine (GS) linkers) between two adjacent neoantigens or neoepitopes. In some embodiments, each of the neoantigens/neoepitopes encoded by the isolated polynucleotide cassette is between about 5 and about 100, about 5 and about 90, about 5 and about 80, about 5 and about 70, about 5 and about 60, about 5 and about 50, about 5 and about 40, about 5 and about 30, about 5 and about 20, about 10 and about 100, about 10 and about 90, about 10 and about 80, about 10 and about 70, about 10 and about 60, about 10 and about 50, about 10 and about 40, about 10 and about 30, about 10 and about 20, about 20 and about 90, about 20 and about 80, about 20 and about 70, about 20 and about 60, about 20 and about 50, about 20 and about 40, or about 20 and about 30 (and all sub-values and sub-ranges there between, including endpoints) amino acids in length. In some embodiments, each of the neoantigens/neoepitopes encoded by the isolated polynucleotide cassette is between about 20 and about 50 amino acids in length. In some embodiment, each of the neoantigens encoded by the isolated polynucleotide cassette can be cleaved in the cell to produce a plurality of neoepitopes, each of which may have about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acids in length. In some embodiments, each of the neoantigens encoded by the isolated polynucleotide cassette can be cleaved in the cell to produce a plurality of neoepitopes, each of which may have about 8, 9, 10, 11, 12 or 13 amino acids in length. In some embodiments, the isolated polynucleotide cassette encodes a plurality of neoepitopes, each of which may have about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acids in length. In some embodiments, the isolated polynucleotide cassette encodes a plurality of neoepitopes, each of which may have about 8, 9, 10, 11, 12 or 13 amino acids in length.

In some embodiments, an isolated polynucleotide cassette described herein may encode any number of neoantigens or neoepitopes described herein. For example, 24 or 47 neoantigens were prepared in a piggybac cassette containing a concatenated neoantigen expression array, with or without a peptide linker, in the experimentation illustrated in Examples and figures. There is generally no limitation on the exact number of neoantigens to be conjugated into a polynucleotide cassette, unless the cassette cannot be transduced to or expressed in cells. In some embodiments, the isolated polynucleotide cassette encodes at least 2 neoantigens, such as between 2 and 200 neoantigens, for example between 20 and 100, between 20 and 75, or between 30 and 50 neoantigens (and all sub-values and sub-ranges there between, including endpoints). In some embodiments, the isolated polynucleotide cassette encodes 2, 5, 10, 15, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80 or more neoantigens.

In some embodiments, a nucleic acid composition described herein (e.g., an isolated polynucleotide cassette) may be inserted into an expression vector (e.g., a plasmid or viral vector). In some embodiments, the expression vector may further comprise at least one promoter capable of initiating transgene expression and initiating translation of the plurality of neoantigens or neoepitopes into a single polypeptide in a cell. In some embodiments, selectable markers may be added to the expression vector, including expression markers and/or antibiotic resistance markers. In some embodiments, the expression vectors described herein are stably or transiently delivered into the cells, using methods described herein or known to a skilled artisan.

In some embodiments, a neoantigen described herein comprises a peptide expressed by a tumor or cancer cell in a subject. In some embodiments, the neoantigen-associated peptide comprises at least one mutation (including, e.g., a substitution, deletion, insertion, cross-linking, etc.) to at least one amino acid residue, compared to the sequence of the corresponding wild-type peptide expressed by a wild-type cell, a healthy cell, or a non-tumor or non-cancer cell. The difference between the sequence of the neoantigen-associated peptide and the wild-type peptide, e.g., caused by the at least one mutation, may render the neoantigen-associated peptide “not previously recognized” by the immune system in a subject (“not self”) and, thus, capable of inducing immune response in the subject. In some embodiments, a neoantigen described herein is between about 5 and about 100, about 5 and about 90, about 5 and about 80, about 5 and about 70, about 5 and about 60, about 5 and about 50, about 5 and about 40, about 5 and about 30, about 5 and about 20, about 10 and about 100, about 10 and about 90, about 10 and about 80, about 10 and about 70, about 10 and about 60, about 10 and about 50, about 10 and about 40, about 10 and about 30, about 10 and about 20, about 20 and about 90, about 20 and about 80, about 20 and about 70, about 20 and about 60, about 20 and about 50, about 20 and about 40, or about 20 and about 30 (and all sub-values and sub-ranges there between, including endpoints) amino acids in length. In some embodiments, a neoantigen described herein is between about 20 and about 50 amino acids in length.

In some embodiments, a neoantigen described herein comprises a neoantigen which is expressed by a tumor or cancer cell in a subject and may be further cleaved inside the tumor or cancer cell. Such cleavage may be induced by various proteinases or through the proteasome system. In some embodiments, a plurality of peptides may be produced by the cleavage of the neoantigen. For example, a plurality of peptides containing the at least one mutation differentiating the neoantigen from the corresponding wild-type peptide may be produced by the cleavage. In some embodiments, the plurality of peptides are then presented by the expressed HLA allele peptides on the cell surface for recognition by immune cells (e.g., T cells). Among the plurality of peptides, those with a potential capability to activate immune cells, upon recognition of the presented peptides, and thus induce immune response in the subject, may be determined as neoepitopes.

In some embodiments, a neoepitope described herein is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acids in length. In some embodiments, a neoepitope described herein is about 8, 9, 10, 11, 12 or 13 amino acids in length.

Examples of neoantigen and neoepitope sequences in the compositions described herein may be found in the instant specification and figures. In some embodiments, at least one of the neoantigen-associated peptides in the compositions described herein comprises at least 70%, 75%, 80%, 85%, 90%, 91%, 92%, 93%, 94%, 95%, 96%, 97%, 98%, 99%, or 100% (and all sub-values and sub-ranges there between, including endpoints) sequence identity to at least one of SEQ ID NOs: 1-72, 75-191, and 195-222. In some embodiments, at least one of the neoepitopes in the compositions described herein comprises at least 70%, 75%, 80%, 85%, 90%, 91%, 92%, 93%, 94%, 95%, 96%, 97%, 98%, 99%, or 100% (and all sub-values and sub-ranges there between, including endpoints) sequence identity to at least one of SEQ ID NOs: 75-111.

In some embodiments, the neoantigen and/or the neoepitopes described herein were known to a skilled artisan or identified by bioinformatics and/or a clinical analysis of tumor mutations.

### MHC Alleles

A MHC allele (e.g., a MHC class I, or MHCI, allele) described herein may include a MHC molecule (e.g., a MHCI or HLA molecule), which includes an alpha chain, a beta chain, and a ligand, wherein the ligand is a peptide comprising a non-natural UV-cleavable amino acid (e.g., for peptide exchange assays) or comprise a neoepitope described herein (e.g., for discovering or utilizing the neoepitope-HLA binding pair).

In embodiments, the HLA described herein contains an alpha chain. In some embodiments, the alpha chain is encoded by any one of the following loci: HLA-A, HLA-B, and HLA-C. In some embodiments, the alpha chain is encoded by the HLA-A loci. In some embodiments, the alpha chain is encoded by the HLA-B loci. In some embodiments, the alpha chain is encoded by the HLA-C loci. In embodiments, the HLA described herein further contains a beta chain. For example, the HLA described herein may contains an alpha chain and a beta chain in a single polypeptide (e.g., as a fusion protein). In embodiments, the HLA described herein does not contain a beta chain. For example, the HLA described herein (e.g., a nucleic acid encoding a HLA polypeptide) may be introduced into a cell expressing a beta chain. The beta chain described herein may comprise a B2-microglobulin (B2M). Unless expressly stated, expressing a HLA in a cell in the instant disclosure generally refers to expressing an alpha chain of the HLA in the cell.

In embodiments, the HLA may specifically bind to a peptide ligand, e.g., a neoepitope cleaved from a neoantigen described herein, and present the peptide ligand on the surface of the cell expressing the HLA. In embodiments, the peptide ligand (e.g., a neoepitope) is between 8 and 13 amino acid residues in length. In some embodiments, the peptide ligand (e.g., a neoepitope) is 8 amino acid residues in length. In some embodiments, the peptide ligand (e.g., a neoepitope) is 9 amino acid residues in length. In some embodiments, the peptide ligand (e.g., a neoepitope) is 10 amino acid residues in length. In some embodiments, the peptide ligand (e.g., a neoepitope) is 11 amino acid residues in length. In some embodiments, the peptide ligand (e.g., a neoepitope) is 12 amino acid residues in length. In some embodiments, the peptide ligand (e.g., a neoepitope) is 13 amino acid residues in length.

In embodiments, the peptide ligand contains a non-natural amino acid (e.g., for peptide exchange assays) as described herein. In some embodiments, the non-natural amino acid is activated by UV radiation. In some embodiments, the peptide ligand containing the non-natural amino acid is cleaved after irradiation by UV light. In some embodiments, the non-natural amino acid is selected from 2-nitrophenylglycine (NPG), expanded o-nitrobenzyl linker, o-nitrobenzylcaged phenol, o-nitrobenzyl caged thiol,32 nitroveratryloxycarbonyl (NVOC) caged aniline, o-nitrobenzyl caged selenides, bis-azobenzene, coumarin, cinnamyl, spiropyran, 2-nitrophenylalanine (2-nF), and 3-amino-3-(2-nitrophenyl)propionic acid (ANP) amino acid analogs. In some embodiments, the non-natural amino acid is 3-amino-3-(2-nitrophenyl)propionic acid (ANP).

Examples of HLA allele sequences may be found in the instant specification and figures.

Most mammals have MHC variants similar to those of humans, who bear great allelic diversity, especially among the nine classical genes-seemingly due largely to gene duplication-though human MHC regions have many pseudogenes (Sznarkowska et al., “MHC Class I Regulation: The Origin Perspective”. Cancers. 2020; 12(5): 1155). The most diverse loci, namely HLA-A, HLA-B, and HLA-C, have roughly 6000, 7200, and 5800 known alleles, respectively (see “HLA Alleles Numbers” at World Wide Web site at hla.alleles.org). Many HLA alleles are ancient, sometimes of closer homology to a chimpanzee MHC alleles than to some other human alleles of the same gene. Examples of HLA proteins, identified in neoepitope-MHC binding pairs, may include A*01.01, A*02.01, A*03.01, A*11.01, A*24.02, B*07.02, B*08.01, B*35.01, B*44.02, B*51.01, C*03.04, C*04.01, C*05.01, C*06.02, C*07.01, C*07.02, and C*08.02.

The human leukocyte antigen (HLA) system or complex is a complex of genes on chromosome 6 in humans which encode cell-surface proteins responsible for the regulation of the immune system (Choo. “The HLA system: genetics, immunology, clinical testing, and clinical implications”. Yonsei Medical Journal. 2007; 48(1): 11-23). The HLA system is also known as the human version of the major histocompatibility complex (MHC) found in many animals.

In some embodiments, the compositions comprising MHC molecules (e.g., MHCIs or HLAs) described herein include the compositions comprising HLA molecules in human subjects. In some embodiments, the compositions comprising MHC molecules (e.g., MHCIs) described herein include compositions comprising non-human MHC molecules.

Nucleic acid compositions are also disclosed for nucleic acids encoding MHC molecules. In some embodiments, at least one nucleic acid in the compositions described herein encodes a HLA allele peptide having at least 70%, 75%, 80%, 85%, 90%, 91%, 92%, 93%, 94%, 95%, 96%, 97%, 98%, 99%, or 100% (and all sub-values and sub-ranges there between, including endpoints) sequence identity to HLA allele peptide sequences described herein in the instant specification and figures. In some embodiments, at least one nucleic acid in the compositions described herein has at least 70%, 75%, 80%, 85%, 90%, 91%, 92%, 93%, 94%, 95%, 96%, 97%, 98%, 99%, or 100% (and all sub-values and sub-ranges there between, including endpoints) sequence identity to HLA allele nucleic acid sequences described herein in the instant specification and figures.

In some embodiments, a composition described herein comprises an isolated polynucleotide encoding a single or a plurality of HLA allele peptides. For example, the plurality of HLA allele peptides may be expressed in a subject or a plurality of subjects. The isolated polynucleotide may encode a polypeptide comprising a fusion of multiple HLA allele peptides. Examples of MHC proteins, such as HLAs, identified in neoepitope-MHC binding pairs, may include A*01.01, A*02.01, A*03.01, A*11.01, A*24.02, B*07.02, B*08.01, B*35.01, B*44.02, B*51.01, C*03.04, C*04.01, C*05.01, C*06.02, C*07.01, C*07.02, and C*08.02. Any one or more alleles may be expressly excluded, including HLA I alleles encoded by any one of the following loci: HLA-A, HLA-B, and HLA-C.

In some embodiments, a nucleic acid composition described herein (e.g., an isolated polynucleotide) may be inserted into an expression vector (e.g., a plasmid or viral vector). In some embodiments, the expression vector may further comprise at least one promoter capable of initiating translation of the HLA allele peptide(s) into a single polypeptide in a cell. In some embodiments, selectable markers may be added to the expression vector, including expression markers and/or antibiotic resistance markers. In some embodiments, the expression vectors described herein are stably or transiently delivered into the cells, using methods described herein or known to a skilled artisan.

As disclosed in more details in the following section of expression cells, a monoallelic cell may be prepared by knocking down or knocking out endogenous HLA alleles in the cell and further expressing an exogenous HLA protein introduced into the cell by an expression vector (e.g., a plasmid or viral vector). Such monoallelic cell, or a plurality of monoallelic cells, each of which expresses a different HLA protein, may be used to contact a plurality of neoepitopes to identify specific neoepitope-HLA binding pairs.

### Engineered Monoallelic Cells and Cell Lines

In an aspect, an engineered cell is provided to express at least one MHC allele, e.g. MHCI or HLA allele, described herein. In an aspect, an engineered cell is provided to express a single MHC allele, e.g. MHCI or HLA allele, described herein.

In some embodiments, the engineered cell is introduced with a MHC allele polynucleotide (e.g., the whole or part of a MHC allele gene or exons) for expression of a MHC allele peptide. In some embodiments, the MHC allele polynucleotide or peptide is a MHCI allele polynucleotide or peptide. In some embodiments, the MHCI allele peptide is encoded by any one of MHC allele genes of human or non-human animals, including, e.g., loci such as HLA-A, HLA-B, and HLA-C. In some embodiments, the HLA allele polynucleotide encodes an alpha chain. In some embodiments, the engineered cell expresses a beta chain for a HLA peptide, such as B2-microglobulin (B2M). In some embodiments, the HLA allele polynucleotide is introduced into the engineered cell as a HLA composition described herein. In some embodiments, the HLA allele polynucleotide is in an expression vector described herein. The HLA allele polynucleotide or peptide for introduction may be endogenous or exogenous to the cell. In embodiments, the HLA allele polynucleotide or peptide for introduction is exogenous to the cell.

In some embodiments, the engineered cell is a monoallelic cell, that is, expresses only one MCHI allele. Without limitation, the cell may be engineered to reduce or completely inhibit expression of all endogenous HLA alleles except for one, resulting in a cell expressing only one type of HLA allele peptides. In other examples, the cell may be engineered to reduce or completely inhibit expression of all endogenous HLA alleles and then be introduced with a HLA allele polynucleotide for expression or a HLA allele peptide, resulting in a cell expressing only one type of HLA allele peptides. Examples of reducing or completely inhibiting expression of endogenous HLA allele expression may include, at least, knockout methods (e.g., CRISPR technology) and knockdown methods (e.g., siRNAs, shRNAs, antisense oligonucleotides, miRNAs, etc.). In embodiments, the HLA allele is transiently expressed. In embodiments, the HLA allele is stably expressed.

In some embodiments, the engineered cell is further introduced with a polypeptide, or a polynucleotide encoding such polypeptide, comprising a single or a plurality of neoantigens or neoepitopes. When introduced into the engineered cell, the single or plurality of neoantigens may be cleaved to produce a plurality of neoepitopes. In some embodiments, the neoepitopes bind to HLA allele peptides expressed by the engineered cell and are presented on the cell surface. In some embodiments, at least one of the neoepitopes binds to the single type of HLA expressed by the monoallelic engineered cell and is presented on the cell surface. In some embodiments, the polypeptide, or a polynucleotide encoding such polypeptide, comprises a single polypeptide or polynucleotide chain comprising a fusion of a plurality of the neoantigens or neoepitopes, with or without linker(s) described herein between two adjacent neoantigens or neoepitopes, or their coding polynucleotides.

In some embodiments, the engineered cell is further introduced with a polynucleotide cassette encoding a plurality of neoantigen-associated peptides or neoepitopes, as described herein. Examples of polynucleotide cassettes may include the piggybac expression constructs, described in the section of Examples, comprising 24 or 47 neoantigens. Generally there is no specific limitation on the exact number of neoantigens or neoepitopes for fusion into the polynucleotide cassette, unless the transduction to or the expression of the cassette in the cell is adversely affected or inhibited. In some embodiments, the isolated polynucleotide cassette encodes at least 2 neoantigens, such as between 2 and 200 neoantigens, for example between 20 and 100, between 20 and 75, or between 30 and 50 neoantigens (and all sub-values and sub-ranges there between, including endpoints). In some embodiments, the isolated polynucleotide cassette encodes 2, 5, 10, 15, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80 or more neoantigens.

Generally, there is no specific limitation on cell types for engineering described herein. In some embodiments, the engineered cell is any type of nucleated cells. In some embodiments, the engineered cell is an animal cell. In some embodiments, the engineered cell is a mammalian cell, such as a human cell or a non-human mammalian cell. In some embodiments, the engineered cell is an antigen-presenting cell (APC) or is capable of presenting antigens (e.g., the neoepitopes expressed in the cell and/or cleaved from the expressed neoantigen-associated peptides in the cell) on the cell surface for recognition by an immune cell (e.g., a T cell). In some embodiments, the engineered cell is a HMy2.C1R cell. In some embodiments, the engineered cell is a K562 cell (e.g., ATCC product CCL-243™).

In an aspect, a cell line is provided, which includes a plurality of monoallelic cells described herein. In embodiments, the cells of the cell line stably or transiently express a single HLA allele.

In an aspect, a plurality of cell lines is provided. In embodiments, each cell line includes cells that stably or transiently express a single HLA allele. In embodiments, cell lines expresses different HLA alleles. In embodiments, each cell line expresses a different HLA allele. In embodiments, each cell line expresses at least one different HLA allele.

### Tumor Cancer Vaccines

In an aspect, a tumor vaccine or a cancer vaccine is provided for inducing an immune response in a subject or a plurality of subjects having a tumor or cancer, or being prone to the tumor or cancer. Examples of the tumor/cancer vaccines include, at least, treatment vaccines or therapeutic vaccines capable of keeping the cancer from coming back, induce targeting/destruction of any cancer cells still in the body after treatments end, stopping a tumor from growing or spreading, etc.

In some embodiments, the tumor/cancer vaccine described herein comprises a polypeptide, or a polynucleotide encoding such polypeptide, comprising a neoantigen or a neoepitope cleaved from the neoantigen expressed by the tumor or cancer cell. In some embodiments, the tumor/cancer vaccine described herein comprises a neoantigen or neoepitope composition described herein.

In some embodiments, at least one of the neoepitopes or the neoepitopes cleaved from the neoantigen(s) in the tumor/cancer vaccine described herein forms a specific neoepitope-HLA binding pair with a HLA expressed by the tumor or cancer cell, or another APC cell, in the subject(s) to be administered with the vaccine. In non-limiting examples, a fraction or all of HLA alleles expressed in the subject(s) are known prior to administration with the vaccine. Examples of identifying HLA allele sub-types in the subject(s) may include, at least, genotyping or other sequencing methods known to a skilled artisan. Provided with the information of the HLA allele sub-types in the subject(s), a method described herein may be used to identify neoepitope-HLA binding pairs comprising neoepitopes specifically recognized by the HLA allele sub-types in the subject(s). Such neoepitopes, or the corresponding neoantigens from which the neoepitopes are cleaved, may be used to prepare a tumor/cancer vaccine described herein for administration to the subject(s).

In some embodiments, a single or a plurality of subjects may be sequenced or genotyped to provide information about the specific HLA allele sub-types in the subject(s) for identifying neoepitope-HLA binding pairs for the specific HLA allele sub-types, while the identified neoepitopes, or the corresponding neoantigen(s) from which the neoepitopes are cleaved, may be used to prepare a tumor/cancer vaccine described herein for administration to the subject(s).

In some embodiments, the tumor/cancer vaccine comprising a neoantigen or neoepitope described herein are known to a skilled artisan. In non-limiting examples, a single or a plurality of subjects may be sequenced or genotyped to provide information about the specific HLA allele sub-types in the subject(s) for identifying neoepitope-HLA binding pairs for the specific HLA allele sub-types by a method described herein. Any subject having the specific HLA allele sub-types in the identified binding pairs with a specific neoepitope comprised in, or cleavable by a neoantigen comprised in, the tumor/cancer vaccine may be determined as a subject for administration of the vaccine for treating or preventing the tumor/cancer. In non-limiting examples, a screening method described herein may be applied to the single or plurality of subjects to identify a subgroup of subjects to be administered with the vaccine.

### Engineered T Cells

In an aspect, an engineered T cell is provided. In embodiments, the engineered T cell includes a nucleic acid sequence encoding a polypeptide comprising an exogenous TCR-beta and an exogenous TCR-alpha (VJ) domain. In embodiments, the nucleic acid sequence is inserted into a TCR-alpha locus of the engineered T cell. In embodiments, the engineered T cell includes a nucleic acid sequence encoding a chimeric antigen receptor (CAR) polypeptide.

In another interrelated aspect, a composition comprising isolated T cells is provided, wherein at least 5% of the cells are engineered T cells, each engineered T cell including a nucleic acid sequence encoding a polypeptide comprising an exogenous TCR-beta and an exogenous TCR-alpha (VJ) domain. In embodiments, the nucleic acid sequence is inserted into a TCR-alpha locus of the engineered T cell.

In embodiments, expression of an endogenous TCR-beta gene was disrupted by gene editing.

In embodiments, the exogenous TCR-alpha (VJ) domain forms part of a heterologous TCR-alpha comprising at least a portion of the endogenous TCR-alpha of the T cell. In embodiments, the TCR-alpha locus is a TCR-alpha constant region. In embodiments, the exogenous TCR-beta and the heterologous TCR-alpha are expressed from the nucleic acid and form a functional TCR. In embodiments, the engineered T cell is bound to an antigen. In embodiments, the engineered T cell is bound to a cancer cell. In embodiments, the TCR binds to the antigen presented on a major histocompatibility complex class I (MHCI) molecule.

In embodiments, the antigen is a neoantigen (e.g., a tumor-associated antigen (TAA)) or a neoepitope cleaved or cleavable from the neoantigen. In embodiments, the antigen is a neoantigen. In embodiments, the antigen is a neoepitope. In embodiments, the neoantigen or TAA is selected from WT1, JAK2, NY-ISO1, PRAME, KRAS, or an antigen from Table 1 or Table 2. In embodiments, the antigen is specific to a cancer of a subject to be administered the engineered T cell. In embodiments, the antigen is expressed by or associated with a cancer of a subject to be administered the engineered T cell.

In embodiments, the nucleic acid sequence further encodes a self-cleaving peptide. In embodiments, the self-cleaving peptide is a self-cleaving viral peptide. In embodiments, the self-cleaving viral peptide is T2A. In embodiments, the self-cleaving viral peptide is P2A. In embodiments, the self-cleaving viral peptide is E2A. In embodiments, the self-cleaving viral peptide is F2A.

In embodiments, the engineered T cell expresses CD45RO, C-C chemokine receptor type 7 (CCR7), and L-selectin (CD62L). In embodiments, the engineered T cell has a central memory (CM) T cell phenotype. In embodiments, the engineered T cell has a naïve T cell phenotype. In embodiments, the engineered T cell having a naïve T cell phenotype is CD45RA+CD45RO−CD27+CD95−(that is, the cell expresses CD45RA and CD27 and does not express detectable levels of CD45RO and CD95). In embodiments, the engineered T cell has a stem cell memory T cell phenotype. In embodiments, the engineered T cell having a stem cell memory T cell phenotype is CD45RA+CD45RO-CD27+CD95+CD58+CCR7-Hi TCF1+. In embodiments, the engineered T cell has a central memory T cell phenotype. In embodiments, the engineered T cell having a central memory T cell phenotype is CD45RO+CD45RA−CD27+CD95+CD58+. In embodiments, the engineered T cell has a progenitor exhausted T cell phenotype. In embodiments, the engineered T cell having a progenitor exhausted T cell phenotype is PD-1+SLAMF6+TCF1+TIM3−CD39 −. In embodiments, the engineered T cell having a progenitor exhausted T cell phenotype expresses PD-1 at a low or intermediate level. Expression levels of each marker can be compared to a control, such as (without limitation) a cell type known to express the marker, a cell type known not to express the marker, a cell population, a population of T cells, etc.

In embodiments, the T cell is autologous to a subject in need thereof. In embodiments, the T cell is allogeneic to a subject in need thereof.

In another interrelated aspect, a pharmaceutical composition is provided. The pharmaceutical composition includes a plurality of the engineered Ti9 cells as described herein, including embodiments, and a pharmaceutically acceptable excipient.

In embodiments, at least 10% of the cells in the composition comprising isolated T cells are engineered T cells. In embodiments, at least 20% of the cells are engineered T cells. In embodiments, at least 30% of the cells are engineered T cells. In embodiments, at least 40% of the cells are engineered T cells. In embodiments, at least 50% of the cells are engineered T cells. In embodiments, at least 60% of the cells are engineered T cells. In embodiments, at least 70% of the cells are engineered T cells. In embodiments, at least 80% of the cells are engineered T cells. In embodiments, at least 90% of the cells are engineered T cells.

In embodiments, the composition includes between about 0.1×10⁵ and about 1×10⁹ engineered T cells. In embodiments, the composition includes at least 1×10⁸ engineered T cells. In embodiments, the composition includes at least 1×10⁹ engineered T cells. The number of cells may be any value or subrange between the recited ranges, including endpoints.

In embodiments, the composition further includes a pharmaceutically acceptable excipient. The means of making such a composition or an implant have been described in the art (see, for instance, Remington's Pharmaceutical Sciences, 16th Ed., Mack, ed. (1980)). Where appropriate, the engineered T cells can be formulated into a preparation in semisolid or liquid form, such as a capsule, solution, injection, inhalant, or aerosol, in the usual ways for their respective route of administration. In embodiments, the excipient is a balanced salt solution, such as Hanks' balanced salt solution, or normal saline. It will also be understood that, if desired, the compositions of the invention may be administered in combination with other agents as well, such as, e.g., cytokines, growth factors, hormones, small molecules, chemotherapeutics, pro-drugs, drugs, antibodies, or other various pharmaceutically-active agents. There is virtually no limit to other components that may also be included in the compositions, provided that the additional agents do not adversely affect the ability of the composition to deliver the intended therapy.

In non-limiting examples, the engineered T cell described herein expresses a TCR (endogenous or exogenous) or a CAR molecule which specifically recognizes a neoepitope-HLA binding pair on the engineered cells (e.g., monoallelic cells) described in the above section. In some embodiments, the neoepitope-HLA binding pair presents the neoepitope on the cell surface, which is recognized by and bound to the expressed TCR or CAR molecule through the extracellular antigen-binding domain of the TCR or CAR molecule. Such recognition may lead to activation and proliferation of the engineered T cells, which may further increase their capacity to produce anti-tumor/cancer cytokines and/or cytotoxicity towards the tumor/cancer cells.

In non-limiting examples, a single or plurality of subjects may be sequenced or genotyped to provide information about the specific HLA allele sub-types in the subject(s) for identifying neoepitope-HLA binding pairs for the specific HLA allele sub-types by a method described herein. Engineered T cells described herein may be prepared to specifically recognize the neoepitope-HLA binding pairs, thus used as a therapy for the subject(s) having at least one identified neoepitope-HLA binding pair.

In some embodiments, a screening method described herein may be applied to a single or plurality of subjects to identify a subgroup of subjects to be administered with engineered T cells, which specifically recognize the neoepitope-HLA binding pair(s) in the subject(s), identified using the HLA allele sub-type information of the subject(s).

### V. Systems

In an aspect, provided herein, is a system containing a plurality of monoallelic HLA-expressing cell lines described herein. In some embodiments, the cells in each cell line do not express an endogenous HLA allele. In some embodiments, the cells in each cell line express an exogenous HLA allele described herein. In some embodiments, each cell line expresses a different exogenous HLA allele described herein. In some embodiments, the cells in each cell line express a beta chain of HLA complex, such as 2-microglobulin (B2M). In some embodiments, each cell line comprises a polynucleotide cassette encoding a plurality of neoantigen-associated peptides as described herein.

In some embodiments, the system described herein comprises a parent cell to be engineered to produce a monoallelic cell line using a method described herein. In some embodiments, the system described herein further comprises a polynucleotide cassette encoding a plurality of neoantigen-associated peptides as described herein.

In some embodiments, the system described herein comprises arrays or libraries of MHC (e.g., MHCI) alleles and/or a library of known or putative neoantigens or neoepitopes, for example, based on a pathogenic disease, type of tumor, or the like, and/or cells expressing the HLAs and/or neoantigens or neoepitopes. Such HLA allele and neoantigen or neoepitopes may be used to identify specific neoepitope-HLA binding pairs described herein.

### VI. Kits

In an aspect, provided herein, is a kit or reagent composition containing a system or a composition described herein. Kits or reagent compositions herein may also include reagents for preparing an engineered cell line, performing assays for identifying specific neoepitope-HLA binding pairs, and/or analyzing the binding between neoepitopes and HLA proteins, as described herein. Kits or reagent compositions herein may also include instructions for performing methods herein or portions of such methods.

### VII. Methods of Making

In an aspect, provided herein, is a method of producing the compositions described herein.

In one aspect, the instant application provides a method of producing the neoantigen and/or neoepitope compositions or the HLA compositions described herein. A single or plurality of polynucleotides encoding a neoantigen or neoepitope or a HLA peptide may be engineered (e.g., isolated, cloned, etc.) using a suitable method or a method known to a skilled artisan. Expression vectors (e.g., plasmids, viral vectors, etc.) may be used to introduce the neoantigen, the neoepitope, and/or the HLA composition into cells. Selectable markers may be used to identify engineered cells containing such expression vectors or polynucleotides for expression. In some embodiments, the expression vectors described herein are stably or transiently delivered into the cells, using methods described herein or known to a skilled artisan.

In another, interrelated aspect, a method for making an engineered (e.g., monoallelic) cell and/or cell line is provided. In some embodiments, the engineered cell is introduced with a HLA allele polynucleotide (e.g., the whole or part of a HLA allele gene or exons) for expression of a HLA allele peptide, through a suitable method or a method known to a skilled artisan. In some embodiments, the cell is engineered to reduce or completely inhibit expression of all endogenous MHC alleles (e.g., MHCI alleles) except for one, resulting in a cell expressing only one type of HLA allele peptides. In some embodiments, the cell is engineered to reduce or completely inhibit expression of all endogenous MHC alleles (e.g., MHCI alleles) and then be introduced with a MHC allele polynucleotide for expression of a MHC allele peptide, resulting in a cell expressing only one type of MHC allele peptides. Examples of reducing or completely inhibiting expression of endogenous HLA allele expression may include, at least, knockout methods (e.g., CRISPR technology) and knockdown methods (e.g., siRNAs, shRNAs, antisense oligonucleotides, miRNAs, etc.).

In some embodiments, the engineered cell is further introduced with a polypeptide, or a polynucleotide encoding such polypeptide, comprising a single or a plurality of neoantigens or neoepitopes. When introduced into the engineered cell, the single or plurality of neoantigens may be cleaved to produce a plurality of neoepitopes. In some embodiments, the engineered cell is further introduced with a polynucleotide cassette encoding a plurality of neoantigen-associated peptides or neoepitopes, as described herein.

In another, interrelated aspect, a method for making a tumor/cancer vaccine is provided. In some embodiments, the tumor/cancer vaccine described herein comprises a polypeptide, or a polynucleotide encoding such polypeptide, comprising a neoantigen or a neoepitope cleavable from the neoantigen expressed by the tumor or cancer cell. In some embodiments, the tumor/cancer vaccine described herein comprises a neoantigen or neoepitope composition described herein. In some embodiments, at least one of the neoepitopes or the neoepitopes cleavable from the neoantigen(s) in the tumor/cancer vaccine described herein forms a specific neoepitope-HLA binding pair with a HLA expressed by the tumor or cancer cell, or another APC cell, in a single or plurality of subject(s) to be administered with the vaccine.

In non-limiting examples, neoantigens may be expressed by the tumor or cancer cell in a single or plurality of subject(s). Based on the information of specific HLA allele information of the subject(s), specific neoepitope-HLA binding pairs are identified using a method described herein. In some embodiments, a tumor/cancer vaccine described herein is produced to comprise a neoepitope, or a neoantigen from which the neoepitope is cleaved, in the identified neoepitope-HLA binding pairs. In some embodiments, at least one tumor/cancer vaccine is selected from a plurality of vaccines for having the neoepitope, or the neoantigen from which the neoepitope is cleaved, in the identified neoepitope -HLA binding pairs. In some embodiments, at least one subject is selected from a plurality of subjects to be administered with a tumor/cancer vaccine, while the HLA in the subject and the neoepitope, or the neoantigen from which the neoepitope is cleaved, in the vaccine are capable of forming an identified neoepitope HLA binding pair.

In another, interrelated aspect, a method for making an engineered T cell is provided. In embodiments, the engineered T cell includes a nucleic acid sequence encoding a polypeptide comprising an exogenous TCR-beta and an exogenous TCR-alpha (VJ) domain. In embodiments, the nucleic acid sequence is inserted into a TCR-alpha locus of the engineered T cell. In embodiments, the engineered T cell includes a nucleic acid sequence encoding a chimeric antigen receptor (CAR) polypeptide.

In non-limiting examples, the engineered T cell described herein expresses a TCR (endogenous or exogenous) or a CAR molecule which specifically recognizes a neoepitope-HLA binding pair on the engineered cells (e.g., monoallelic cells) described in the above section. In non-limiting examples, a single or plurality of subjects may be sequenced or genotyped to provide information about the specific HLA allele sub-types in the subject(s) for identifying neoepitope-HLA binding pairs for the specific HLA allele sub-types by a method described herein. Engineered T cells described herein may be prepared by introducing T cells with a TCR or CAR molecule specifically recognizing the neoepitope-HLA binding pairs. In some embodiments, a screening method described herein may be applied to a single or plurality of subjects to identify a subgroup of subjects to be administered with engineered T cells, which specifically recognize the neoepitope-HLA binding pair(s) in the subject(s), identified using the HLA allele sub-type information of the subject(s).

### VIII. Methods of Use

### Methods of Identifying a Neoepitope-HLA Binding Pair

In an aspect, provided herein, is a method of identifying a neoepitope-HLA binding pair.

In some embodiments, the first optional step of the method includes providing or collecting engineered cells, such as a monoallelic HLA-expressing cell line comprising cells, wherein each cell expresses a first exogenous HLA and comprises a polynucleotide, e.g., a polynucleotide cassette, encoding a plurality of neoantigen-associated peptides or neoepitopes. The second optional step of the method includes expressing the plurality of neoantigens or neoepitopes in each cell, wherein a plurality of neoepitopes is produced by cleaving the plurality of neoantigen-associated peptides within each cell, such that one or more neoepitopes bind to the first exogenous HLA allele at the cell surface. The third optional step of the method includes eluting the neoepitope bound to the first exogenous HLA at the cell surface from the HLA using a suitable eluting solution. The fourth optional step of the method includes identifying the eluted neoepitope(s) from the third optional step, thereby identifying a neoepitope-HLA binding pair. Such plurality of neoantigen-associated peptides may be determined to bind to one or more HLAs by peptide exchange assay, or identified by bioinformatics and/or a clinical analysis of tumor mutations.

In some embodiments, the first optional step of the method includes providing or collecting engineered cells, such as a monoallelic HLA-expressing cell line comprising cells, wherein each cell expresses a first exogenous HLA allele. The second optional step of the method includes contacting the cells with a synthesized neoepitope. The third optional step of the method includes eluting peptides bound to the first exogenous HLA allele at the cell surface from the HLA allele using a suitable eluting solution. The fourth optional step of the method includes identifying the eluted neoepitope(s) from the third optional step, thereby identifying a neoepitope-HLA binding pair. In some embodiments, a plurality of synthesized neoepitopes is used in the second optional step, providing a screening for neoepitopes capable of forming binding pairs with the specific HLA protein.

In embodiments, the monoallelic cell line is a monoallelic cell line as described (or made as described) herein. In embodiments, the polynucleotide cassette is a polynucleotide cassette as described herein.

In embodiments, the synthesized neoepitope contains a detectable moiety. In embodiments, the detectable moiety is a heavy amino acid. The term “heavy amino acid” refers to the presence of one or more heavy isotopes, such as non-radioactive isotopes. Suitable isotopes include, for example, ²H, ¹³C, ¹⁵N or ¹⁸O. In embodiments, identification of an eluted neoepitope includes detection of the detectable moiety.

In embodiments, the synthesized neoepitope is determined to bind to one or more HLAs by peptide exchange assay.

In embodiments, the steps are repeated in a second monoallelic HLA-expressing cell line comprising cells, wherein each cell expresses a second exogenous HLA allele polypeptide. In embodiments, the steps are repeated in multiple cell lines, wherein each cell line expresses a different exogenous HLA allele polypeptide.

Methods of selecting subjects for treatment

In an aspect, provided herein, is a method of selecting a subject having a cancer or tumor for immune therapies. In some embodiments, the method is for selecting a subject for a T cell therapies, such as using a T cell expressing a T cell receptor (TCR) and/or a chimeric antigen receptor (CAR).

In some embodiments, the first optional step of the method includes genotyping a single or plurality of subjects to identify HLA allele(s) expressed by the subject(s) and neoantigens, or neoepitopes cleavable from the neoantigens, expressed by a cancer or tumor either in the subject(s) or prone to develop in the subject(s). In some embodiments, such HLA allele and the neoantigen/neoepitope information of the subject(s) is known to a skilled artisan. The second optional step of the method includes determining whether any of the expressed HLA protein(s) in the subject(s) is capable of forming specific binding pairs with one or more of the neoepitopes in the same subject. In non-limiting example, if at least a neoepitope-HLA binding pair is identified, the subject having both the neoepitope, or the neoantigen from which the neoepitope is cleaved, and the HLA allele may be determined as being treatable by the T cell comprising the TCR or CA specifically binds to the neoepitope-HLA binding pair, and, optionally, be treated by the T cell.

In some embodiments, the method is for selecting a subject for a cancer/tumor vaccine therapy.

In some embodiments, the first optional step of the method includes genotyping a single or plurality of subjects to identify HLA allele(s) expressed by the subject(s) and neoantigens, or neoepitopes cleavable from the neoantigens, expressed by a cancer or tumor either in the subject(s) or prone to develop in the subject(s). In some embodiments, such HLA allele and the neoantigen/neoepitope information of the subject(s) is known to a skilled artisan. The second optional step of the method includes determining whether any of the expressed HLA allele(s) in the subject(s) is capable of forming specific binding pairs with one or more of the neoepitopes. In non-limiting example, if at least a neoepitope-HLA binding pair is identified, the subject having the HLA may be determined as being treatable by a tumor/cancer vaccine comprising the neoepitope, or the neoantigen from which the neoepitope is cleaved, in the identified neoepitope-HLA binding pair having the HLA allele in the subject, and, optionally, be treated by the vaccine.

In embodiments, whether a neoepitope and a HLA form a neoepitope-HLA binding pair (e.g., identification of a neoepitope-HLA binding pair) is determined by analysis of a database of such interactions. In embodiments, the binding pairs in the database were determined, at least in part, using methods described therein.

Methods of treating a subject

In an aspect, provided herein, is a method of treating a subject having a cancer or tumor or preventing a subject from having/developing a cancer or tumor.

In some embodiments, the method includes administering to a subject a therapeutically effective amount of T cells expressing a T cell receptor (TCR) and/or a chimeric antigen receptor (CAR). In some embodiments, the TCR and/or the CAR specifically binds to a neoepitope-HLA binding pair comprising the HLA expressed in the subject and the neoepitope expressed by a tumor or cancer cell in the subject or prone to develop in the subject.

In some embodiments, the method includes selecting subjects for treatment as described in the above section and treating the selected subjects with compositions described herein, including a tumor/cancer vaccine or a T cell therapy with engineered T cells.

In some embodiments, the first optional step of the method includes selecting a subject expressing a HLA allele and having a cancer expressing a neoantigen. Optionally, the HLA is known or determined to bind to a neoepitope cleavable from the neoantigen. In some embodiments, the second optional step of the method includes administering to the subject a therapeutically effective amount of T cells expressing a T cell receptor (TCR) and/or a chimeric antigen receptor (CAR) described herein. Such TCR and/or the CAR may specifically bind to a neoepitope-HLA binding pair comprising the HLA and the neoepitope.

In some embodiments, the method includes administering to a subject a therapeutically effective amount of a vaccine comprising the neoepitope or a polynucleotide encoding the neoepitope. Such neoepitope may specifically bind to the HLA expressed in the subject to form a specific neoepitope-HLA binding pair.

Dosages for formulations and administration routes useful for treatment described herein may be determined as suitable by a medical doctor. For engineered T cells, an expanded plurality of engineered T cells may be administered to a subject in any suitable amount, such as between about 1×10⁵ and 1×10⁹ cells. In embodiments, the expanded plurality of engineered T cells comprises at least 1×10⁸ engineered T cells. In embodiments, the expanded plurality of engineered T cells comprises at least 1×10⁹ engineered T cells. The number may be any value or subrange within the recited ranges, including endpoints.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

One skilled in the art would understand that descriptions of making and using the complexes described herein is for the sole purpose of illustration, and that the present disclosure is not limited by this illustration.

The publications discussed herein are provided solely for their disclosure prior to the filing date of the present application. Nothing herein is to be construed as an admission that such publications constitute prior art to the claims appended hereto. All publications referenced herein are incorporated herein by reference in their entireties for all of their teachings, including, but not limited to, all compositions, components, reagents, and methods.

### EXAMPLES

Additional embodiments are disclosed in further detail in the following examples, which are provided by way of illustration and are not in any way intended to limit the scope of this disclosure or the claims.

### EXAMPLE 1: Neoepitope Discovery Across Shared Neoantigens: Biochemical, Cell Engineering and Mass Spec Analysis

Neoantigen-specific T cells play a critical role in immune-mediated elimination of tumors and significant resources have been dedicated to developing clinically active drugs that amplify the cancer immunity cycle to improve the magnitude and breadth of elicited immune responses (Chen and Mellman, Oncology meets immunology: the cancer-immunity cycle. Immunity 2013; 39(1): 1-10; Rosenberg, Decade in review-cancer immunotherapy: entering the mainstream of cancer treatment. Nat Rev Clin Oncol. 2014; 11(11):630-2). Whether alone or paired with broad immune system activation, targeted immunotherapeutics may enable enhanced potency and safety profiles (Melero et al., Evolving synergistic combinations of targeted immunotherapies to combat cancer. Nat Rev Cancer. 2015; 15(8):457-72; Panchal et al., Role of targeted immunotherapy for pancreatic ductal adenocarcinoma (PDAC) treatment: An overview. Int Immunopharmacol. 2021; 95:107508). Further, T cells programmed to eradicate neoantigen-expressing cells may facilitate design of next-generation cell therapies, particularly against solid tumors (Leidner et al., Neoantigen T-Cell Receptor Gene Therapy in Pancreatic Cancer. N Engl J Med. 2022; 386(22):2112-2119; Yang and Rosenberg, Adoptive T-Cell Therapy for Cancer. Adv Immunol. 2016; 130:279-94).

Over the past 10 years, Major Histocompatibility Complex Class I (MHCI) presentation of epitopes derived from cancer specific mutations (e.g., neoepitopes) has emerged as the critical mode of action by which our immune system can control tumor growth. Presentation of these non-self neoepitopes have been shown to induce a neoantigen-specific T cell response via the cancer immunity cycle that can drive an anti-tumor immune response. Because of the role neoantigen-specific T cells play in killing tumors, significant resources across academia and biotechnology have been dedicated to developing clinically active drugs that will amplify the cancer immunity cycle and improve the magnitude and breadth of the neoantigen specific T cell responses, including checkpoint inhibitors, cytokines and TNF superfamily agonists. For these therapeutic modalities, the primary goal is to amplify the entire immune response rather than a targeted therapy that selectively enhances specific neoantigen T cell responses so these treatments are agonistic to the actual neoantigens and neoepitopes presented on a given tumor.

There have been studies to amplify specific neoantigen T cell responses through the use of vaccines and engineered T cell therapies. These types of therapies target two broad categories of neoantigens, shared neoantigens or personalized/private neoantigens (Zhang et al., Neoantigen: A New Breakthrough in Tumor Immunotherapy. Front Immunol. 2021; 12:672356). Private (a.k.a., personalized) neoantigens represent the vast majority of mutations that arise during cancer progression and are somatic mutations unique to an individual's tumor and are not found across multiple patients or indications (Jhunjhunwala et al., Antigen presentation in cancer: insights into tumour immunogenicity and immune evasion. Nat Rev Cancer. 2021; 21(5):298-312). Developing therapeutics against private neoantigens necessitates development of a personalized drug, which poses several unique challenges and requires a complex process of genomic analysis of a patient's biopsy, HLA typing, and bioinformatics-based neoantigen prediction to rank epitopes all prior to designing and producing the therapeutic (Capietto et al., Characterizing neoantigens for personalized cancer immunotherapy. Curr Opin Immunol. 2017; 46:58-65; Lang et al., Identification of neoantigens for individualized therapeutic cancer vaccines. Nat Rev Drug Discov. 2022; 21(4):261-282). Although some success has been evident in this area (Ott et al., An immunogenic personal neoantigen vaccine for patients with melanoma. Nature 2017; 547(7662):217-221; Sahin et al., Personalized RNA mutanome vaccines mobilize poly-specific therapeutic immunity against cancer. Nature 2017; 547(7662): 222-226), the road to broadly available personalized neoantigen-targeting therapies has yet to be established.

In contrast to the individualized nature of personalized neoantigens, there is a growing number of mutations that are being identified across a wide scope of patients and indications, referred to as shared neoantigens (Zhang et al., Front Immunol. 2021). The prevalence of shared neoantigens is related to their biological function and many putative shared neoantigens derive from oncogenic mutations within proteins such as KRAS, EGFR, TP53 and BRAF (Klebanoff and Wolchok, Shared cancer neoantigens: Making private matters public. J Exp Med. 2018; 215(1):5-7). Prior knowledge of the specific mutation enables discovery and validation of target epitopes as well as a path towards “off the shelf” therapeutics that can be administered to any patient whose tumor bears the target mutation and appropriate HLA haplotype. Early examples of vaccines targeting shared neoantigens have shown promising preclinical efficacy, including vaccines targeting IDH1 (Schumacher et al., Nature 2014; 512(7514): 324-327), KRAS (Wang et al., Cancer Immunol Res. 2016; 4(3): 204-214) and H3.3K27M (Chheda et al., J Exp Med. 2018; 215(1): 141-157). In addition to vaccines, T cell therapies targeting shared neoantigens have recently shown clinical efficacy. Some of the early evidence showed that T cells specific to the KRAS G12D HLA-C*08:02 restricted neoepitopes were capable of producing an effective anti-tumor response in a human patient with lung metastatic tumors (Tran et al., N Engl J Med. 2016; 375(23):2255-2262; Leidner et al., Neoantigen T-Cell Receptor Gene Therapy in Pancreatic Cancer. N Engl J Med. 2022; 386(22):2112-2119). Efficacy has also been demonstrated in preclinical models for KRAS G12V/G12D HLA-A*11:01 restricted neoepitopes (Wang et al., Cancer Immunol Res. 2016; 4(3): 204-214). However, despite the potential of “off the shelf” vaccines and T cell therapies, identifying shared neoantigen epitopes and the HLA contexts in which they are presented remains a primary challenge to drug development.

Neoantigen specific T cell responses to tumor associated mutations require processing of the neoantigen into neoepitopes, peptides derived from mutated proteins, which must then be presented via cell surface associated class I HLA molecules (HLA-I). T cell receptors (TCRs) interact with particular neoepitope-HLA complexes such that the therapeutic target definition comprises both the neoepitope sequence and HLA-I subtype upon which it is presented. Neoepitopes are generally 8-11 amino acids in length, and as a result a single amino acid substitution may be presented within 38 possible neoepitopes (i.e., eight (8)-mers, nine (9)-mers, ten (10)-mers, and eleven (11)-mers). In addition, HLA-I molecules are highly polymorphic with more than 1000 documented variants that each has the capacity to bind a distinct subset of peptides. Consequently, the number of potential neoepitope-HLA targets increases quickly and even if development was focused on neoepitopes derived from the most common 50 cancer neoantigens across the most prevalent 15 HLA alleles, >28,000 neoepitope-HLA pairs could be formed. However, not all of these combinations are therapeutically relevant because even if a neoepitope can bind an HLA molecule, there is no guarantee that the specific neoepitope would be presented within the biological context of a tumor cell. Most of these alleles are very rare and would not constitute a strong candidate for drug development given the rarity of patients harboring both the neoantigen mutation and these rare alleles.

Therefore, the top 15 most common HLAs were tested for their interaction with each of the 38 neoepitopes, making 570 possible HLA-neoepitope combinations that can be derived from a single neoantigen. In addition, to perform a comprehensive analysis of neoepitope targets across all clinically relevant neoantigens in terms of prevalence and clinical development, the number of combinations grows to greater than 28,000 (assuming ˜50 clinically relevant neoantigen targets), which is a significantly large number of combinations to evaluate. However, not all of these combinations are therapeutically relevant because even if a neoepitope can bind an HLA molecule, there is no guarantee that the specific neoepitope would be available to bind within the biological context of a tumor cell.

One approach to quickly select biologically relevant target neoepitope-HLA pairs from the 28,000 possible combinations is using neoepitope prediction algorithms. Several different algorithms have been developed to rank neoepitope-HLA pairs for a given neoantigen. However, these algorithms are unable to accurately predict presentation of a neoepitope by HLA. Despite the significant advances in the accuracy of neoepitope-HLA prediction algorithms, these methods cannot be solely relied on for identifying presented neoepitopes, which are the true targets of neoantigen targeted vaccine and T cell therapeutics.

Generation of neoepitopes is dependent on the antigen processing pathway (APP) as cancer neoantigens are degraded by the proteasome and the resulting peptides are imported into the ER where they are further processed by ER resident aminopeptidases before finally being loaded into an HLA molecule for presentation (Pishesha et al., A guide to antigen processing and presentation. Nat Rev Immunol. Apr. 13, 2022. doi: 10.1038/s41577-022-00707-2). As a result, it is possible that a synthetic neoepitope could bind an HLA molecule in vitro, but not be observed as a presented peptide within a cellular or in vivo context. A prime example is the description of a bi-specific antibody that targeted an A*02:01 restricted neoepitope of KRAS G12V (KLVVVGAVGV, SEQ ID NO: 191) (see Skora et al. 2015 Proc Natl Acad Sci USA 112(32):9967-9972; Douglass et al., Bispecific antibodies targeting mutant RAS neoantigens. Sci Immunol. 2021 Mar. 1; 6(57): eabd5515). This molecule demonstrated binding in vitro when HLA molecules were loaded with synthetic peptide, but failed to induce cell killing when tested in cell lines harboring the KRAS mutation. For this reason, direct identification of presented peptides through mass spectrometry (MS) based immunopeptidomics approaches is a key aspect of neoantigen target validation. For example, a targeted MS approach was used to provide further evidence that the aforementioned KRAS A*02:01 neoepitope is not presented in a cellular context (Choi et al., Systematic discovery and validation of T cell targets directed against oncogenic KRAS mutations. Cell Rep Methods. 2021; 1(5): 100084). While extremely sensitive, such targeted MS assays require heavy isotope labeled peptides for each potential neoepitope as well as cell lines expressing the cancer neoantigen of interest. Due to these limitations, targeted MS assays are typically employed to evaluate a small number of cancer neoantigens within a given study.

To address this limitation, a pipeline is presented herein for comprehensive discovery and validation of neoepitope-HLA pairs presented on the surface of cells. The first step in this process was to perform a clinico-genomics analysis of all known neoantigens to identify neoantigens that are of high value as clinical targets. In this analysis, prevalence within and across indications was evaluated as well as clinical developability. From this analysis, 48 neoantigens were selected.

The next step was to develop a high throughput HLA binding assay to screen neoepitope-HLA combinations presented on the surface of cells (e.g., 27,360 combinations for 48 neoantigens, 38 neoepitope/neoantigen, and 15 HLA alleles, or 26,220 neoepitope-HLA combinations for 47 neoantigens as shown in Table 3, 38 neoepitope/neoantigen, and 15 HLA alleles as shown in Table 4). Using a clinico-genomics approach 47 common cancer point mutations and 15 prevalent HLA-I alleles were selected to enable characterization of the neoepitope landscape for clinically actionable neoantigen targets. A novel high throughput HLA binding assay was then used to experimentally screen in vitro stabilization for all potential 24,149 neoepitope-HLA combinations to identify 587 stable complexes (Darwish et al., Protein Sci. 2021; 30(6): 1169-1183; Rodenko et al., Generation of peptide-MHC class I complexes through UV-mediated ligand exchange. Nat Protoc. 2006; 1(3):1120-32). To understand the complementarity of in vitro and in silico identification of neoepitope-HLA complexes, results from the high throughput binding assay were supplemented by an additional subset of neoepitope-HLA combinations that did not form stable complexes in vitro, but were predicted to bind by NetMHCpan4.0 (Jurtz et al., NetMHCpan-4.0: Improved Peptide-MHC Class I Interaction Predictions Integrating Eluted Ligand and Peptide Binding Affinity Data. J Immunol. 2017; 199(9):3360-3368). The resulting neoepitope-HLA pairs were assayed for presentation using both untargeted and targeted mass spectrometry analysis of HLA-I monoallelic cell lines that simultaneously expressed ˜25 amino acid segments corresponding to each of the 47 cancer neoantigens. This analysis produced a list of 84 neoepitope-HLA pairs representing, to the best of the knowledge, the broadest list of experimentally validated presented neoepitopes. Lastly, the characterization of the therapeutic potential for these targets utilized TCRs discovered using the well-established Multiplex Identification of T cell Receptor Antigen (MIRA) assay in a parallel therapeutic discovery effort to demonstrate mutant-selective T cell activation and killing of cells expressing either an A*02:01 FLT3 D835Y or an A*11:01 PIK3CA E454K neoepitope. For example, in one exemplary experiment, through the development of a high-throughput peptide-HLA binding assay, the binding of 26,790 peptide-HLA combinations was characterized resulting in 643 stable complexes. These results were used to build sensitive targeted mass spectrometry assays to validate neoepitope presentation on 15 monoallelic cell lines containing a construct encoding for 47 cancer neoantigens. This analysis detected 79 unique peptide-HLA pairs derived from 34 shared cancer neoantigens and presented across 12 HLA alleles. Together these data represent a valuable resource of therapeutically relevant neoepitopes and the HLA context in which they can be targeted.

### Materials and Methods

### Clinico-Genomics Analysis of Shared Cancer Neoantigens

Prevalence data for common cancer mutations (SNVs and indels) were obtained from the Cancer Hotspots database (World Wide Web site at cancerhotspots.org; see Chang et al., 2018 Cancer Discov. 8(2): 174-183) and cross-referenced with TCGA data obtained from the cBioPortal for Cancer Genomics (World Wide Web site at cbioportal.org). Prevalence data for common HLA alleles from the general population were obtained from the Allele Frequency Net Database (World Wide Web site at allelefrequencies.net) and from HLA typing of >8,000 TCGA cases. From these data sets, the 48 most common cancer mutations were determined (based on prevalence per cancer type), and the 48 most common HLA-I alleles were determined. Additional ranking of these mutations was performed that considered the overall prevalence of each cancer type, and whether a neoantigen-specific therapy could be readily developed in a clinical setting.

### Predicted Neoepitope Landscape Analysis

After translating mutations to peptide sequences, neoepitope-HLA binding predictions were generated using NetMHCpan-4.0 (Jurtz et al., NetMHCpan-4.0: Improved Peptide-MHC Class I Interaction Predictions Integrating Eluted Ligand and Peptide Binding Affinity Data. J Immunol. 2017; 199(9): 3360-3368) on all combinations of 8, 9, 10, 11, 12 or 13-mer peptides derived from the 47 cancer neoantigens combined with the 15 most prevalent HLA alleles. Both binding affinity (BA) and eluted ligand (EL) predictions were obtained, which were then used for downstream analysis. Predicted neoepitopes were defined as neoepitope-HLA combinations with mutant EL percentile rank<2.

### Protein Expression and Purification

Recombinant HLA and B2M were over-expressed in E. coli, purified from inclusion bodies, and stored in denaturing buffer (6 M Guanidine HCl, 25 mM Tris, pH 8) at −80° C. as described previously (Darwish et al., Protein Sci. 2021; 30(6): 1169-1183). Briefly, B2M and HLA biomass pellets were re-suspended in lysis buffer (PBS+1% Triton X-114) at 5 mL/g and homogenized twice in a microfluidizer at 1000 bar. The homogenized suspension was spun at 30000 g for 20 min in an ultracentrifuge. The pellets were collected and washed with 500 ml of 0.5% Triton X-114 in PBS. Collected samples were then centrifuged at 30000 g for 20 min. The pellet was collected again and washed as described above. The purified inclusion bodies were dissolved in denaturing buffer (20 mM MES, pH 6.0, 6 M Guanidine) at a concentration of 10 ml/g and stirred at 4° ° C. overnight. The dissolved pellet was centrifuged at 40000 g for 60 min and the supernatant was collected and filtered through a 0.22 mm filter. The concentration was determined by UV-vis at 280 nm using the protein's extinction coefficient. Samples were then snap-frozen and stored at −80° C. prior to generation of complexes.

### HLA-I-Peptide Refold, Biotinylation, and Purification

Conditional HLA-I complexes were generated in a 5L refold reactions in refold buffer (100 mM Tris, pH 8.0, 400 mM L-Arginine, 2 mM EDTA) as described previously (Darwish et al., 2021). Briefly, the refold reaction consisted of the conditional HLA-I ligand peptide containing a non-natural UV cleavable amino acid (0.01 mM), oxidized and reduced glutathione (0.5 mM and 4.0 mM, respectively), recombinant HLA (0.03 mg/ml) and β2M (0.01 mg/ml). The refold mixture was stirred for 3-5 days at 4° C., filtered through a 0.22 μm filter, and concentrated and buffer exchanged by tangential flow filtration (TFF) (Millipore P2C010C01) into 25 mM Tris pH 7.5. The concentrated and refolded HLA-I complex was then biotinylated through the addition of BirA [1:50 (wt:wt) for the enzyme: HLA-I ratio], 100 mM ATP and 10×reaction buffer (100 mM MgOAc, 0.5 mM biotin) for an incubation period of 2 hr at room temp. The sample was dialyzed and analyzed by LC/MS to quantify biotinylation. The biotinylated HLA-I complex was purified by anion exchange chromatography using a 1 ml HiTrap Q HP column on an AKTA Avant FPLC. The column was equilibrated with 10 column volumes (CV) of 25 mM Tris-HCl pH 7.5 at a flow rate of 5 ml/min. The refolded peptide-HLA-I sample was loaded on the column at a 5 ml/min flow rate and eluted using 0-60% 2.5 mM TrisHCI, pH 7.5, 1 M NaCl gradient over 30 CV. Fractions across the eluted peak were run on SDS-PAGE, and fractions containing both B2M and HLA bands were pooled. Pooled fractions were buffer-exchanged into storage buffer (25 mM Tris HCl, pH 8.0, 150 mM NaCl). Protein concentration was determined by UV absorbance at 280 nm, and samples were snap-frozen and stored at −80° C.

### Peptides Synthesis for In Vitro Binding Assay

Peptides for the binding screen were synthesized by JPT Peptide Technologies GmbH (Germany) and purified to >70% purity by HPLC. Peptides were dissolved in ethylene glycol (Sigma) at 1 mg/mL and stored at −80° ° C. in Matrix 1.0 mL 2D screw cap tubes (Thermo Fisher Scientific). UV-cleavable peptides were synthesized with 3-amino-3-(2-nitrophenyl)propionic acid by Elim Biopharm and purified by HPLC to >70% purity.

### Automated High Throughput Neoepitope Exchange

Peptides were diluted to 10 μM in 25 mM TRIS pH 8.0, 150 mM NaCl, 4 mM EDTA, 4.35% ethylene glycol, in 96 deep well plates (VWR) using a Biomek i5 automated liquid handler (Beckman Coulter). The peptide-buffer mixtures were dispensed and reformatted into 384 well plates (Labcyte) at a volume of 47.5 μl per well, resulting in identical plates of up to 352 unique Neoepitopes for screening against each of the 15 HLA alleles. The first two columns of the plate were reserved for controls. HLA A*02:01 with and without exchange peptide were included on each plate as positive and negative controls for exchange, respectively. The well characterized HLA A*02:01 specific viral epitope, CMV pp65 peptide (NLVPMVATV, SEQ ID NO: 76, Elim Biopharm), was plated in quadruplicate, as a positive control for peptide exchange. Negative controls for exchange included wells to which no peptide was added, and instead received ethylene glycol only during the peptide dilution step. Negative control wells for the HLA allele being screened were plated in octuplicate.

Using a Mantis liquid handler (Formulatrix), 2.5 ul of 0.1 mg/ml UV peptide-HLA complexes were added to each well with one HLA allele screened for binding per plate. Positive control wells received HLA A*02:01, and negative control wells received either HLA A*02:01 or the HLA allele specific to the plate. The resultant peptide exchange reaction mixtures contained 10 μM peptide, 0.1 μM UV-HLA complex, and 5% ethylene glycol v/v.

The peptide exchange protocol was adapted from a method previously described (Rodenko et al., Nat Protoc. 2006; 1(3): 1120-1132) by decreasing the UV exposure time and adding an incubation step after UV exposure. Plates containing the peptide exchange reaction mixtures were incubated under UV lamps (UVP 3UV Lamp, Analytik Jena) for 25 min using one lamp per plate. Plates were then sealed and incubated for 18 hours at room temperature.

### TR-FRET Assay

To determine HLA binders, a TR-FRET assay was developed that provides a signal only when the B2M and HLA complex are in close proximity. This assay uses an antibody against B2M that contains a TR-FRET donor (anti-B2M-donor) and streptavidin, which binds to the biotinylated HLA component of the complex, labeled with a TR-FRET acceptor (Streptavidin-Allophycocyanin (SA)-acceptor). If B2M and HLA are complexed together than anti-B2M donor and SA-acceptor will be close in solution resulting in a TR-FRET signal. In contrast, if the complex falls apart these reagents will be evenly distributed and there will be no TR-FRET signal.

A 384 well source plate (Echo Qualified 384-Well Polypropylene 2.0 Plus Microplate, Labcyte PPL-0200) containing UV-exchanged HLA/peptide complex was incubated at 37° ° C. overnight. The plate was equilibrated at RT for 1 hour followed by centrifugation. Each well of the source plate was dispensed four times at various volumes (160 nL, 80 nL, 40 nL and 20 nL) with an automated acoustic dispenser (Echo 550, Labcyte) into the back filled wells (with assay buffer) of the destination plate (MAKO 1536 well white solid bottom, Aurora Microplates, Whitefish, MT) for a total volume of 4 μL/well (2 μL diluted samples and 2 μL of reagent mix) with final sample concentrations at 10, 5, 2.5 and 1.25 nM. In brief, 1.8 μL per well of assay diluent (PBS, 0.5% BSA+0.05% Tween 20+10 PPM Proclin, Genentech, Inc) was added to the 1536-well destination plate by a Multidrop™ Combi nL Dispenser (Thermo Fisher Scientific, Waltham, MA). Then 200 nL of 5 μg/mL of HLA complex sample were dispensed from the Echo qualified 384-well source plate (Beckman Coulter Life Sciences, Indianapolis, IN)) into the destination plate by an Echo 550 acoustic liquid dispenser (Beckman Coulter Life Sciences, Indianapolis, IN). After centrifugation for three minutes, two μL of master mix donor at 2 nM (Europium mouse anti-human β2-microglobulin (β2M), Biolegend, San Diego, CA, custom labeled by Perkin Elmer, Waltham, MA) and acceptor at 40 nM (SureLight Allophycocyanin conjugated Streptavidin (SA-APC), PerkinElmer, Waltham, MA) in assay diluent were dispensed into each well of the destination plate with the Multidrop™ Combi nL dispenser. The destination plate was then centrifuged and incubated at room temperature for one hour, the TR-FRET signals were recorded using PHERAstar FSX plate reader (BMG Labtech, Cary, NC) equipped with HTRF Module (Eu donor excitation 337 nm, Eu donor emission 615 nm; APC acceptor emission 660/20 nm, e.g., 665 nm). The TR-FRET raw signals were expressed as ratios of relative fluorescent unit (RFU ratio=(RFU[665 nm]/RFU[615 nm]×104). The detection window was calculated by subtracting the background signal from the assay mix in the absence of HLA/peptide complex. For ranking the binders, a double normalization was applied to obtain % DeltaF. DeltaF(%)={(RFU [Sample]−mean RFU [negative])/mean RFU[negative]} *100. The Robust Z score was calculated on the sample plate basis. For screening quality control, large-scale prepared positive control (A*02:01 with pp65) and negative control (A*02:01 only) were added to designed wells in each sample plate. The acceptance of the screen was determined by Z-factor calculated from the assay controls (Z-factor=1−{(3SD [positive]−3SD [negative])/(mean [positive]−mean [negative]}. Sample plates had Z-factor>0.4 were qualified for data process.

A peptide was determined to be a true binder based on a comparison to its predicted binding affinity (calculated using a binding prediction algorithm based on Andreatta M, and Nielsen, M. Gapped sequence alignment using artificial neural networks: application to the MHC class I system. Bioinformatics (2016) Feb 15;32(4):511-517. The peptide binders and the corresponding HLA alleles were identified using the TR-FRET and 2D LC/MS assays. The peptide sequences were submitted to the prediction algorithm, and each sequence was assigned a percentile rank. A percentile rank of 2 or less it is considered a binder.

### Engineering Monoallelic and Polyantigen Cassette-Expressing HMy2.CRI Cell Lines

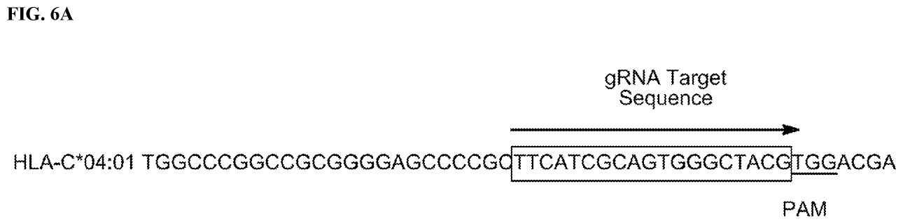

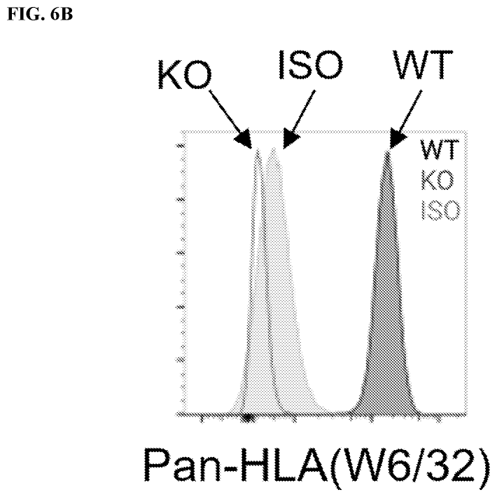

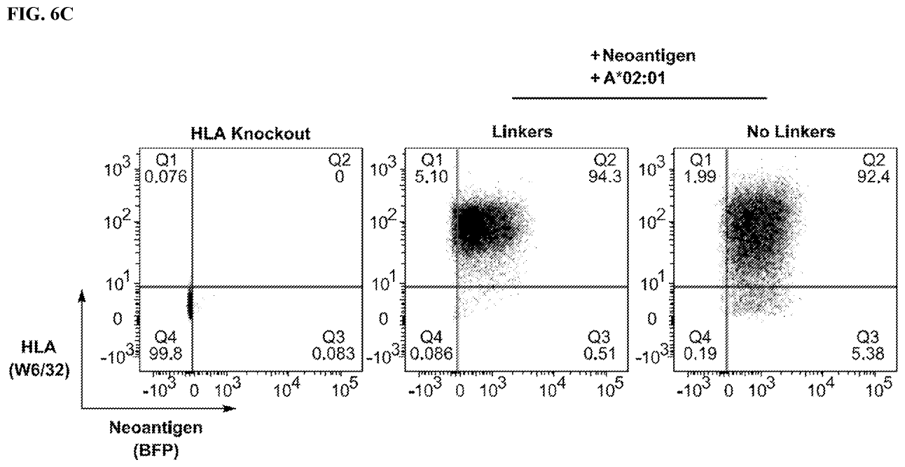

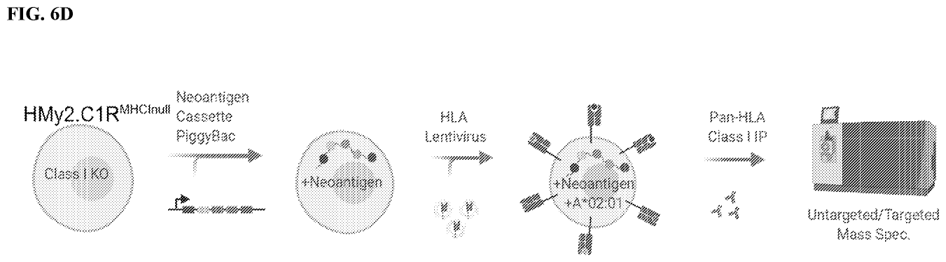

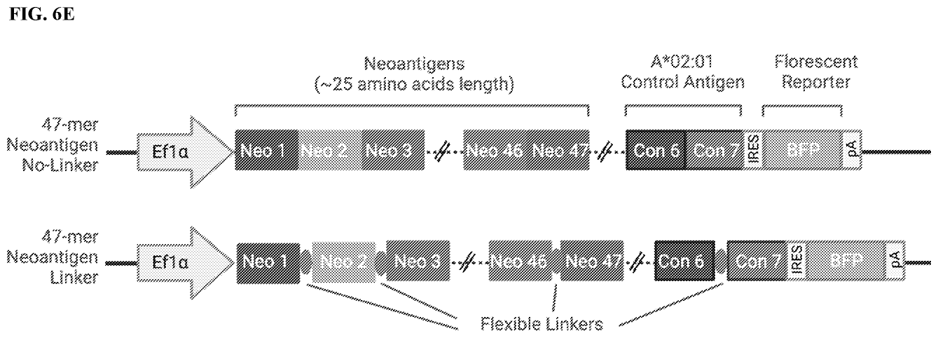

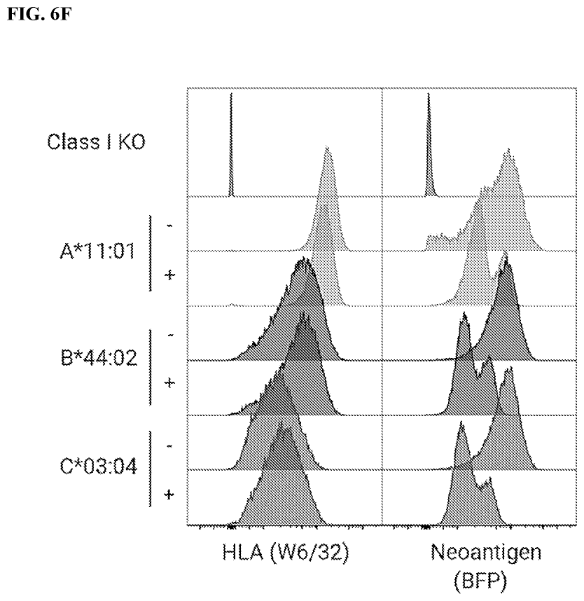

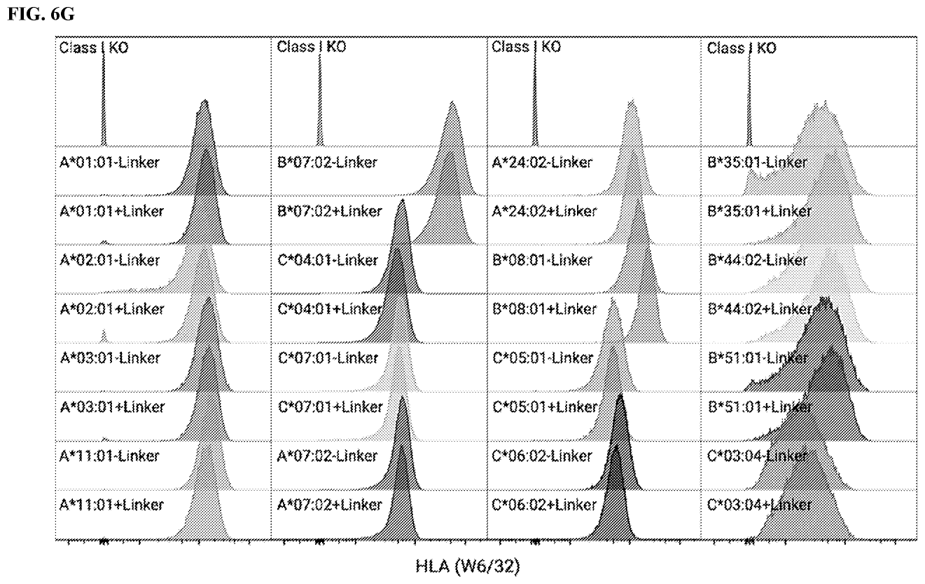

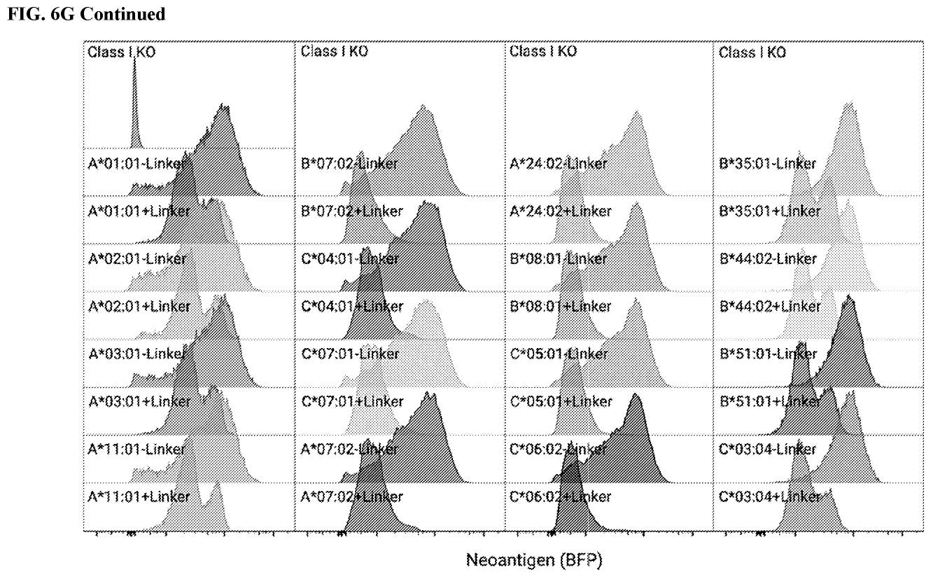

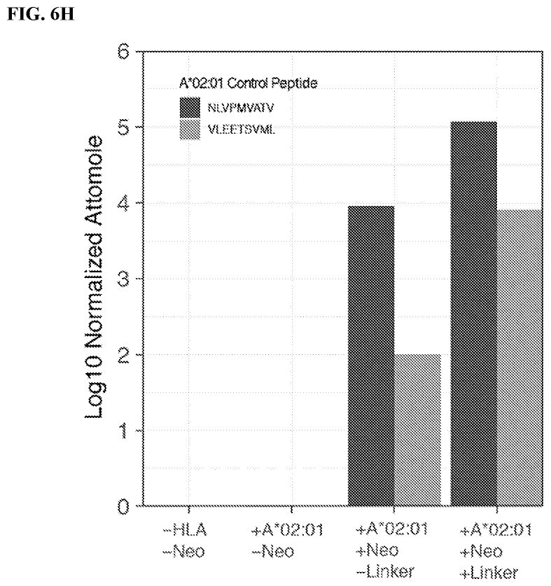

An effective HLA Class I knockout cell population was generated by CRISPR/Cas9 mediated gene disruption of the endogenous HLA-C locus in HMy2.C1R cells. Wild-type HMy2.C1R cells were electroporated with gRNA (Synthego; HLA-C-specific sgRNA sequence: TTCATCGCAGTGGGCTACG (SEQ ID NO: 74) (see FIG. 6A))/Cas9 (Invitrogen) RNPs using an Amaxa V system (program D-023). Following an expansion period, cells were stained with anti-pan-HLA (W6/32) antibody and antigen-negative cells were enriched via FACS (see FIG. 6B) to generate a class I knockout population.

HLA-I null HMy2.C1R cells were stably engineered with a piggyBac neoantigen expression plasmid system designed to co-express 47 shared cancer neoantigens and 7 HLA-A*02:01 control antigens. Briefly, neoantigens segments (˜25 amino acids each) were concatenated and converted to codon-optimized DNA segments (IDT), with or without a flexible linker separating most neoantigens sequences. The polyantigen cassettes were synthesized and cloned into a piggyBac transposon plasmid downstream of a constitutive human EFla and transcriptionally linked to an IRES-TagBFP2 reporter element. A separate hPGK promoter-driven puromycin resistance gene was included on the same vector for selection purposes. To generate cell lines stably expressing 47 neoantigens of interest, class I knockout HMy2.C1R cells were electroporated using a NEON (Invitrogen) electroporation device. Piggybac co-delivery of Piggybac plasmids (neoantigen cassettes) containing 47-mers with or without linkers was performed. Neoantigen cassette-containing cells were enriched by culture in 1 μg/mL puromycin (Gibco) and further purified by FACS enrichment of tagBFP positive cells. Two million C1R class I KO cells were re-suspended in 120 buffer R. 3.0 μg piggybac expression construct and 0.5 μg of piggbac transposase were added, and electroporated using the 100 μl Neon kit (buffer R, 1230 V, 20 ms, 3 pulses). Polyantigen-expressing cells were selected by culture in 1 μg/ml puromycin (Gibco) and further purified by FACS enrichment of the TagBFP2-positive population. Right after electroporation, cells were added to 5 ml antibiotic-free media in a 6-well plate. 7 days post electroporation, 1 μg/ml puromycin was added. After 3 days in puromycin, cells were analyzed and then BFP positive cells were sorted.

HLA expressing lentivirus was generated by co-transfection of an Efla-HLA expression construct along with the Delta8.9 and VSVG packaging plasmids into 293 cells using lipofectamine 2000 (Invitrogen) (Lipid:DNA=2:1). 72 hours post-transfection viral supernatant was harvested and filtered through a 0.45 μM filter and concentrated via LentiX concentrator reagent (Takara) following the manufacturers recommended protocol.

To generate neoantigen expressing monoallelic cell lines, linker or no-linker 47-mer-expressing class I knockout HMy2.C1R cells were transduced via spin infection. HLA expressing cells (biotin-W6/32) were purified via magnetic bead based enrichment (SA-MACS). HLA allele identification was confirmed via barcode sequencing and uniform expression of both the HLA allele and neoantigen cassette were confirmed via flow cytometry prior to analysis via mass spectrometry. Specifically, unique HLA-I allele ORFs, each with a distinct 19 bp DNA barcode, were cloned downstream of the human EFla promoter (Genscript) in a custom-modified pLenti6.3 backbone (ThermoFisher). Lentivirus was generated by lipofectamine 2000 (Invitrogen)-mediated co-transfection of HEK293T cells with individual lenti HLA expression constructs and packaging plasmids. 72 hours post-transfection, viral supernatant was harvested, filtered through a 0.45 μM filter, and concentrated via LentiX concentrator reagent (Takara) following the manufacturers recommended protocol. Linker or no-linker polyantigen-expressing HLA-I-null HMy2.C1R cells were transduced with HLA expression vectors via spin infection (800×g for 30 min at room temperature with 8 μg/ml polybrene). Transgenic HLA-expressing cells subsequently were purified via magnetic bead based enrichment (biotin-W6/32 Biolegend /SA-MACS). HLA allele identification was confirmed via barcode sequencing (Amplicon Primers: Fwd-CTCCCAGAGCCACCGTTACAC (SEQ ID NO: 192), Rev-GACTTAACGCGTCCTGGTTGC (SEQ ID NO: 193); sequencing primer: CTGGTTGCAGGCGTTTAGCGT; SEQ ID NO: 194) and uniform expression of both the HLA allele and polyantigen cassette were confirmed via flow cytometry (FIGS. 6B, 6F and 6G) prior to analysis via mass spectrometry.

In addition to HMy2.C1R cell, a similar result was detected by experiments using K562 cells.

### Antibody Coupling and Crosslinking

Pan-HLA Class I-specific antibody (clone W6/32) was coupled to Protein-A resin packed into AssayMAP Bravo compatible large capacity cartridges (PA-W 25 μL) (Agilent, Part number G5496-60018). The coupled antibodies were then crosslinked with 20 mM Dimethyl pimelimidate dihydrochloride (DMP) (Sigma-Aldrich, cat. D8388-250 MG) in 100 mM sodium borate crosslinking buffer at pH 9.0 immediately after the end of the coupling step. The impurities within the cartridges were washed away with simultaneous dispensing of 200 mM ethanolamine (Sigma-Aldrich, cat. E9508-100ML) pH 8.0 and deionized H₂O. The flow rates and other parameters of the affinity purification application within the AssayMAP Bravo software (VWorks) were used at the default settings. The antibody crosslinked Protein-A cartridges were stored in rack filled with TBS/0.025% sodium azide, sealed with parafilm, and kept at 4° C.

### Affinity Purification of HLA-Peptide Complexes

Engineered monoallelic cell pellets (500 million cells/sample) were lysed at 4° ° C. in 1% CHAPS (Roche Diagnostics, cat no. 10810126001) lysis buffer pH 8.0 containing 20 mM TRIS, 150 mM NaCl, one tablet of complete Protease Inhibitor Cocktail (Roche, cat. 4693159001) per 10 mL of the lysis buffer, and 0.2 mM phenylmethylsulfonyl fluoride (PMSF) (Sigma). The cell pellets were lysed with 2 mL of the lysis buffer by vortexing every 5 minutes for a total of 20 minutes at 4° C. The lysates were then transferred to LoBind tubes and centrifuged at 20,000 g at 4ºC for 20 minutes. The supernatants were then carefully transferred to 0.45 μm polyethersulfone filter (Pall, cat. MCPM45C68). The samples were then centrifuged at 7000 g at 4° ° C. for 30 minutes. The filtrate for each sample was carefully transferred to an AssayMAP Bravo compatible 96-well deep well plate making sure not to disturb any particulates that might have settled at the bottom of the conical tube. The deep well plate containing HLA-peptide complexes was transferred to the AssayMAP Bravo sample loading platform for automated dispensing of the samples through the W6/32 crosslinked Protein-A cartridges. The cartridges were primed and equilibrated with 20 mM Tris pH 8.0 and 150 mM NaCl in water. The sample impurities within the cartridges were washed away with automated dispensing of 20 mM Tris pH 8.0 and 400 mM NaCl in water followed by final wash with 20 mM Tris pH 8.0 in water. The antibody-bound HLA-peptide complexes were eluted with 0.1 M acetic acid in 0.1% trifluoroacetic acid (TFA). The flow rates and wash cycles were used at the default settings.

The eluates were transferred to ultra-low adsorption ProteoSave autosampler vials (AMR Incorporated cat. PSVial 100) and dried in a speed vacuum. The dried samples were then reconstituted in 100 μL 20 mM HEPES pH 8.0, reduced with 5 mM Dithiothreitol (DTT) (Thermofisher, cat. A39255) in the dark at 65° C. for 30 minutes, and alkylated with 15 mM Iodoacetamide (IAA) (Sigma, cat. I1149-5G) in the dark at RT for 30 minutes. The samples were then acidified with 50% TFA to drop their pH ˜ 3.0, vortexed, and centrifuged at 14,000 g at RT for 5 minutes to pellet any debris. The samples were then carefully transferred to 96 well PCR, Full Skirt, PolyPro plate (Eppendorf, Part number 30129300) and loaded on the AssayMAP Bravo platform for final clean up before injection into the mass spectrometer. Four C18 cartridges (Agilent, Part number 5190-6532) were used per sample. The cartridges were primed with 80% acetonitrile (ACN) 0.1% TFA and equilibrated with 0.1% TFA. The samples were then loaded through the cartridges, washed with 0.1% TFA, and eluted with 30% ACN 0.1% TFA. After drying the samples in a speed vacuum, the samples were reconstituted in 6 μL 0.1% formic acid (FA) 0.05% heptafluorobutyric acid (HFBA) (Thermo Fisher Scientific, cat. 25003).

### Untargeted Mass Spectrometry and Database Search

One-third of each sample was loaded into a 25 cm×75 μm ID, 1.6 μm C18 IonOpticks Aurora Series column (IonOpticks, Part Number AUR2-25075C18A) on a Thermo UltiMate 3000 high performance liquid chromatography (HPLC) system (Thermo Fisher Scientific) at a flow rate of 400 nL/min. Peptides were separated with a 90 minute gradient of 2% to 35% or 40% buffer B (98% ACN, 2% H2O, and 0.1% FA) at a flow rate of 300 nL/min. The gradient was further raised to 75% buffer B for 5 minutes and to 90% buffer B for 4 minutes at the same flow rate before final equilibration with 98% buffer A (98% H2O, 2% ACN, and 0.1% FA) and 2% buffer B for 10 minutes at a flow rate of 400 nL/min.

Peptide mass spectra were acquired using either Orbitrap Fusion Lumos or Orbitrap Eclipse Tribrid mass spectrometer (Thermo Fisher Scientific) with MS¹ Orbitrap resolution of 240000 and MS/MS fragmentation of the precursor ions by collision-induced dissociation (CID) followed by spectra acquisition at MS² Orbitrap resolution of 15000. All data-dependent acquisition (DDA) spectral raw files were searched in PEAKSOnline (Bioinformatics Solutions Inc.) against a Uniprot-derived Homo sapiens human proteome (downloaded Oct. 3, 2019) that contained appended concatenated sequences of 47 most common mutations flanked by 13-mer sequences on either end of each mutation with or without stretches of Glycine and Serine residue (GS) linkers along with sequences of blue fluorescence protein (BFP). Within PEAKSOnline, since HLA-peptides are non-tryptic the enzyme specificity was set as none, CID was selected as an activation method, and Orbitrap (Orbi-Orbi) was chosen as an instrument parameter. In-depth de novo assisted database search and quantification were performed with precursor mass error tolerance of 15 parts per million (ppm), fragment mass error tolerance of 0.02 Da, and missed cleavage allowance of 3. Carbamidomethylation (Cys+57.02) was set as a fixed modification whereas deamidation (Asn+0.98, Gln+0.98) and oxidation (Met+15.99) were set as variable post translational modification (PTM) allowing a maximum of 3 variable PTMs per peptide. Additional report filters included peptide spectral match (PSM) false discovery rate (FDR) of 1%, Proteins—10LgP≥20, and denovo only amino acid residue average local confidence (ALC) of 50%. For label free analysis, a new group was created for each sample and match between runs was performed with default parameters except retention time (RT) shift tolerance was set to 4 minutes and base sample was selected as “Average”. Output csv files were exported and further analyzed in R.

### Targeted Mass Spectrometry

Absolute quantification (AQUA) synthetic heavy peptides (8-11-mer) (Elim Biopharm) for all 47 mutation-derived neoantigens with TR-FRET RZ score≥5 (i.e. RobustzScore≥5) or predicted NetMHC % Rank≤2 (for a subset of mutations) were reconstituted in 30% ACN 0.1% FA. Dimethyl sulfoxide (DMSO) was added for peptides that were not readily soluble in 30% ACN 0.1% FA. A working solution of 25 μM was made for each AQUA peptide from which allele-specific mastermix was made at 25 pmol/peptide. The peptides were reduced/alkylated and cleaned up with C18 cartridges on AssayMAP Bravo. After drying, the peptides were reconstituted in 0.1% FA 0.05% HFBA at 100 fmol/peptide. For each allele-specific assay the intact modified mass was calculated for each peptide in that assay using TomahaqCompanion software (Rose et al., J Proteome Res. 2019; 18(2):594-605) which was then used to build an inclusion list mass spectrometry method for a scouting run to get the RT and mass-to-charge (m/z) of each target peptide. 1 μL of each assay was injected into the IonOpticks C18 column and sprayed into the mass spectrometer for a 125 minutes run as described above and the raw files were imported and analyzed in Skyline (64-bit, 19.1.0.193) to select appropriate charge for each peptide. A mass list table was built for each assay where 4 minutes RT window was created on both sides of the RT for each target peptide which was then imported into Xcalibur instrument method application and saved as an allele-specific parallel reaction monitoring (PRM) method. For both Fusion Lumos and Eclipse instruments MS1 was acquired at Orbitrap resolution of 240000 with a maximum injection time of 50 ms followed by quadrupole isolation window of 1.2 m/z, CID fragmentation of parent ions, maximum injection time of 300 ms, and MS² acquisition at Orbitrap resolution of 60000. For Eclipse acquisition MS¹ and MS² AGC targets were set at 250% and 400% respectively. One-third of each monoallelic sample was spiked with 100 fmol of corresponding AQUA mastermix and injected into the mass spectrometer using the same HPLC set up as described above. Raw PRM data were imported and analyzed in Skyline in allele-specific manner. The ratios of the light peptides to their heavy counterparts across samples were exported as csv files and further analyzed in R. For each neoepitope, background signal detected in the synthetic peptide only analysis was subtracted from endogenous peptide signal before calculation of a final attomole amount.

For KRAS wild type (WT) and G12C/D/V mutant copy number presentation quantification in dox-inducible C1R A*11:01 KRAS full length (FL) cell lines and C1R A*11:01 47-neo sample recombinant heavy isotope-coded peptide MHC (hipMHC) (Stopfer et al., Multiplexed relative and absolute quantitative immunopeptidomics reveals MHC I repertoire alterations induced by CDK4/6 inhibition. Nat Commun. 2020; 11(1):2760) monomers were made in-house for A*11:01 allele and KRAS WT/G12C/D/V (9- and 10-mer per target). These monomers were spiked at 1 pmol per 500 million cell lysate (KRAS FL samples) or 4.7 pmol per 500 million cell lysate (47-neo sample) immediately before the pan HLA Class I affinity purification step. Similar to shared neoantigen samples, an inclusion method and a 125 minutes PRM method were developed for A*11:01 KRAS FL samples where only 8 AQUA peptides were in the hipMHC assay mix. The raw data were analyzed as described above with additional steps where on-column AQUA peptide concentration and input cell count were taken into consideration to calculate antigen copies per cell.

For absolute quantification of total KRAS WT and G12C/D/V proteins in dox-inducible C1R A*11:01 KRAS FL cell lines 20 millions cells per sample were lysed in 1 mL 8M Urea lysis buffer 20 mM HEPES pH 8.0. Yeast digest (25 μg) and 50 μg from each sample were spiked with 2.5 pmol KRAS quantification concatemer (QconCAT) polypeptide (Polyquant) generated by concatenation of heavy WT and select mutant RAS tryptic proteotypic peptides. Samples were reduced with 5 mM DTT in the dark at 56° C. for 10 minutes with shaking and alkylated with 15 mM IAA in the dark at RT for 15 minutes. Urea concentration across control and sample tubes were dropped to ˜2M with 20 mM HEPES pH 8.0 and were digested with 1 μg sequencing grade trypsin (Promega) overnight at 37° C. in a nutator. Next day, trypsinization was quenched with 50% TFA and samples were cleaned up on C18 cartridges, dried, and reconstituted at 100 fmol/μL (yeast digest control) or 50 fmol/L (samples) in 0.1% FA. The digested samples were run on Fusion Lumos mass spectrometer with a 65 minutes PRM method specific for RAS tryptic peptides present on QconCAT polypeptide. Data were analyzed on skyline and absolute quantification of each of the target KRAS peptides was calculated.

### TCR Discovery

376 predicted and mass-spec identified neoantigen-derived peptides were synthesized (GenScript) and each was added to 6 of 11 peptide pools such that each neoepitope (or group of similar neoepitopes) occupied a unique combination of 6 pools (Klinger et al. 2015 PLOS One. 10(10):e0141561). CD8+ T cells were isolated (StemCell) from healthy human donor leukopaks and expanded either on anti-CD3 coated plates (+anti-CD28/IL-2, BioLegend), or in the presence of matched donor-derived monocyte-derived dendritic cells (Wölfl and Greenberg. 2014 Nat Protoc. 9(4):950-966) and a pool of all 376 neoepitopes. At day 10-15, T cells were recovered, supplemented with 1 of the 11 neoepitope pools, incubated 8-14 hours, enriched (Miltenyi) and then sorted using an anti-CD137 antibody (BioLegend). Sorted cells were then subjected either to immunoSEQ or pairSEQ (Adaptive Biotechnologies) to identify TCRβ sequences displaying neoepitope-specific responsiveness and to associate TCRB with TCRa sequences in parallel, respectively. TCR sequences were encoded in pcDNA vectors as a single open reading frame, in the form of the full TCRβ sequence followed by an RAKR motif and porcine teschovirus 2a cleavage peptide with the full TCRA sequence following in frame. TCR-encoding pcDNA vectors were then used as templates to generate TCR-encoding in vitro transcribed RNA (ivtRNA; mMessage mMachine, ThermoFisher) for electroporation of primary human T cells.

### TCR Reactivity Assays

CD8+ cells were enriched from human PBMCs with EasySep Human CD8+T Cell Isolation Kit (Stemcell) and stimulated with 5 μg/mL Ultra-LEAF anti-human CD3 (Biolegend) and 2.5 μg/mL Ultra-LEAF anti-human CD28 (Biolegend). Cells were cultured in the presence of 20 ng/mL recombinant human IL-2 for 6 days. Human expanded CD8+T cells were transfected with FLT3-p.D835Y-specific or PIK3CA-p.E545K-specific TCR RNA using a Lonza 4D-Nucleofector, P3 primary cell 4D-nucleofector kit, program EO-115 (Lonza). RNA was purchased from Trilink or in vitro transcribed. FLT3-p.D835Y-specific TCRs were co-cultured overnight with HLA-A*02:01-expressing T2 cells pulsed with YIMSDSNYV (SEQ ID NO: 116) or HLA-A*02:01-expressing K562 cells transfected with a construct encoding the mutant or wild-type sequence. K562 cells were transfected using a Lonza 4D-Nucleofector, SF cell line 4D-nucleofector kit, program FF-120 (Lonza). To determine specific cell lysis, an equal mixture of transfected HLA-A*02:01+K562 cells and untransfected cellTrace FarRed (Thermofisher)-labeled HLA-A*02:01+K562 cells were co-cultured overnight with T cells at a 2:1 E:T ratio. % Specific Cell Lysis=(P_{mock-transfected T cells}−P_{TCR-transfected T cells})/(P_{mock-transfected T cells}))×100, where P is the proportion of transfected K562 targets relative to an untransfected K562 cells, as measured by flow cytometry. CD137 expression on CD8+ T cells was assessed after an overnight co-culture with an anti-CD137 PE antibody (BD Biosciences). TNF and IFNg levels were determined with Cytometric Bead Array (BD Biosciences).

PIK3CA-p.E545K-specific TCRs were co-cultured overnight with HLA-A*11:01-expressing K562 cells pulsed with STRDPLSEITK (SEQ ID NO: 169) or transfected with a construct encoding the mutant or wild-type sequence. Equal mixtures of cellTrace Far Red-labeled HLA-A*11:01+K562 cells were added to each well. T cell response to PIK3CA-presenting K562 cells was assessed as above.

### Results and Discussion

### Clinico-Genomics Analysis of Shared Cancer Neoantigens

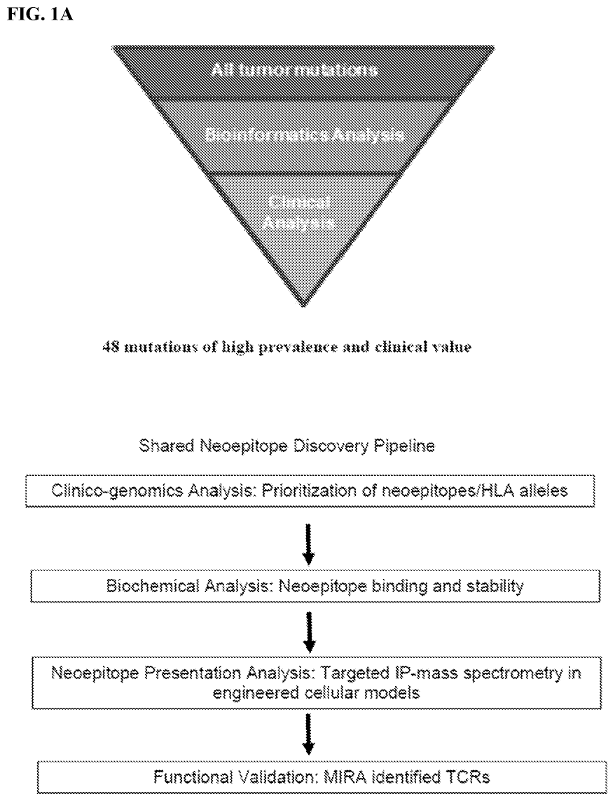

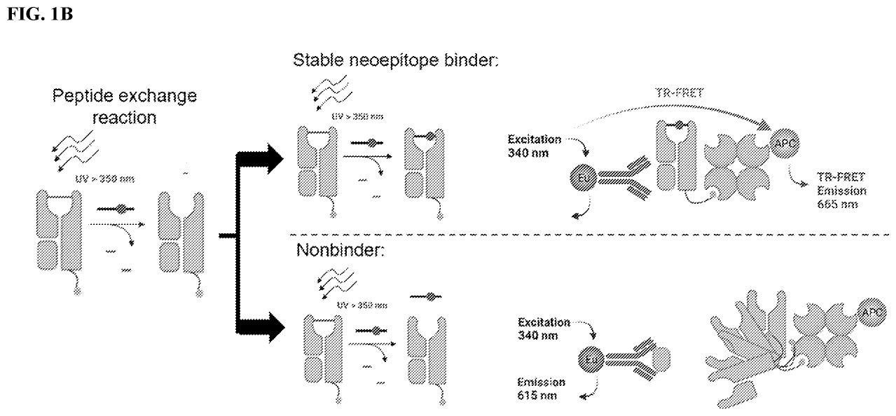

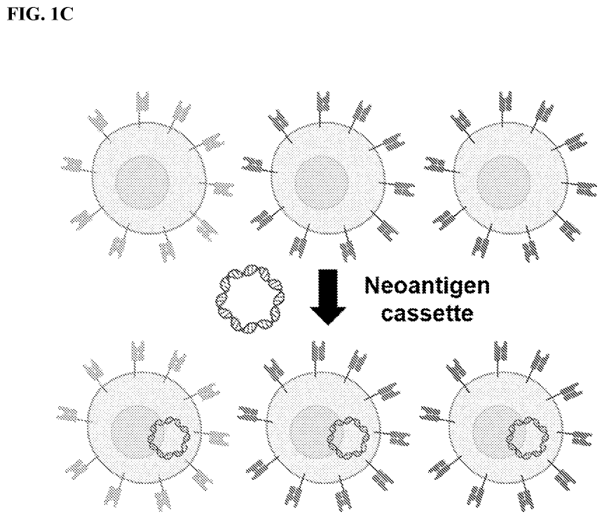

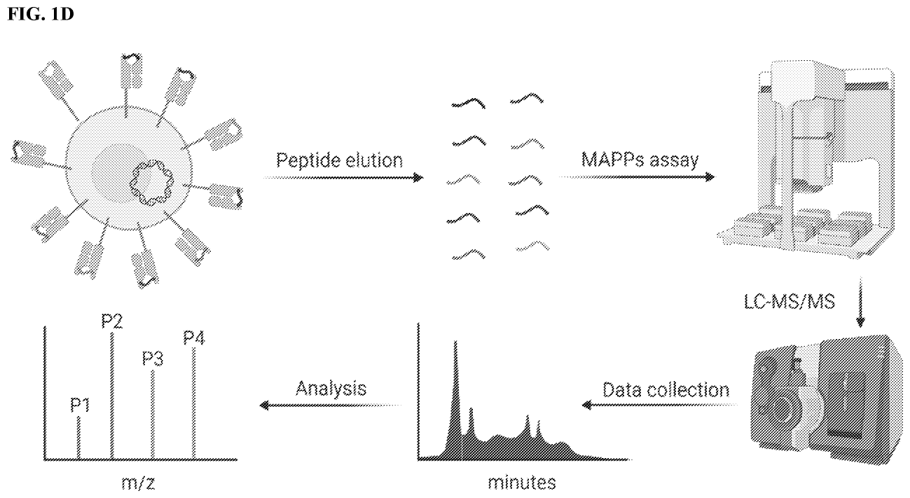

The schematic highlighted in FIG. 1A provides a high level overview of the workflow developed to enable neoepitope discovery in this study. The first step in this process was to perform a clinico-genomics analysis of all known neoantigens to identify neoantigens that are of high value as clinical targets. In this exemplary analysis, the most common recurrent point mutations were identified across cancer types from a large compendia of tumor and normal sequencing data (see, e.g., Chang et al., Accelerating Discovery of Functional Mutant Alleles in Cancer. Cancer Discov. 2018; 8(2): 174-183), filtered at a per-indication case prevalence of 2%. Gene fusions were excluded due to the high diversity of their breakpoints and resulting coding sequences, leading to a list of 37 shared cancer neoantigens (Table 3). Separately, the most common HLA-I alleles were identified across human populations, filtered at a carrier frequency of 10%. It was also verified that these alleles were present at equivalent frequencies in patients from The Cancer Genome Atlas (TCGA). This additional filtering led to a list of 15 HLA alleles. Co-prevalence of these shared cancer neoantigens and HLA-I alleles in the TCGA data was analyzed to ensure against biased co-expression of recurrent mutated genes and common HLA-I variants. Co-prevalence of each shared neoantigen was found to match expected values that were calculated as the product of the individual neoantigen and HLA-I allele prevalence. Together, these 37 neoantigens and 15 HLA alleles provided the foundation for development of the current platform.

TABLE 3
Exemplary shared neoantigens
A list of 47 cancer neoantigens:

| | | |
|---|---|---|
| KRAS G12C | KRAS G12R | TP53 R273H |
| KRAS G12D | PIK3CA E545K | TP53 R273L |
| KRAS G12V | PIK3CA H1047L | TP53 R282W |
| KRAS G13D | PIK3CA H1047R | CALR fs |
| KRAS G12A | ERBB2 S310F | ESR1 K303R |
| KRAS G12S | BRAF V600E | FLT3 D835Y |
| KRAS G13C | BRAF V600M | GNA11/GNAQ Q209L |
| JAK2 V617F | DNMT3A R882H | GNA11/GNAQ Q209P |
| NRAS Q61K | FGFR3 S249C | IDH1 R132C |
| NRAS Q61R | PIK3CA E542K | IDH1 R132G |
| EGFR E746_A750de | MYD88 L265P | IDH1 R132H |
| EGFR G719A | PTEN R130G | IDH2 R140Q |
| EGFR L858R | PTEN R130Q | SF3B1 R625C |
| EGFR T790M | TP53 R175H | SF3B1 R625H |
| EGFR C797S | TP53 R248Q | HRAS Q61R |
| EGFR T790M_C797S | TP53 R273C | |

A list of 37 shared cancer neoantigens after filtering:

| Peptide | Type | Map | Antigen | Linker |
|---|---|---|---|---|
| ELAGIGILTV | Control | MART-1 | Control | |
| (SEQ ID NO: 75) | | | | |
| NLVPMVATV | Control | pp65 | Control | |
| (SEQ ID NO: 76) | | | | |
| ATVQGQNLK | Control | pp65 | Control | |
| (SEQ ID NO: 77) | | | | |
| ELAGIGILT | Control | MART-1 | Control | |
| (SEQ ID NO: 78) | | | | |
| VLEETSVML | Control | IE-1 | Control | |
| (SEQ ID NO: 79) | | | | |
| FLYGSKTFI | Tag | BFP | Control | |
| (SEQ ID NO: 80) | | | | |
| GTVDNHHFK | Tag | BFP | Control | |
| (SEQ ID NO: 81) | | | | |
| TSNGPVMQK | Tag | BFP | Control | |
| (SEQ ID NO: 82) | | | | |
| IYNVKIRGVNF | Tag | BFP | Control | |
| (SEQ ID NO: 83) | | | | |
| VKIRGVNF | Tag | BFP | Control | |
| (SEQ ID NO: 84) | | | | |
| KPYEGTQTM | Tag | BFP | Control | |
| (SEQ ID NO: 85) | | | | |
| GPVMQKKTL | Tag | BFP | Control | |
| (SEQ ID NO: 86) | | | | |
| YRLERIKEA | Tag | BFP | Control | |
| (SEQ ID NO: 87) | | | | |
| ATSFLYGSK | Tag | BFP | Control | |
| (SEQ ID NO: 88) | | | | |
| GSHLIANAK | Tag | BFP | Control | |
| (SEQ ID NO: 89) | | | | |
| KPYEGTQT | Tag | BFP | Control | |
| (SEQ ID NO: 90) | | | | |
| GTVDNHHF | Tag | BFP | Control | |
| (SEQ ID NO: 91) | | | | |
| MEGTVDNHHF | Tag | BFP | Control | |
| (SEQ ID NO: 92) | | | | |
| KMDWIFHTA | Junction | PIK3CA H1047L-ERBB2 S310F | Junction | No |
| (SEQ ID NO: 93) | | | | |
| ATDFVKLKK | Junction | IDH1 R132G-IDH2 R140Q | Junction | No |
| (SEQ ID NO: 94) | | | | |
| RATDFVKLK | Junction | IDH1 R132G-IDH2 R140Q | Junction | No |
| (SEQ ID NO: 95) | | | | |
| SYVKVLHSI | Junction | MAGE A3-NYESO 1 | Junction | No |
| (SEQ ID NO: 96) | | | | |
| SPARPGKVV | Junction | CALR fs-FLT3 D835Y | Junction | No |
| (SEQ ID NO: 97) | | | | |
| DQYMRTIL | Junction | NRAS/HRAS Q61R-EGFR G719A | Junction | No |
| (SEQ ID NO: 98) | | | | |
| VTDLTVKI | Junction | ERBB2 S10F-BRAF V600m | Junction | No |
| (SEQ ID NO: 99) | | | | |
| RATDFVRLV | Junction | IDH1 R132G-IDH2 R140Q | Junction | No |
| (SEQ ID NO: 100) | | | | |
| LTIQLIQEQLK | Junction | KRAS G12S-PIK3CA E542K | Junction | No |
| (SEQ ID NO: 101) | | | | |
| LTIQLIQNK | Junction | KRAS G13C-PIK3CA E545K | Junction | No |
| (SEQ ID NO: 102) | | | | |
| AMTEYKL VVV | Junction | SF3B1 R625C-KRAS G12C | Junction | No |
| (SEQ ID NO: 103) | | | | |
| LLHRGNYMC | Junction | PTEN R130Q-TP53 R248Q | Junction | No |
| (SEQ ID NO: 104) | | | | |
| PARPGKVV | Junction | CALR fs-FLT3 D835Y | Junction | No |
| (SEQ ID NO: 105) | | | | |
| YLLHRGNYM | Junction | PTEN R130Q-TP53 248Q | Junction | No |
| (SEQ ID NO: 106) | | | | |
| YVKVLHSI | Junction | Mart1-MAGE A3 | Junction | No |
| (SEQ ID NO: 107) | | | | |
| YVVRGNAL | Junction | FLT3 D835Y-GNAQ Q209L | Junction | No |
| (SEQ ID NO: 108) | | | | |
| SGSGSLSHK | Junction | Linker-JAK2 V617F | Junction | Yes |
| (SEQ ID NO: 109) | | | | |
| GSGGEALEY | Junction | Linker-PIK3CA H1047R | Junction | Yes |
| (SEQ ID NO: 110) | | | | |
| FPNHVAAIH | Junction | MYD88 L265P-PTEN R130Q | Junction | No |
| (SEQ ID NO: 111) | | | | |

### High Throughput TR-FRET Analysis of Neoepitope Exchange and Neoepitope-HLA Stability

T cell mediated neoantigen specific therapies require a neoepitope and HLA molecule to form a stable complex that can be presented on the surface of tumor cells. Computational algorithms can predict if a neoepitope will bind to a particular HLA, but experimental evidence increases confidence that potential neoepitopes were not missed due to inadequacies of a prediction model (e.g., under-trained on specific alleles or biased against particular amino acid residues). The next step in the neoepitope-HLA discovery process was applying a high-throughput (HTP) neoepitope binding screen across the 15 most prevalent HLA alleles for all neoepitopes derived from the prioritized list of 48 neoantigens, which yields a total of 27,360 neoepitope-HLA combinations that required screening. For these purposes, a custom high-throughput (HTP) TR-FRET binding assay was developed to employ conditional HLA complexes. A schematic of the assay is shown in FIG. 1B. In an exemplary experiment, the 37 prevalent shared cancer neoantigens identified by the clinico-genomic analysis described above (Table 3), as well as a subset of 11 additional antigens, were tested for binding. Regarding HLA allele coverage, of the 15 HLA alleles identified in the in silico screen, only 15 were available in the appropriate conditional HLA complex format needed for the TR-FRET assay (FIG. 1B). Consequently, the final TR-FRET assay was used to probe stable binding of neoepitopes from 47 shared cancer neoantigens across the 15 most prevalent HLA alleles (Table 4), resulting in the characterization of 24,149 neoepitope-HLA complexes.

TABLE 4
Exemplary 15 HLA Alleles

A*01:01
A*02:01
A*03:01
A*11:01
A*24:02
B*07:02
B*08:01
B*35:01
B*51:01
C*03:04
C*04:01
C*05:01
C*06:02
C*07:01
C*07:02

The TR-FRET assay utilized previously described conditional HLA ligands, peptides containing UV cleavable non-natural amino acids, to create conditional HLA complexes (HLA alpha chain, and Beta-2-microglobulin [B2M]) for the 15 HLA alleles (Darwish et al., 2021). In the TR-FRET assay, the conditional HLA complexes were incubated with the neoepitope of interest at a 100-fold molar excess and exposed to UV light for 25 min, which was expected to cleave the conditional ligand and convert the peptide from a stable high affinity “binder” to an unstable binder that dissociates from the HLA grove. In the presence of a binding neoepitope, peptide exchange occurred and stabilized the HLA complex (i.e., conditional ligand, HLA alpha chain, and Beta-2-microglobulin [B2M]; FIG. 1B, top). In the presence of a non-binding neoepitope, peptide exchange did not occur, and the HLA complex dissociated (FIG. 1B, bottom). These two different outcomes were monitored using a simple TR-FRET assay, where a TR-FRET donor (europium) was conjugated to an anti-B2M antibody and the TR-FRET acceptor was conjugated to streptavidin, which binds to the biotinylated HLA alpha chain. In these assays, a TR-FRET signal will only occur if the HLA complex remains intact due to the presence of a binding neoepitope. Samples were also heated at 37° C. for 24 hours prior to analysis to ensure identified binders formed a stable complex at physiological temperatures. TR-FRET signals were quantified based on the ratio of relative fluorescent units and signals were subjected to a double normalization to generate a robust Z score (RZ-score) for neoepitope comparison and ranking as described in the material and methods section herein. In the current analysis, any neoepitope-HLA combination with a RZ-score≥5 was considered to be a “stable binder”, while this cutoff resulted in the identification of 587 unique neoepitope-HLA pairs. This cutoff resulted in the identification of unique neoepitope-HLA pairs illustrated in Table 5. For comparing the results of the TR-FRET assay to computational prediction methods, NetMHCpan 4.0 (a.k.a., NetMHC) was employed to predict neoepitope presentation of the 24, 149 neoepitope-HLA pairs assayed by TR-FRET. For this analysis the “eluted ligand” percentile rank (% Rank) values were used to determine if a neoepitope was a “binder” (e.g., % Rank≤2), resulting in identification of 408 unique predicted neoepitope-HLA pairs.

TABLE 5
Targeted assays contained mixture of peptides that demonstrated
in vitro binding & peptides predicted to bind

| | # Binders | # Peptides |
|---|---|---|
| Allele | (Robust Zscore >=5) | Targeted |
| A*01:01 | 76 | 88 |
| A*02:01 | 45 | 52 |
| A*03:01 | 61 | 82 |
| A*11:01 | 42 | 57 |
| A*24:02 | 40 | 47 |
| B*07:02 | 57 | 70 |
| B*08:01 | 55 | 79 |
| B*35:01 | 40 | 46 |
| B*51:01 | 19 | 21 |
| C*07:01 | 13 | 36 |
| C*07:02 | 9 | 28 |
| C*04:01 | 28 | 38 |
| C*06:02 | 34 | 54 |
| C*05:01 | 34 | 53 |
| C*03:04 | 34 | 49 |
| | SUM = 587 | SUM = 800 |

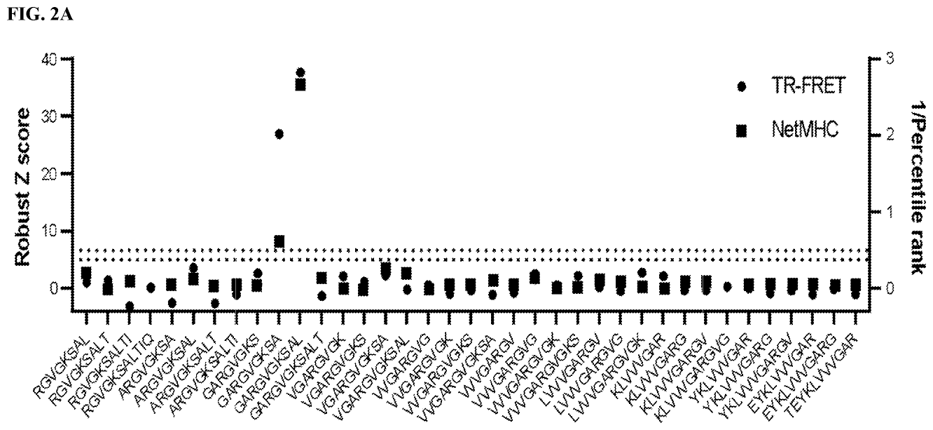

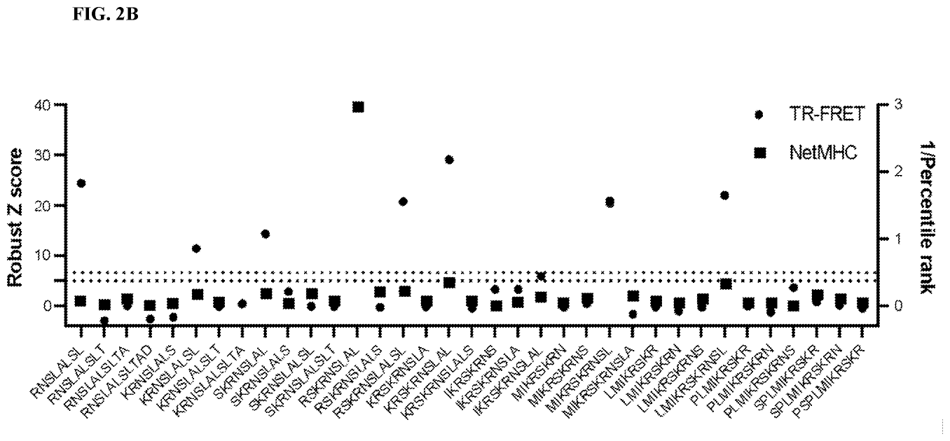

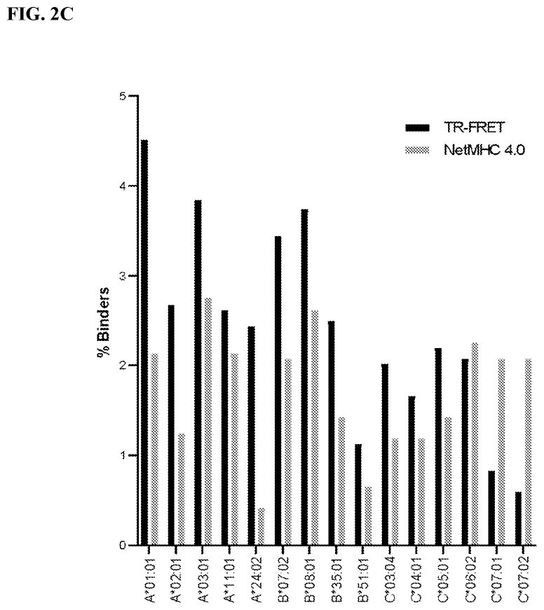

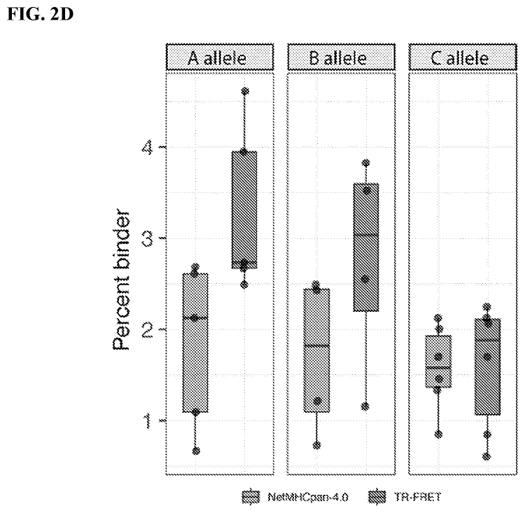

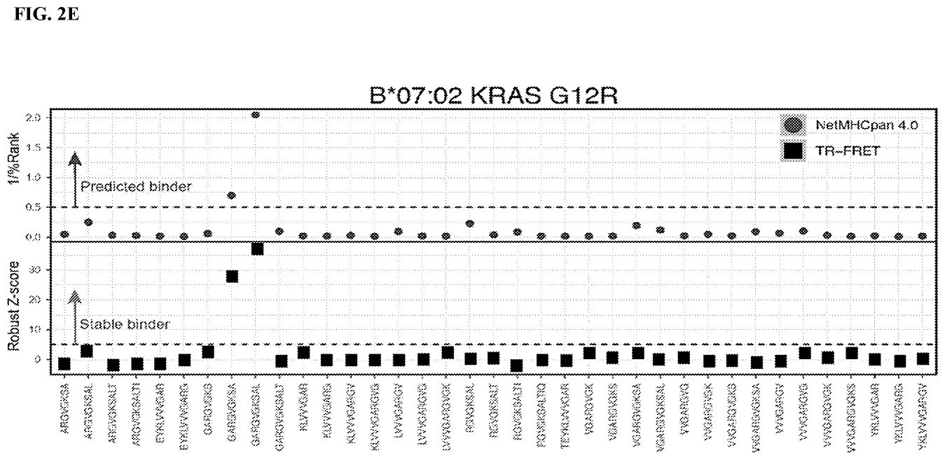

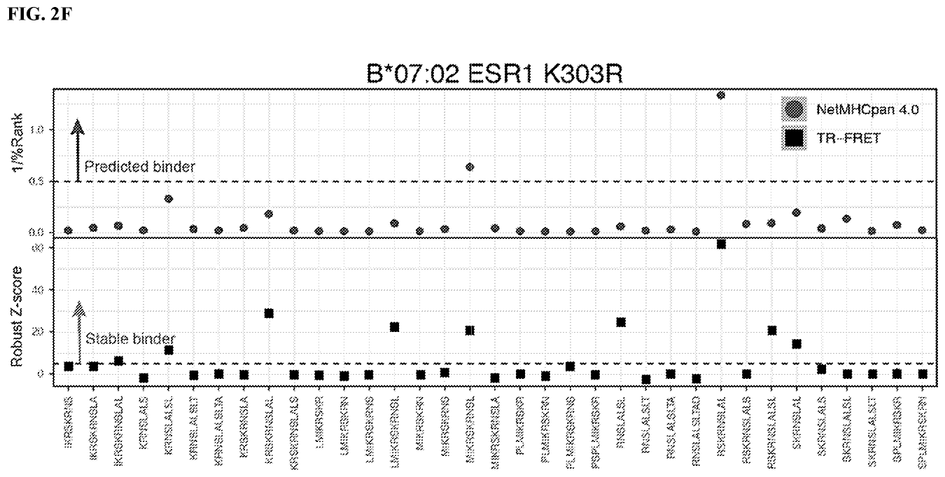

A comparison of the TR-FRET Robust Z-score and inverse percentile rank scores are shown in FIGS. 2A and 2E. Robust Z-scores with values greater than 5 were considered binders (above black dotted line) and as shown in FIG. 2A, there was a strong correlation between the NetMHC 4 binding prediction (above the dotted lines) and the TR-FRET analysis where both identified the same two KRAS G12R neoepitopes as stable binders to B*07:02 (FIG. 2E). This correlation was not consistently observed across all neoantigen-allele combinations. Because TR-FRET generally identified more stable neoepitope-HLA pairs, there were several cases where NetMHC did not predict the same binding events identified by TR-FRET. For example, in the comparative analysis for ESR K303R neoepitopes across the B*07:02 allele, the TR-FRET assay identified 9 stable binding neoepitopes and NetMHC 4.0 only predicted 2 of these binders (FIGS. 2B and 2F). When the criteria described above (Robust Z-score>5 and Percentile rank<2) were used to distinguish binders and nonbinders neoepitopes, the % binders identified by TR-FRET and NetMHCpan 4.0 was 2.45% and 1.72%, respectively, of all neoepitope-HLA combinations tested. FIG. 2C shows the distribution of % binders across the different alleles for both the TR-FRET and NetMHC 4.0 analysis and on average NetMHC 4.0 yielded a lower percentage of binder compared to the TR-FRET analysis. FIG. 2D also shows that, when measured as a percentage of all potential neoepitope-HLA complexes, TR-FRET generally identified more stable binders as compared to NetMHC, particularly for HLA-A and HLA-B alleles.

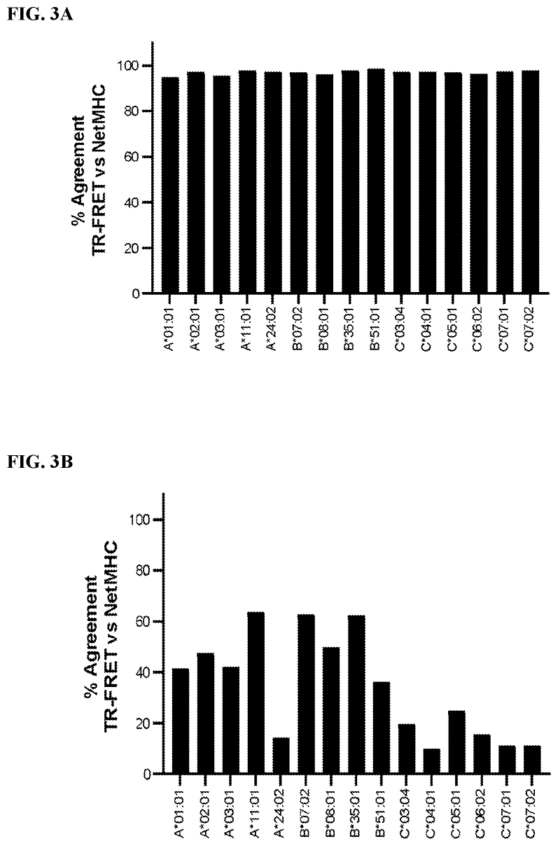

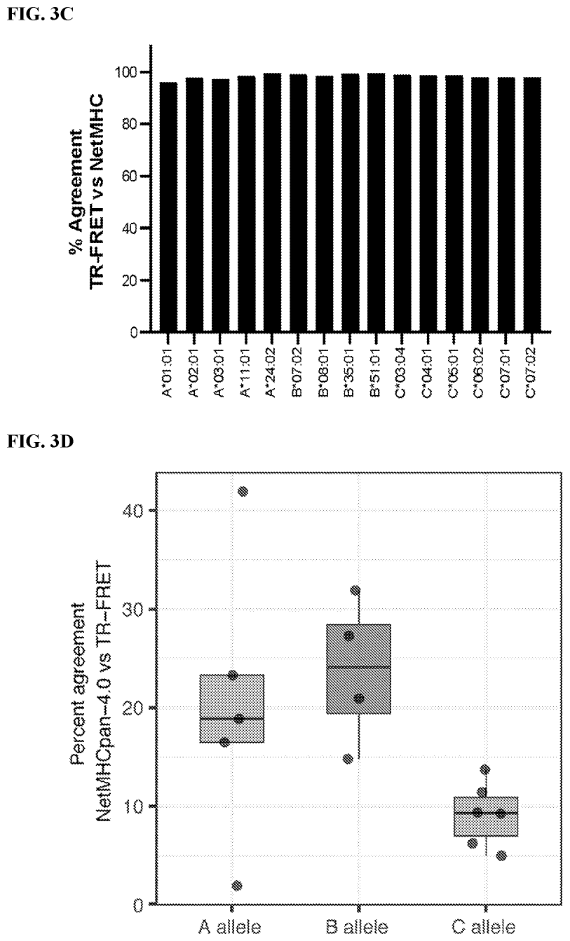

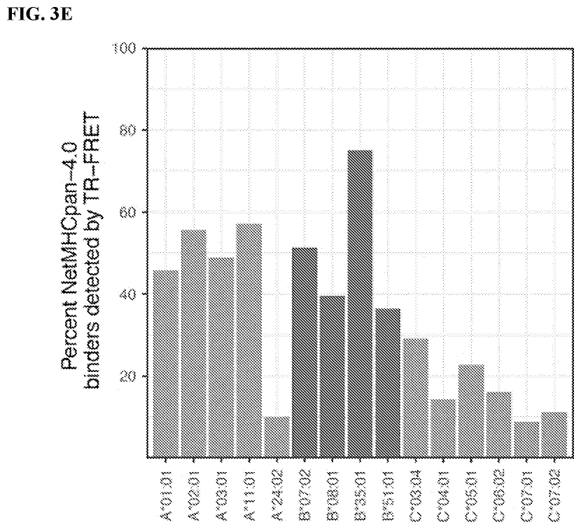

To better understand an overall correlation between the NetMHC 4.0 prediction and TR-FRET measurements, the % of agreement in classifying binders and non-binders across the two methods was compared (FIG. 3A). The % agreement between these methods across all the alleles was very strong and ranged between 95.1-98.6%, depending on the allele (FIG. 3A). To further assess the correlation between these two methods, the overlap was evaluated for the neoepitopes classified as binders by the two methods. In contrast to when all neoepitopes were compared (binder+non-binders), if only the identification of binders was considered, the overlap between the two methods dropped significantly (FIG. 3E) and the top performing alleles were around 40-60% agreement between the two methods and some alleles were as low as 10% (see, e.g., the agreement of mere 2.04% to 40% in FIG. 3D). Agreement was generally higher for HLA-A and HLA-B alleles as compared to HLA-C alleles (FIG. 3D). However, A*24:02 exhibited the lowest agreement at 2.04% (FIG. 3E). When this same analysis was performed only considering the non-binders, there was very strong agreement ranging from 94.1 −98.4% (FIG. 3C). These results demonstrate that both methods generally agree on non-binding events, whereas positive interactions are found with minimal overlap. This substantiates the power of combining both approaches to identify and prioritize unique neoepitope-HLA pairs for further characterization. These results were not surprising because the vast majority of the neoepitopes were classified as non-binders.

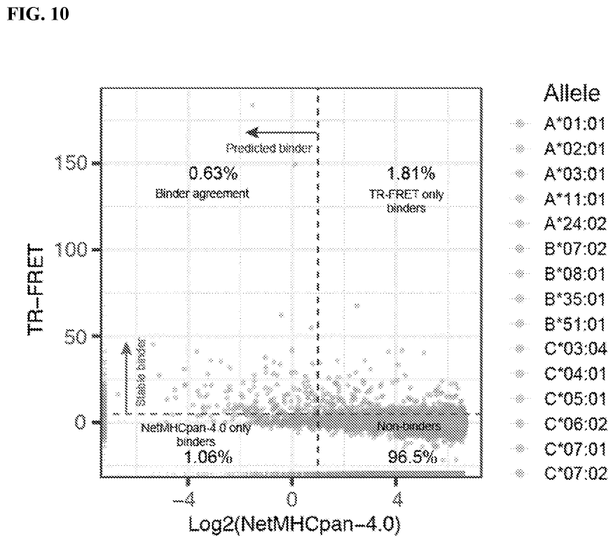

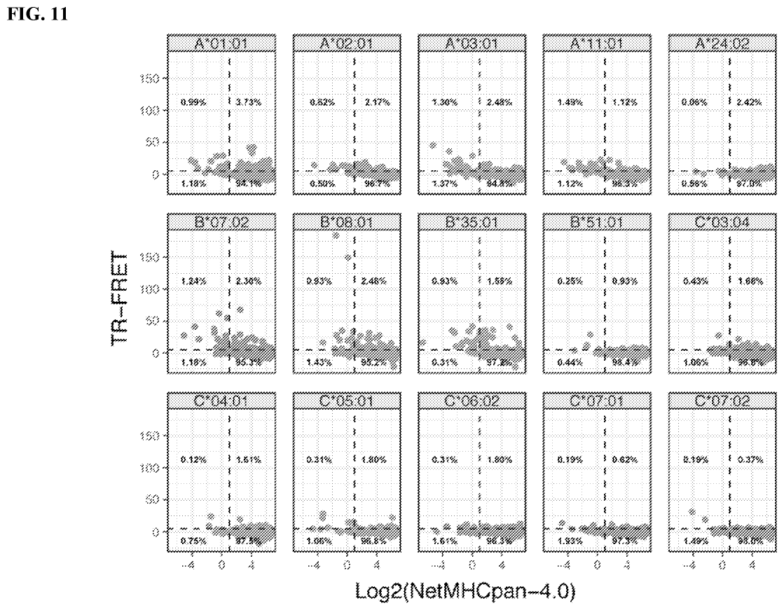

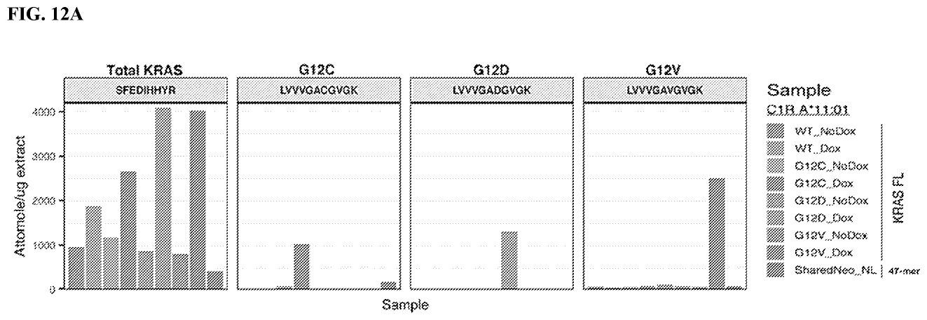

To better visualize the complementarity of binder identification by TR-FRET and NetMHC, the TR-FRET RZ-score and NetMHC % Rank were plotted for all candidate neoepitope-HLA pairs (FIG. 10). About 0.63% of all candidate neoepitope-HLA pairs were found to be binders by both methods. When data were viewed at the allele level, agreements varied from 0.06% to 1.49% of all potential neoepitope-HLA pairs (FIGS. 10 and 11). Interestingly, each method identified nearly the same percentage of additional binding events for neoepitope-HLA pairs, 1.06% for NetMHC and 1.81% for TR-FRET, demonstrating that each method has the potential to identify unique binding combinations (FIG. 10).

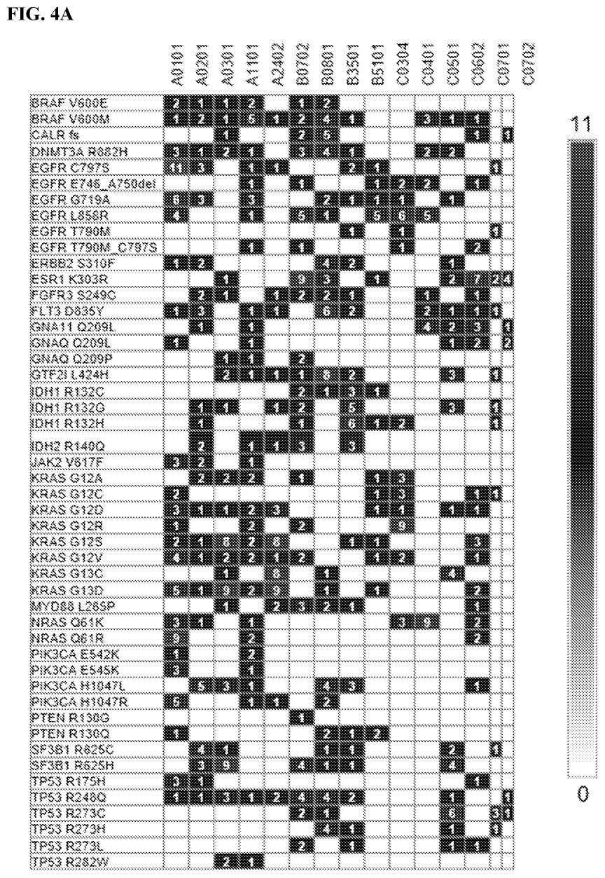

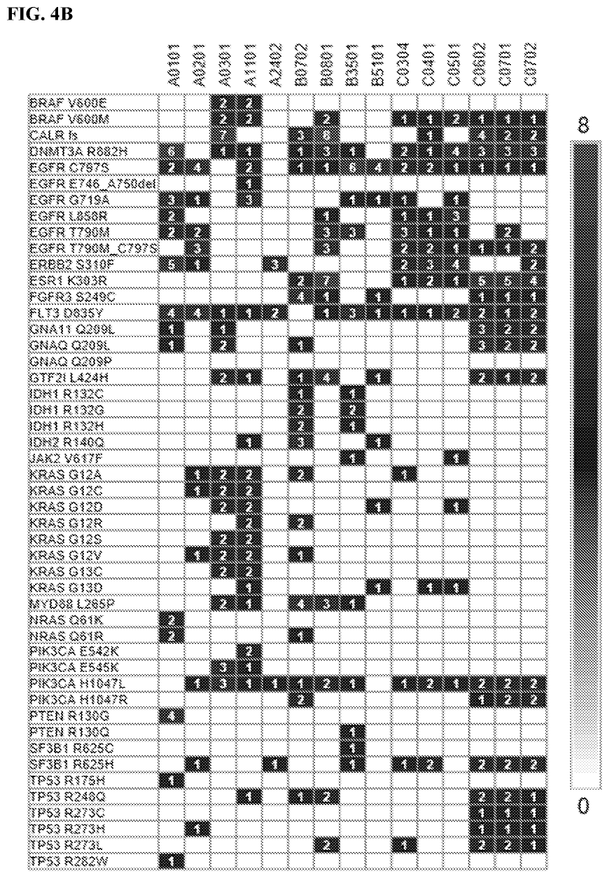

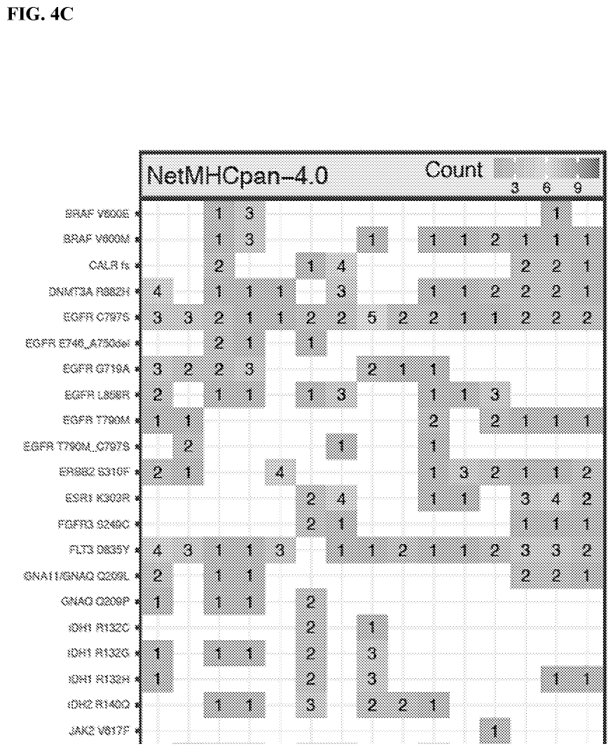

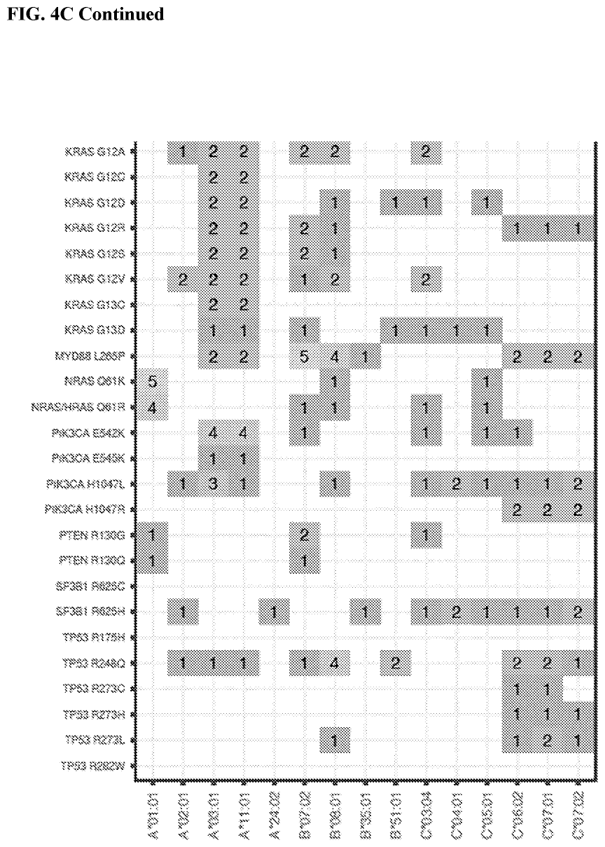

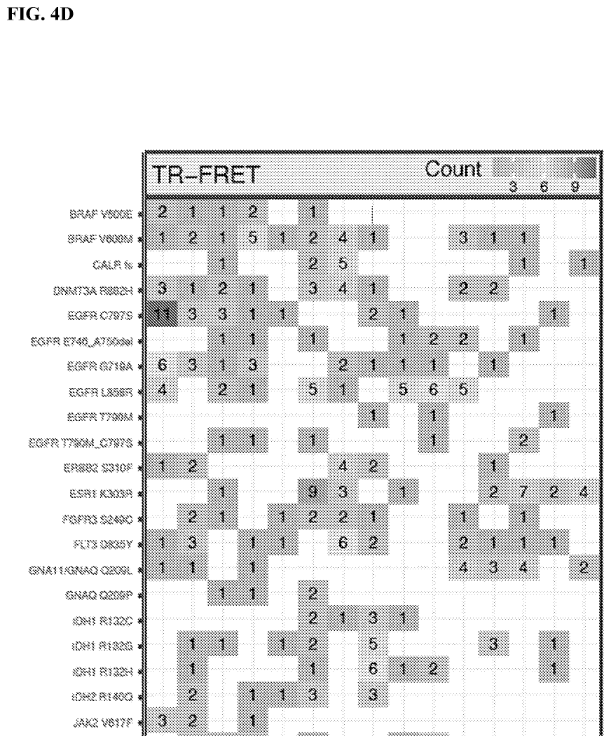

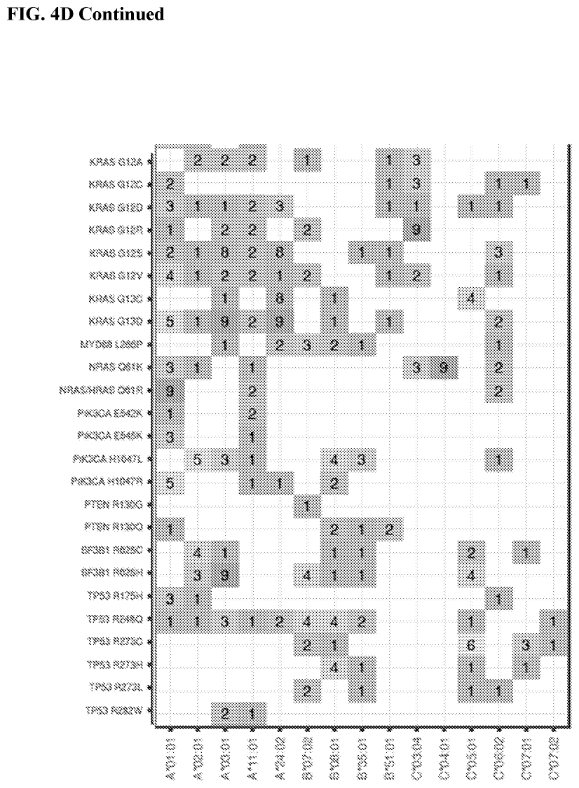

To more clearly visualize discrepant binders observed by the TR-FRET and NetMHC analysis, a heatmap was generated to display the number of binders for each HLA allele and neoantigen combination for the TR-FRET (FIG. 4A) and NetMHC (FIG. 4B) analysis. A similar experiment testing 47 candidate neoantigens (i.e., not including the GTF2I L424H neoantigen) produces a similar results (FIGS. 4C and 4D). Based on this analysis there are clear areas of overlap as well as significant gaps between the different analyses. For example, TR-FRET identified 11 epitopes as binders for EGFR C797S and A*01:01, whereas NetMHC only predicted 3 binding epitopes (FIGS. 4C and 4D). Interestingly these combined results suggest that both assays can reliably distinguish non binders with a high correlation but this drops significantly when the analysis is performed on binders. These findings also provide further evidence of the value added by including a biochemical binding screen in addition to the prediction algorithm when selecting neoepitopes to include in the targeted mass spec analysis to measure neoepitope presentation. These findings highlight the power of the high-throughput biochemical assay to identify a potentially complementary set of neoepitope-HLA pairs and suggest using both methods together could lead to more comprehensive neoepitope discovery.

### Cell Engineering—Generation of HLA-I Monoallelic Cell Lines Co-Expressing 47 Shared Cancer Neoantigens

Despite observed peptide-HLA stabilization in vitro, expression and processing of a mutated protein may not result in presentation of a neoepitope in a cellular context (Jappe et al., 2018 Immunology 154(3):407-417; Garstka et al., 2015 Proc Natl Acad Sci USA. 112(5): 1505-1510). For this reason, validation of candidate neoepitopes typically requires genetic expression of target neoantigens followed by a readout of association with surface-bound HLA. The process of neoantigen-HLA discovery has been enhanced through the use of engineered “HLA monoallelic” cell lines, although these have relied to a large degree on endogenous mutant protein expression or expression of relatively few mutant transgenes, thus limiting throughput (Wang et al., Direct Detection and Quantification of Neoantigens. Cancer Immunol Res. 2019; 7(11): 1748-1754; Bear et al., 2021 Nat Commun 12(1):4365; Abelin et al., Mass Spectrometry Profiling of HLA-Associated Peptidomes in Mono-allelic Cells Enables More Accurate Epitope Prediction. Immunity 2017; 46(2):315-326).

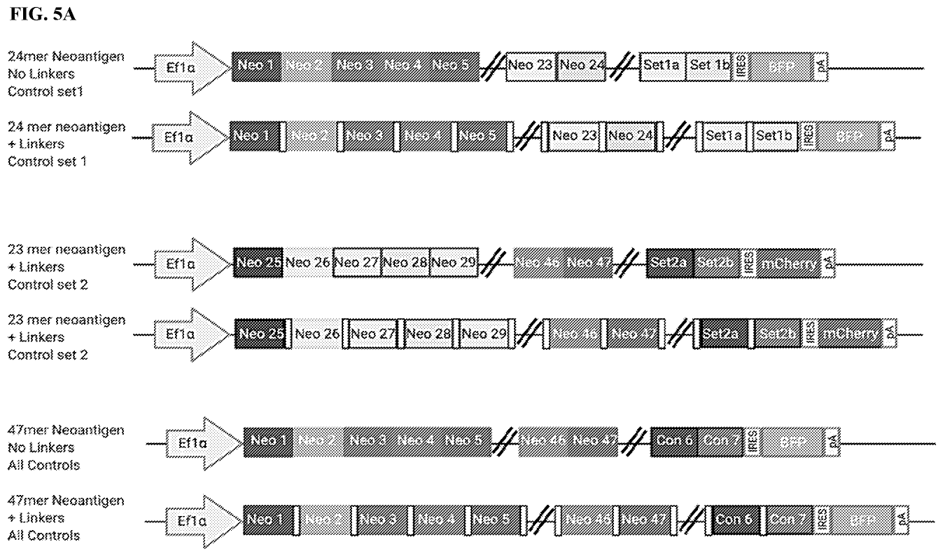

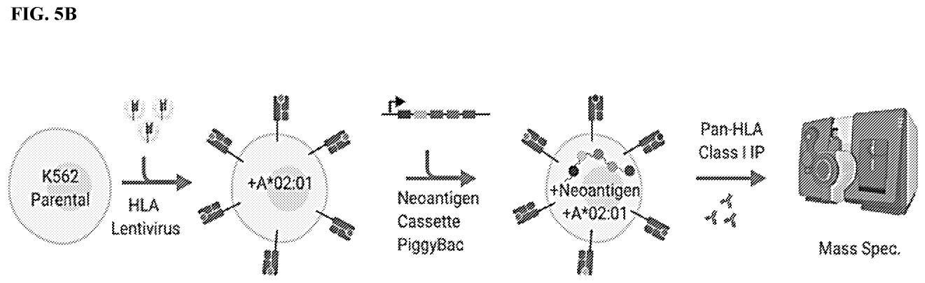

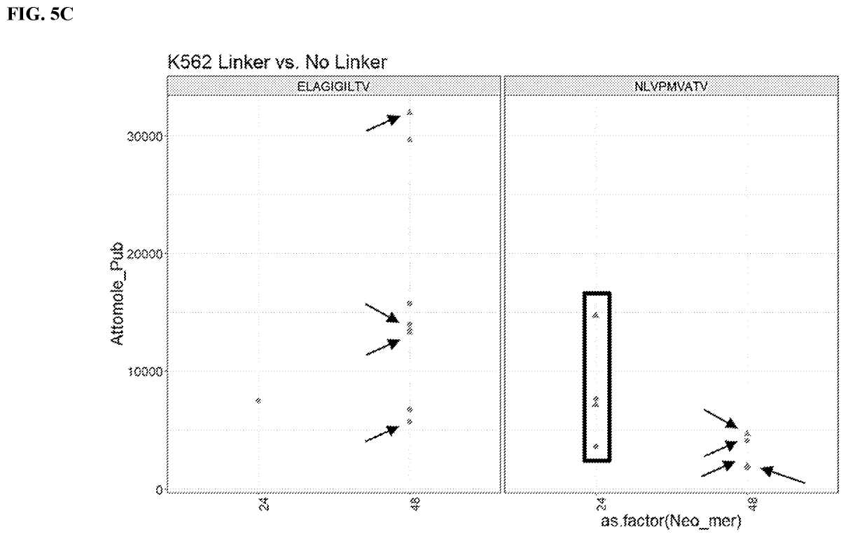

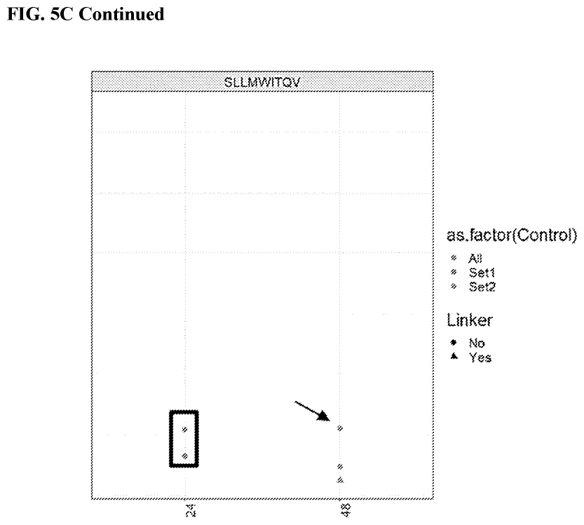

Following the detection of peptide-HLA complexes via the high throughput TR-FRET binding assay, these identified epitopes were validated in a cellular context. Simultaneous encoding of all 47 candidate neoantigens within a single HLA-null cell line would dramatically improve throughput of cell line generation and subsequent validation of TR-FRET identified neoepitope-HLA pairs by targeted mass spectrometry. Because no cell line expresses all 47 neoantigens, a piggybac cassette was generated to contain a concatenated neoantigen expression array (FIG. 5A). As local sequence context may potentially affect neoantigen processing, vectors were used where the neoantigens were concatenated (no-linker) or separated by g/s enriched linker sequences (linkers). Several viral peptides known to be presented by A*02:01 at the C-terminus were used as control, to confirm that the entire polypeptide sequence was efficiently expressed.

In order to validate the expression neoantigen strategy, a series of Piggybac neoantigen expression constructs were introduced into a K562 cell line stably expressing an A*02:01 allele (FIGS. 5A and 5B). HLA IP+LC-MS were used to detect presentation of the expected viral control peptides (FIG. 5C). While some variability was observed between the linker/no linker cassettes at the neoantigen level, no obvious trend emerged to indicate which expression strategy was superior. In addition, there was no significant difference in control peptide detection between lines expressing 23, 24 or 47 neoantigens. Based on these data the 47-mer neoantigen constructs were chosen for further experimentation in both the linker and no-linker sequence context.

The K562 cell line has been used historically as a model cell line for HLA-antigen presentation studies due to the low level of endogenous HLA expression. While relatively low, however, this line expresses detectable levels of HLA-C*05:01 and C*03:04. Furthermore, HLA expression can be upregulated in response to certain stimuli. Preliminary studies revealed the presence of putative HLA-C*05:01 peptides in the HLA-A*02:01 ‘monoallelic’ cell lines, indicating that K562s may not be an ideal model system for the assay.

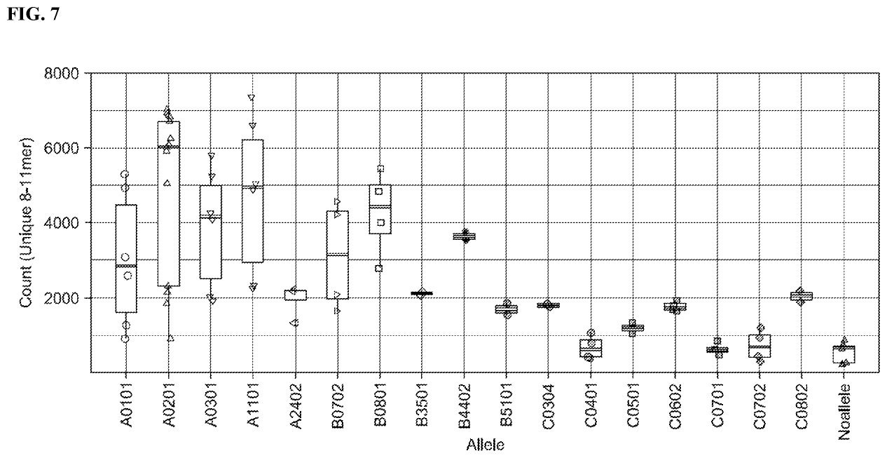

In another exemplary experiment, the HMy2.C1R lymphoblast cell line (a.k.a., C1R) was chosen as the model system since it is a robust, fast-growing line that has little to no expression of its endogenous HLA-A or B protein but maintains the ease of handling and robust expression of suspension cells in culture (FIG. 6B) (for the HLA-C*04:01 allele, see Creary et al., 2019 Hum Immunol 80(7):449-460). Using a CRISPR/Cas9 knockout strategy, the remaining HLA-C allele (HLA-C*04:01) was disrupted from the HMy2.C1R cell (FIG. 6A), generating an HLA-Class I knockout (Class I KO) HMy2.C1R population (FIGS. 6B and 6C). The resulting HLA-null (C1R^{HLAnull}) population was enriched by cell surface stain using a pan-HLA-I antibody and fluorescence activated cell sorting (FACS). There is precedence in the literature suggesting expression of B*35:03 in the C1R cell line (Schittenhelm et al., A comprehensive analysis of constitutive naturally processed and presented HLA-C*04:01 (Cw4)-specific peptides. Tissue Antigens. 2014; 83(3): 174-9). However, in the instant studies no evidence was observed for the B*35:03 motif in the immunopeptidomic analysis of the resulting C1R^{HLAnull} cells. To generate desired panel of neoantigen expressing monoallelic cell lines, Class I KO populations were first generated to stably express the linker or no linker Piggybac neoantigen cassette. 17 HLA variants of interest were lentivirally transduced into these parental cells to generate 34 total populations for further analysis (FIG. 6D). A summary of 8-11-mer unique peptides per allele for the 17 HLA variants of interest is shown in FIG. 7.

Following development of a C1R^{HLAnull} cell population, synthesis and delivery of piggyBac expression vectors enabled stable transgene integration. In an exemplary experiment, two foundational cell lines, as in FIG. 6E, were generated, each co-expressing all 47 prioritized neoantigens in distinct configurations, to test whether local sequence context has the potential to affect antigen processing (Gomez-Perosanz et al., Identification of CD8″ T cell epitopes through proteasome cleavage site predictions. BMC Bioinformatics. 2020; 21(Suppl 17):484). These two cell lines differed by the presence (“Linker”) or absence (“No-Linker”) of short amino acid linker sequences between most neoantigen segments within the polyantigen cassette; hereafter these cell lines are referred to as linker and no-linker, respectively. Stable linker and no-linker cell populations were enriched using a separate TagBFP2 (BFP) marker to select for transgene-positive C1R^{HLAnull} cells. HLA-I monoallelic cells were then created by the introduction of the 15 HLA alleles (Table 4) as individual transgenes through stable lentiviral transduction of the linker and non-linker neoantigen-expressing C1R^{HLAnull} cell lines, resulting in 30 total cell populations for further analysis. HLA expression was confirmed by cell surface stain using a pan-HLA antibody (FIGS. 6F and 6G). For validation of the functionality of these polyantigen cassettes, the linker and no-linker neoantigen constructs contained a set of control antigens with epitopes known to be presented by of A*02:01. HLA immunopeptidomics confirmed the presence of these peptides in both the linker and no linker HLA-A*02:01-engineered cells (FIG. 6H).

In addition to HMy2.C1R cell, a similar result was detected by experiments using K562 cells.

### Engineered Polyantigen Cassettes Augment Neoepitope Presentation

One potential concern with the approach described above was that epitopes derived from a construct containing 47 concatenated neoantigens may not reflect epitopes derived from a full-length mutant protein. This was further investigated in the context of KRAS due to the recent description of A*11:01 restricted 9-mer and 10-mer neoepitopes detected by a targeted proteomic assay (Bear et al., 2021 Nat Commun 12(1):4365). To this end, three C1R^{HLAnull} cell lines were developed to express HLA-A*11:01 as well as a doxycycline (dox)-inducible full-length, wildtype, G12C, G12D, or G12V mutant KRAS protein. The neoepitope presentation from these cell lines was then compared with the presentation from a cell line expressing the no-linker variant of the polyantigen cassette.

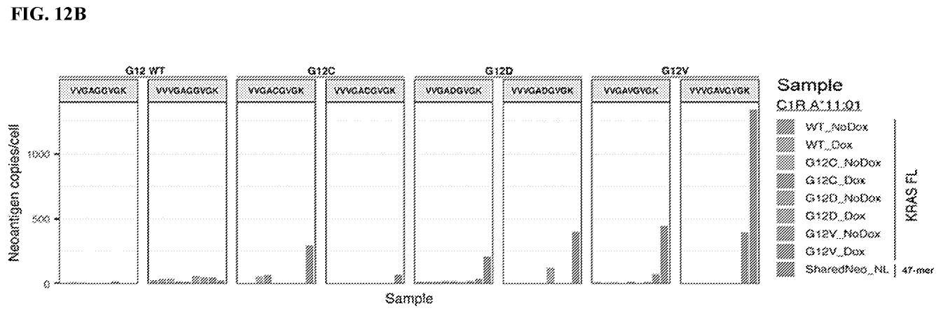

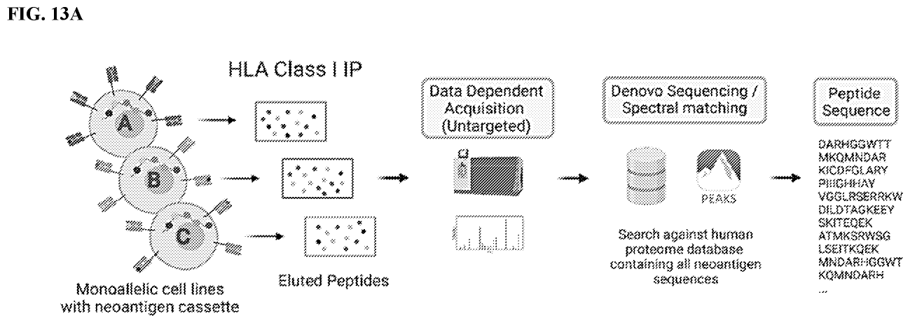

Mutant protein expression was confirmed by a whole-cell targeted proteomic assay comprising a peptide that can detect total KRAS as well as three unique peptides that measured individual KRAS mutants (FIG. 12A). This analysis validated dox-induced over-expression of the KRAS alleles by demonstrating an increase in total KRAS detected when dox was added to the culture medium (FIG. 12A). Note, little to no signal was found at the steady-state protein level for these mutant peptides within the cell line containing a polyantigen cassette.

A targeted immunopeptidomic assay was then used to quantify the level of presentation of previously identified 9-mer and 10-mer KRAS epitopes within the same cell lines described above (FIG. 12B). For the cell lines containing a full length mutant protein, induction of neoepitope presentation was observed for both G12V epitopes as well as the 10-mer epitope of G12D (FIG. 12B). A weak signal was detected for the 9-mer epitope of G12C in both the control and dox-treated cell line. This could have been due to leaky/basal promoter activity as a weak signal for G12C was also detected at the protein level in both conditions (FIGS. 12A and 12B). Interestingly, the 9-mer and 10-mer epitopes of all KRAS mutants were detected in the cell line expressing the polyantigen cassette. Further, the polyantigen-modified cell line also presented higher absolute copies per cell of KRAS mutant epitopes as compared to cells expressing full length protein (FIG. 12B). When combined with the lack of detection of mutant KRAS peptides at the protein level (FIG. 12A), these results suggested the protein product of the polyantigen cassette is likely unstable and efficiently degraded such that epitope presentation is enhanced. This effect has been demonstrated previously in systems where induced protein degradation was used to increase presentation of epitopes derived from the degraded protein (Moser et al., 2018 Front Immunol. 8:1920; Jensen et al. 2018 Front Immunol. 9:2697). Therefore, monoallelic cells containing the polyantigen cassette provided both a higher-throughput and more sensitive system for discovery of neoepitopes from shared cancer neoantigens.

### Detection of Neoepitope Presentation on Cell Lines Engineered to Present Shared Neoantigens

In one exemplary experiment, after the generation of monoallelic cell lines expressing a polyantigen cassette with or without linkers, peptide presentation was validated by HLA immunoprecipitation followed by both untargeted and targeted mass spectrometry (MS) analysis. Untargeted MS analysis enabled unbiased identification of peptides from the entire immunopeptidome, including peptides derived from our neoantigen constructs, but is limited in the sensitivity of detection. Targeted analysis enabled sensitive detection of peptides presented at low copies per cell, but was constrained to peptides identified as binders within the TR-FRET assay as well as select peptides predicted to bind by NetMHC.

### Untargeted MS

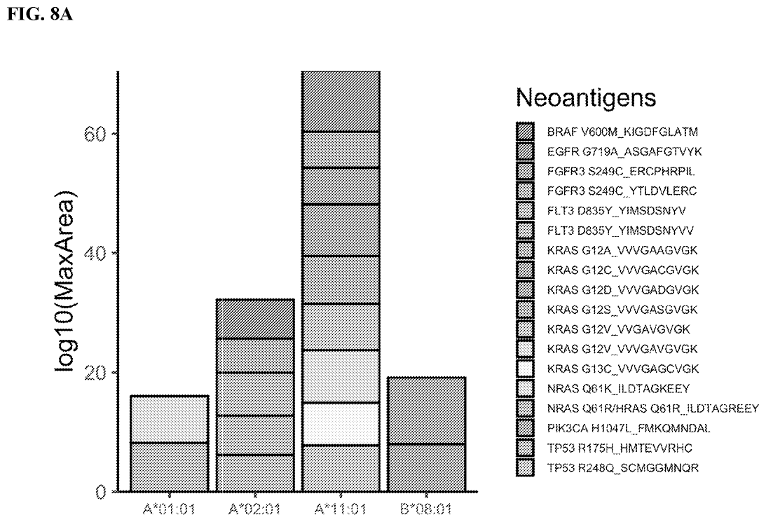

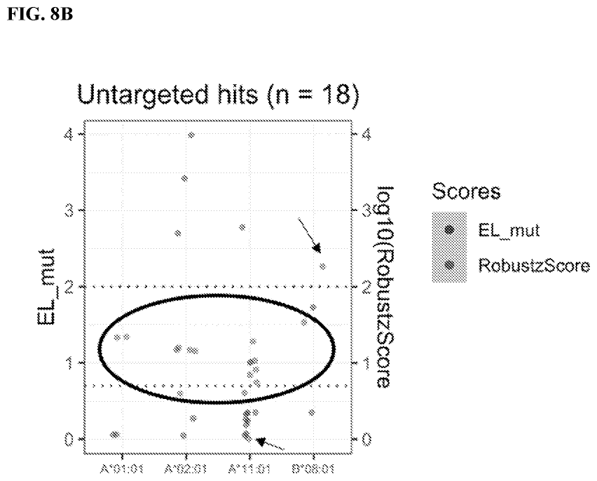

Untargeted MS data identified 852 to 7342 unique 8-11-mer peptides across all samples, with the largest number of peptides identified in HLA-A alleles and the smallest numbers of peptides identified in HLA-C alleles (FIG. 7). For each allele sequence motifs were generated to demonstrate that the presented peptides fit the expected motifs as derived from previous publications. Within the untargeted analysis 18 neoepitope-HLA pairs were found from 15 shared neoantigens across 4 HLA alleles (FIG. 8A) representing ˜3.455% of neoepitope-HLA pairs predicted by NetMHC and ˜3.005% of neoepitope-HLA pairs identified within the TR-FRET assay. Interestingly, ˜77.8% of these peptides exhibited NetMHC presentation prediction scores≤2 and 83.3% of these binders had a measured Robust ZScore≥5 (FIG. 8B).

In addition to neoantigen derived epitope peptides, untargeted analysis enabled the detection of peptides derived from the non-mutation containing portions of each neoantigen 27-mer region, as well as junction regions of the 49-mer neoantigen construct that connect sequential 27-mers. We found junction peptides from the non-linker and linker constructs. The lower number of peptides from the linker construct may probably be due to the fact that the linker comprised G and S amino acids, which are not typically anchor residues. Lastly, viral control peptides as well as peptides stemming from BFP were also identified, indicating that the neoantigen construct was expressed in each of the tested cell lines.

### Targeted MS

While untargeted analysis enables identification of thousands of peptides, it is challenged by stochastic sampling of peptides for identification and the requirement of detecting an intact peptide species within a survey scan which limits detection of peptides that are presented at a low level. Conversely, targeted MS analysis dedicates the entirety of the instrument duty cycle to the analysis of a small number of peptides (e.g. ˜ 100), improving data reproducibility and detection of peptides presented at low copies per cell. Due to the cost of synthesizing heavy amino acid labeled standard peptides, a challenge of targeted proteomics is the determination of peptides to synthesize for MS analysis. Within the monoallelic system described herein, synthesizing all possible 8-11-mer peptides for our 48 neoantigens would require synthesis of ˜1,800 peptides. To limit the number of peptides for analysis, only prioritized peptides were synthesized, including those exhibiting a Robust Zscore >5 within the TR-FRET assay described above. As a control, additional peptides were synthesized from a subset of mutations that had a predicted NetMHC presentation score<2%, but were not found as binders in the TR-FRET assay. Taken together, peptides were analyzed across 17 alleles with each individual assay comprising peptides analyzed by targeted analysis.

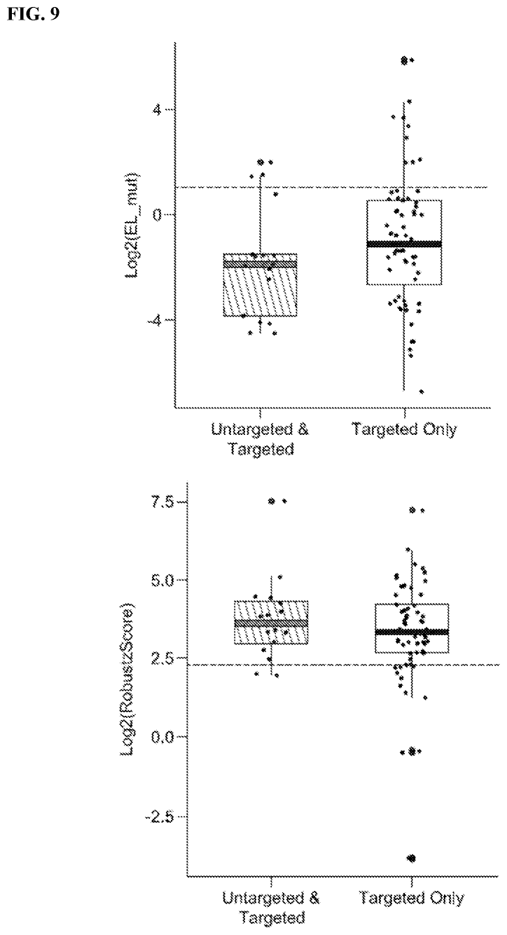

Following targeted analysis 81 neoepitope-HLA pairs were identified. Of these, all but one of the epitopes (BRAF Epitope) was also identified in untargeted analysis. Interestingly, neoepitope-HLA pairs identified by targeted analysis only had an increased presentation score by NetMHC and a slightly lower Robust ZScore in our TR-FRET analysis (FIG. 9). One advantage of performing targeted analysis is the ability to calculate absolute levels of peptides, demonstrating that the median level of epitope presentation spanned from ˜50 amol to 200 fmol. Importantly, the absolute quantification can be compared across independent replicates of immunopeptidome analysis (i.e., cell culture, MHC-IP, and MS analysis) and comparison of the replicates demonstrated general agreement of absolute amount of peptide presented. As with control peptide analysis, no clear trends linking presentation level and presence of a linker within the neoantigen construct.

Absolute quantification within targeted proteomics enabled by the inclusion of synthetic peptides that include isotopically heavy amino acids, however, due to the cost of such reagents, synthesis of all possible HLA peptides from the 48 shared neoantigens was not practical.

In another exemplary experiment, following the generation of 15 monoallelic cell lines harboring 47-mer shared neoantigen constructs with or without linkers, as described above, peptide presentation was validated by mass spectrometry using both untargeted and targeted mass spectrometry (MS). Untargeted MS analysis enabled unbiased identification of peptides from the entire immunopeptidome, including peptides derived from the neoantigen constructs, but was limited in the sensitivity of neoepitope detection. Targeted analysis enabled sensitive detection of peptides presented at low copies per cell, but was constrained to peptides identified as binders within the TR-FRET assay as well as select peptides predicted to bind by NetMHCpan-4.0.

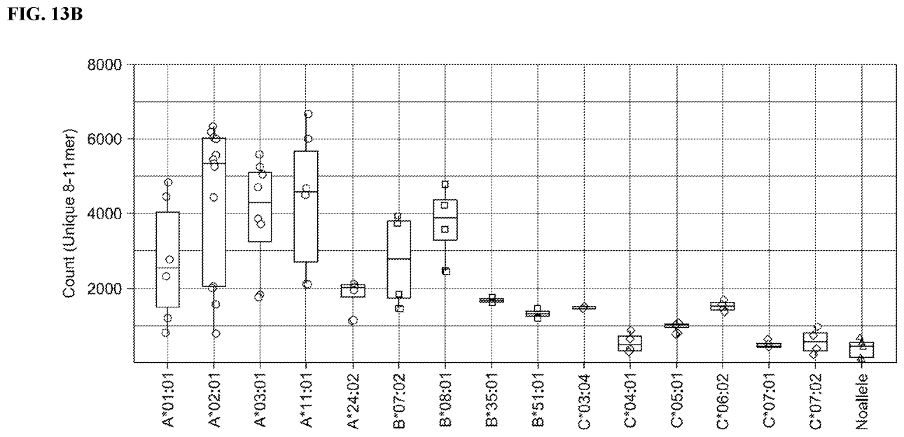

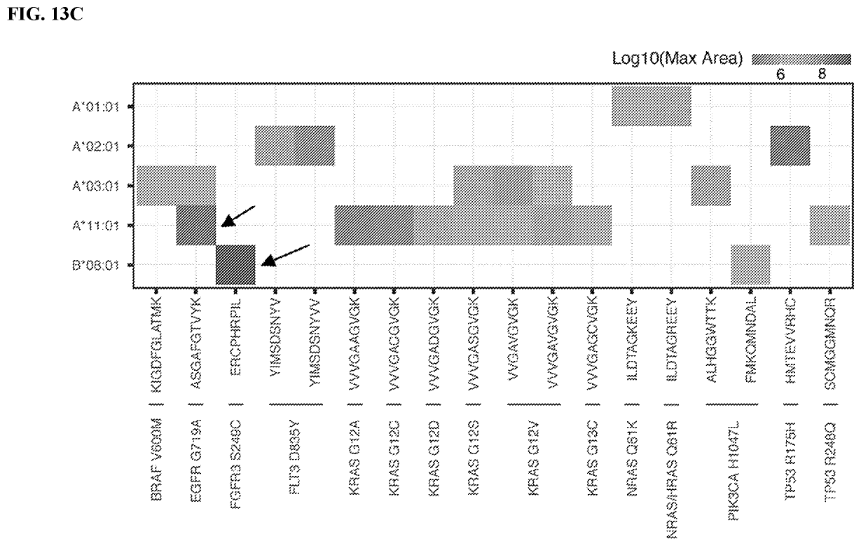

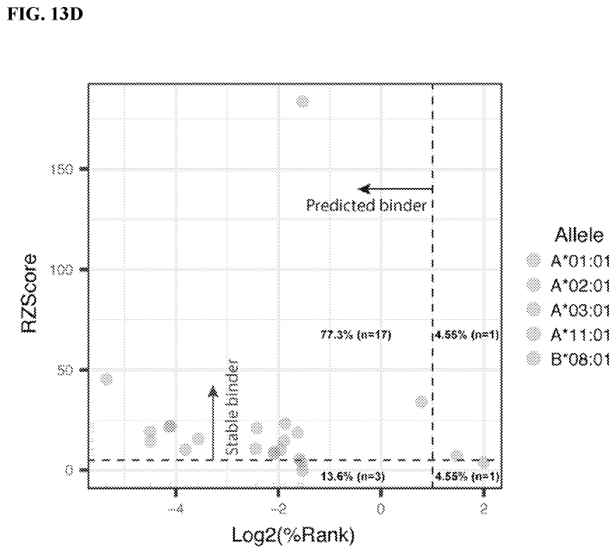

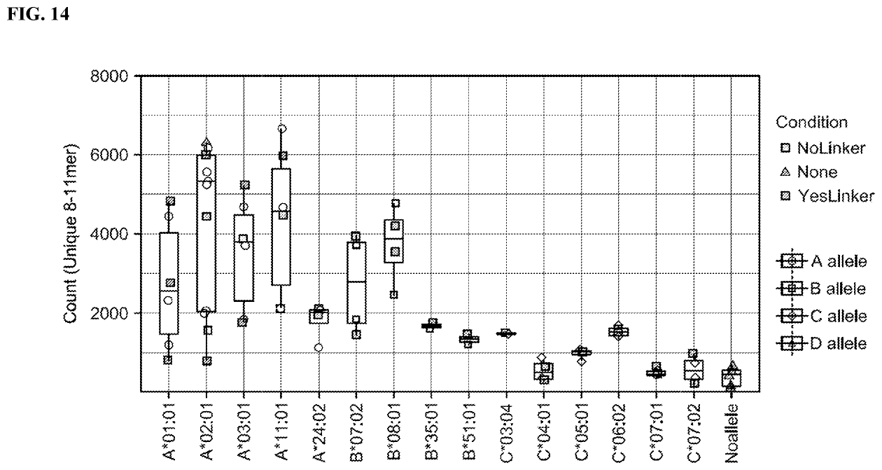

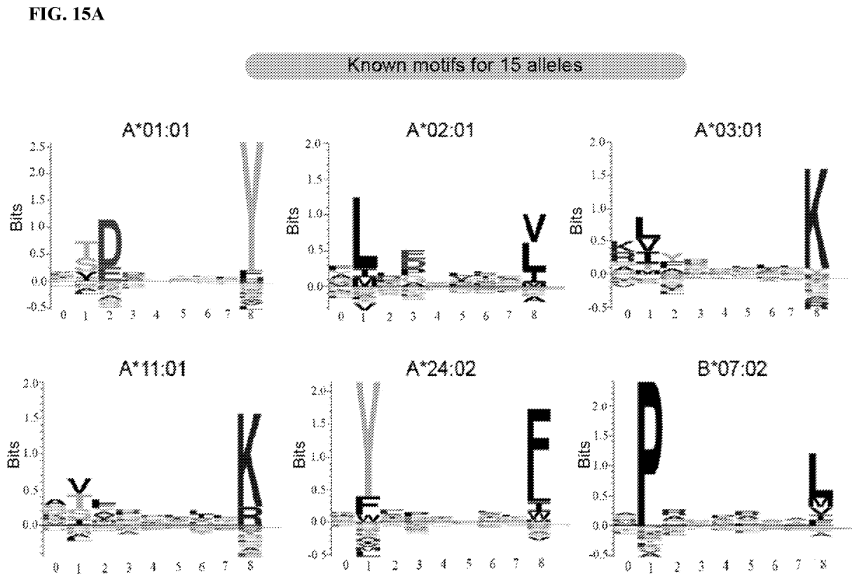

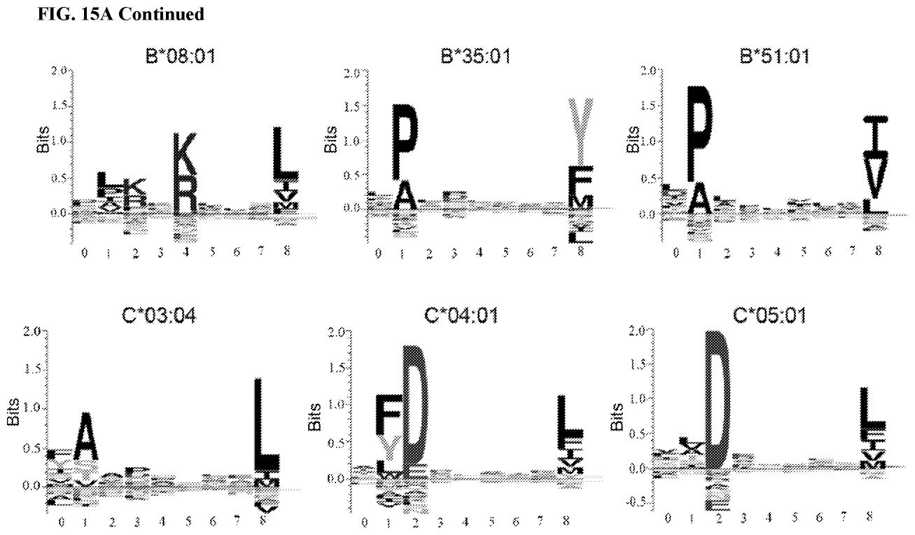

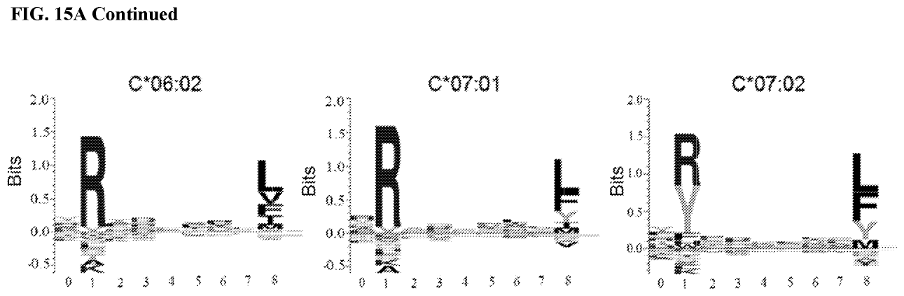

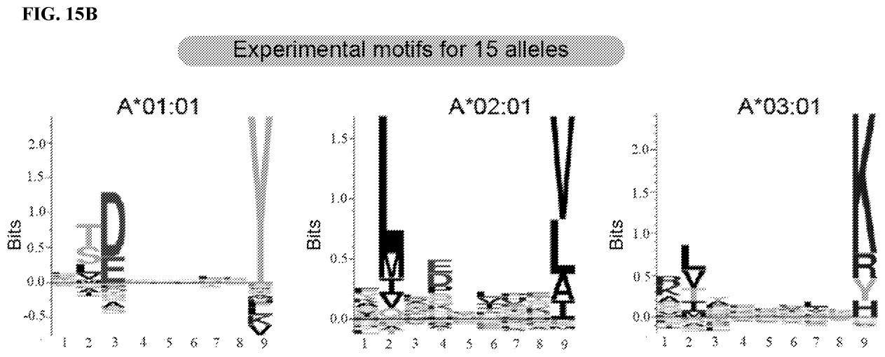

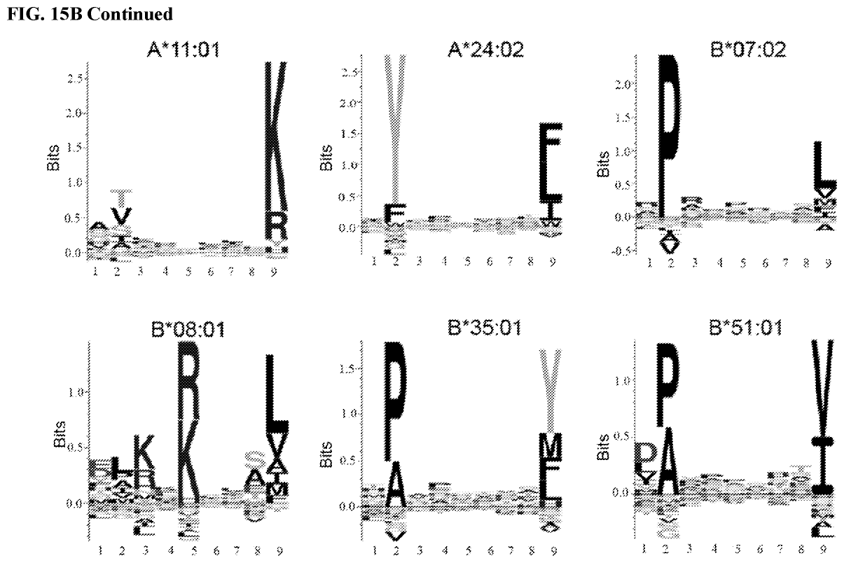

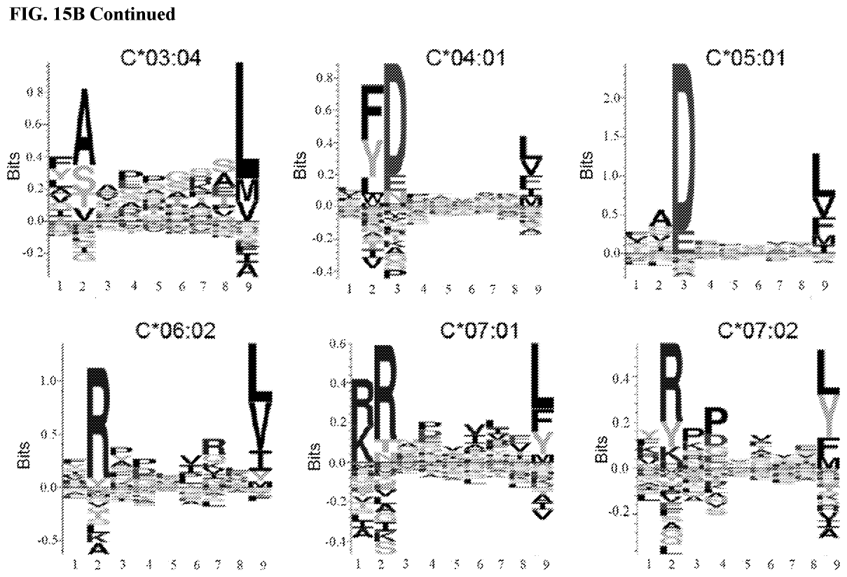

For untargeted MS analysis of each monoallelic cell line, the resulting data were searched using PEAKS (Zhang et al., PEAKS DB: de novo sequencing assisted database search for sensitive and accurate peptide identification. Mol Cell Proteomics. 2012; 11(4): M111.010587) against a human proteome database appended with the linker and no-linker polyantigen cassette construct and BFP sequences (FIG. 13A). From this analysis 218 to 6,663 unique 8-11-mer peptides were identified within each monoallelic cell line, with the largest number of peptides identified in HLA-A alleles and the smallest numbers of peptides identified in HLA-C alleles (FIG. 13B). Furthermore, the number of 8-11-mer peptides and general sequence features for each allele overlapped regardless of the polyantigen linker status and confirmed that presented peptides fit expected motifs (FIGS. 14 and 15A-15B).

Through untargeted analysis 22 neoepitope-HLA pairs (represented by each of color squares in FIG. 13C; about 1% false discovery rate (FDR)) were found from 15 shared neoantigens across 5 HLA alleles representing ˜5.4% of neoepitope-HLA pairs predicted by NetMHC and ˜3.7% of neoepitope-HLA pairs identified within the TR-FRET assay (FIG. 13C). For untargeted analysis intensity measurements cannot be directly converted to absolute abundance, but neoepitopes from EGFR G719A (ASGAFGTVYK; SEQ ID NO: 113) and FGFR3 S249C (ERCPHRPIL; SEQ ID NO: 114) demonstrate much larger intensities as compared to other detected neoepitopes. For these 22 neoepitope-HLA pairs TR-FRET and NetMHC showed excellent concordance as 17 were identified as binders by both approaches (FIG. 13D). However, there were also 1 and 3 neoepitope-HLA pairs uniquely identified as hits by TR-FRET and NetMHC, respectively, demonstrating that each approach has the ability to uniquely identify potential neoepitopes (FIG. 13D). The final neoepitope-HLA pair identified by untargeted MS was derived from TP53 R175H (HMTEVVRHC; SEQ ID NO: 128) and was detected on A*02:01. This neoepitope-HLA pair had a RZ-score of 3.9 and a NetMHC % Rank of 3.98 and was not considered a hit by either approach. The detection of this neoepitope-HLA pair demonstrated that even for abundant epitopes, the chosen cutoffs for the TR-FRET assay and presentation prediction algorithms will produce some level of false-negative results. Lastly, of the 22 neoepitope-HLA pairs detected by untargeted analysis, 10 have been previously described in the literature and the remaining 12 neoepitope-HLA pairs were presumed to be novel, based on a search of both Tantigen (Olsen et al., TANTIGEN: a comprehensive database of tumor T cell antigens. Cancer Immunol Immunother. 2017; 66(6): 731-735), CAatlas (Yi et al., caAtlas: An immunopeptidome atlas of human cancer. iScience. 2021; 24(10): 103107), and a cursory search of the literature.

In addition to neoepitopes, untargeted analysis enabled the detection of peptides originating from the non-mutation bearing portions of each 25-mer neoantigen sequence, as well as junction peptides created by sequential 25-mers connected directly or separated by GS linkers within the polyantigen cassette. 27 presented epitopes were detected corresponding to amino acids that flanked but did not contain the mutated residue of the cancer neoantigens. Because these peptides have the same exact sequence as the endogenous version of the protein, it could not be determined if these epitopes were derived from the neoantigen construct or the endogenous genome. However, these peptides still provide information about protein processing, including potential proteasome cleavage sites for these common cancer neoantigens. In addition to these epitopes, 17 and 2 junction peptides were found from the non-linker and linker constructs, respectively (see Table 3). It is reasoned that the lower number of peptides from the linker construct was due to the fact the linker comprised G and S, amino acids which are not typically anchor residues. Lastly, 5 epitopes were identified from antigen sequences included as controls, as well as 13 epitopes derived from BFP (Table 3), supporting that the neoantigen construct was expressed in each of the monoallelic cell lines.

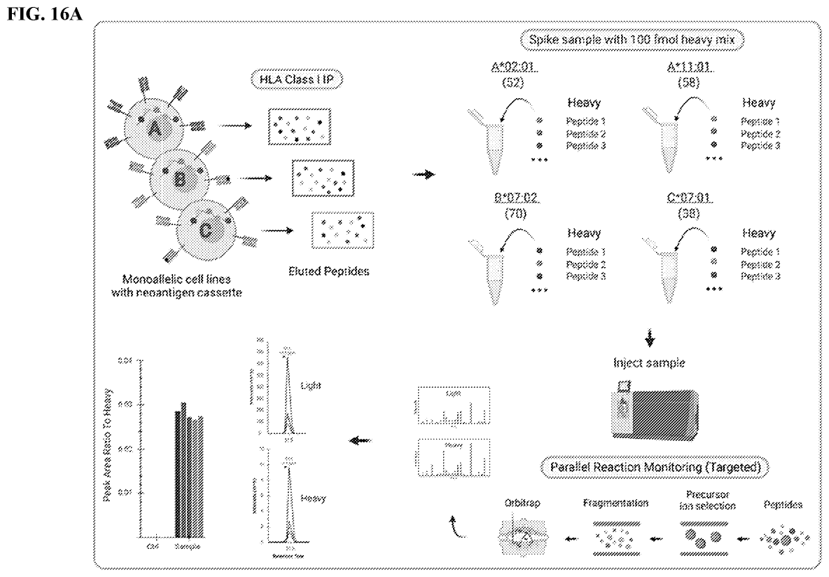

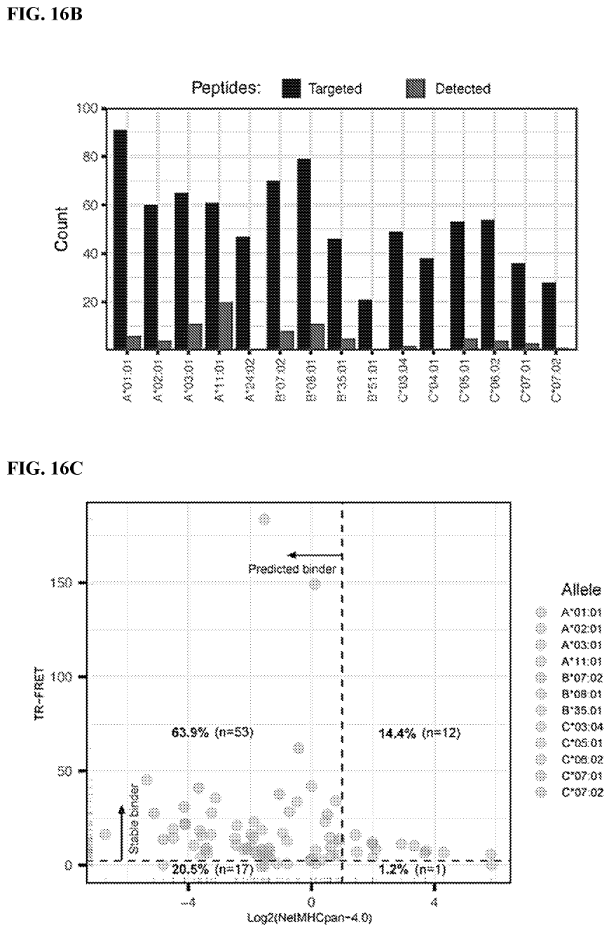

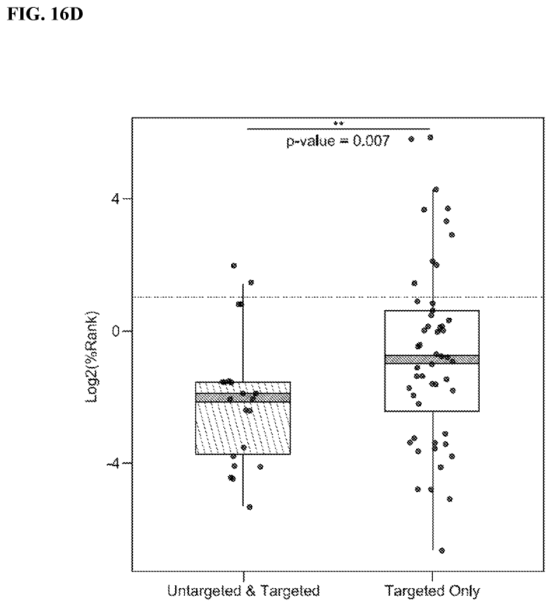

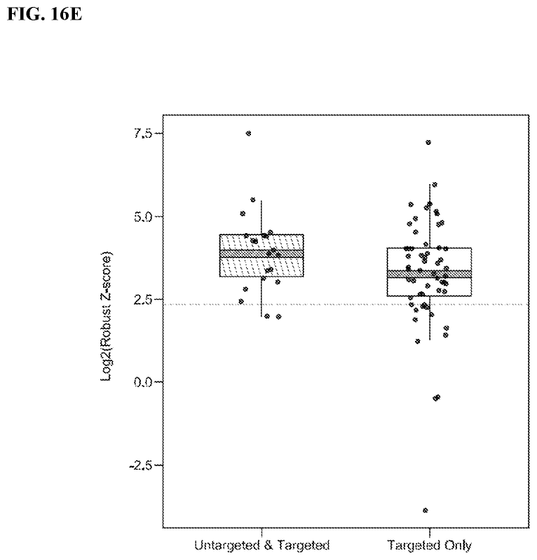

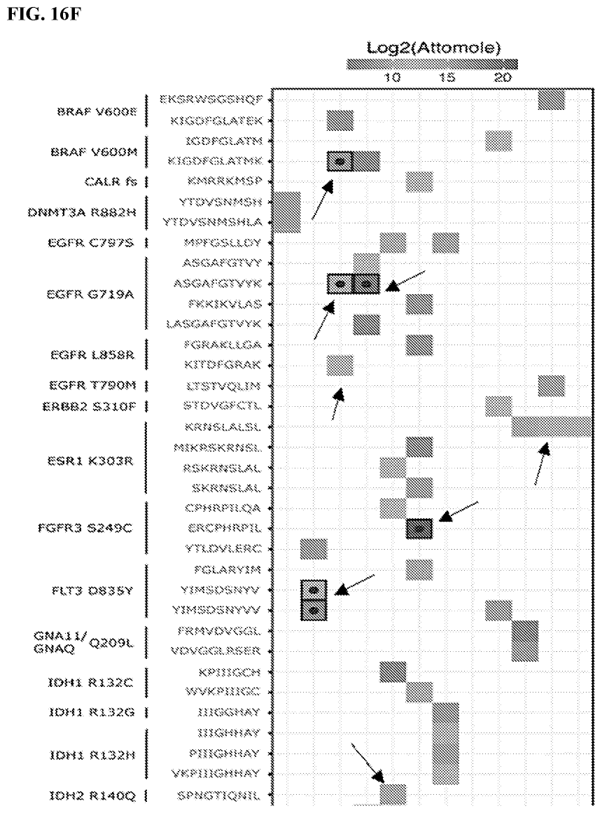

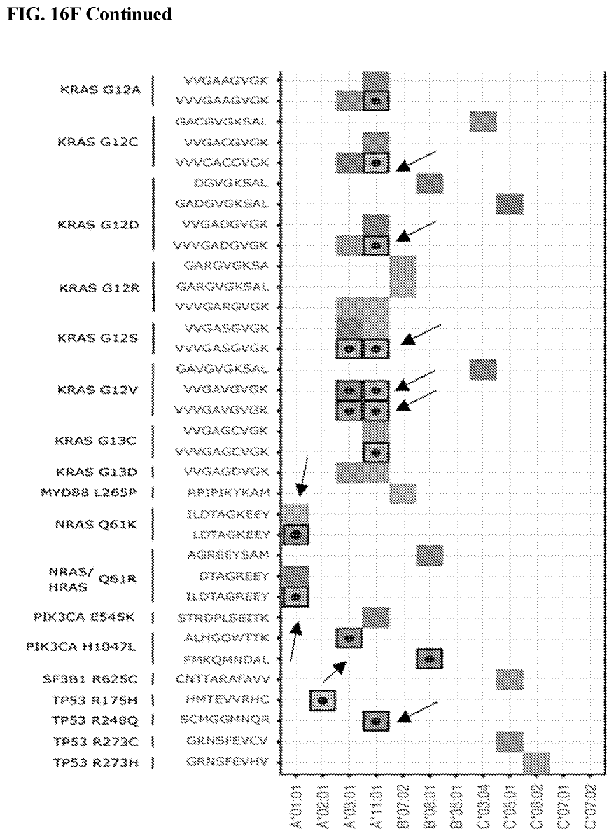

While untargeted analysis enables identification of thousands of peptides, it is challenged by stochastic sampling and limited detection of peptides presented at low copies per cell. Conversely, targeted MS analysis dedicates the entirety of the instrument duty cycle to the analysis of a small number of peptides, enabling detection of peptides presented at low copies per cell and improving data reproducibility. However, targeted approaches require heavy isotope labeled standard peptides and targeted analysis of all potential neoepitopes for the 47 cancer neoantigens within the polyantigen cassette would require 1,748 peptides to be synthesized. Instead, the TR-FRET assay was used as a preliminary screen and synthesized the 397 peptides with a RZ-score≥5 within the TR-FRET (after removing GTF2I L424H from the TR-FRET data). Due to the complementarity of TR-FRET and NetMHC described above, an additional 81 peptides were also synthesized that had a RZ-score<5 but a NetMHC % Rank≤2. Including these peptides allowed to determine if there are neoepitopes predicted by NetMHC that would not have been found within the TR-FRET assay. In total, 479 peptides were divided into allele-specific peptide assays comprising 21 to 88 peptides which were used to quantify neoepitopes across 15 monoallelic cell lines (FIGS. 16A and 16B).

Targeted MS analysis identified 84 neoepitope-HLA pairs across 12 different HLA alleles and 37 shared cancer neoantigens (mutations), representing a four-fold improvement when compared to untargeted MS analysis of the same samples (FIG. 16B). Interestingly, 20 of 84 (˜24%) of all neoepitope-HLA pairs were identified within A*11:01 (FIG. 16B). The high number of A*11:01 epitopes was likely due to the presence of eight distinct KRAS neoantigen sequences within the polyantigen cassette as 14 of 20 A*11:01 specific neoepitopes mapped to KRAS G12X or G13X neoantigens. A similar pattern was observed in A*03:01 where 9 of 14 neoepitopes belonged to KRAS neoantigens. No hits were found for A*24:02, B*51:01, or C*04:01. Following a search of the literature and relevant databases, 24 of the neoepitope-HLA pairs had been described previously and 60 are novel (see Table 6 below).

TABLE 6
Illustrative identified neoepitope-HLA pairs

| | | | Previously |
|---|---|---|---|
| Allele | Neoeptiope Sequence | Neoantigen | Described |
| A*01:01 | YTDVSNMSH (SEQ ID NO: 135) | DNMT3A R882H | NO |
| | YTDVSNMSHLA (SEQ ID NO: 136) | DNMT3A R882H | NO |
| | ILDTAGKEEY (SEQ ID NO: 125) | NRAS Q61K | YES |
| | LDTAGKEEY (SEQ ID NO: 166) | NRAS Q61K | NO |
| | DTAGREEY (SEQ ID NO: 168) | NRAS Q61R/HRAS Q61R | NO |
| | ILDTAGREEY (SEQ ID NO: 126) | NRAS Q61R/HRAS Q61R | YES |
| A*02:01 | YTLDVLERC (SEQ ID NO: 115) | FGFR3 S249C | NO |
| | YIMSDSNYV (SEQ ID NO: 116) | FLT3 D835Y | NO |
| | YIMSDSNYVV (SEQ ID NO: 117) | FLT3 D835Y | NO |
| | HMTEVVRHC (SEQ ID NO: 128) | TP53 R175H | YES |
| A*03:01 | KIGDFGLATEK (SEQ ID NO: 131) | BRAF V600E | NO |
| | KIGDFGLATMK (SEQ ID NO: 133) | BRAF V600M | NO |
| | ASGAFGTVYK (SEQ ID NO: 113) | EGFR G719A | NO |
| | KITDFGRAK (SEQ ID NO: 222) | EGFR L858R | NO |
| | VVVGAAGVGK (SEQ ID NO: 118) | KRAS G12A | NO |
| | VVVGACGVGK (SEQ ID NO: 119) | KRAS G12C | YES |
| | VVVGADGVGK (SEQ ID NO: 120) | KRAS G12D | YES |
| | VVVGARGVGK (SEQ ID NO: 23) | KRAS G12R | YES |
| | VVGASGVGK (SEQ ID NO: 161) | KRAS G12S | NO |
| | VVVGASGVGK (SEQ ID NO: 121) | KRAS G12S | NC |
| | VVGAVGVGK (SEQ ID NO: 122) | KRAS G12V | YES |
| | VVVGAVGVGK (SEQ ID NO: 123) | KRAS G12V | YES |
| | VVGAGDVGK (SEQ ID NO: 164) | KRAS G13D | YES |
| | ALHGGWTTK (SEQ ID NO: 170) | PIK3CA H1047L | YES |
| A*11:01 | KIGDFGLATMK (SEQ ID NO: 133) | BRAF V600M | NO |
| | ASGAFGTVY (SEQ ID NO: 138) | EGFR G719A | NO |
| | ASGAFGTVYK (SEQ ID NO: 113) | EGFR G719A | NO |
| | LASGAFGTVYK (SEQ ID NO: 140) | EGFR G719A | NO |
| | VVGAAGVGK (SEQ ID NO: 155) | KRAS G12A | YES |
| | VVVGAAGVGK (SEQ ID NO: 118) | KRAS G12A | NO |
| | VVGACGVGK (SEQ ID NO: 157) | KRAS G12C | YES |
| | VVVGACGVGK (SEQ ID NO: 119) | KRAS G12C | YES |
| | VVGADGVGK (SEQ ID NO: 160) | KRAS G12D | YES |
| | VVVGADGVGK (SEQ ID NO: 120) | KRAS G12D | YES |
| | VVVGARGVGK (SEQ ID NO: 23) | KRAS G12R | YES |
| | VVGASGVGK (SEQ ID NO: 161) | KRAS G12S | NO |
| | VVVGASGVGK (SEQ ID NO: 121) | KRAS G12S | NO |
| | VVGAVGVGK (SEQ ID NO: 122) | KRAS G12V | YES |
| | VVVGAVGVGK (SEQ ID NO: 123) | KRAS G12V | YES |
| | VVGAGCVGK (SEQ ID NO: 163) | KRAS G13C | NO |
| | VVVGAGCVGK (SEQ ID NO: 124) | KRAS G13C | NO |
| | VVGAGDVGK (SEQ ID NO: 164) | KRAS G13D | NO |
| | STRDPLSEITK (SEQ ID NO: 169) | PIK3CA E545K | NO |
| | SCMGGMNQR (SEQ ID NO: 129) | TP53 R248Q | NO |
| B*07:02 | MPFGSLLDY (SEQ ID NO: 137) | EGFR C797S | NO |
| | RSKRNSLAL (SEQ ID NO: 50) | ESR1 K303R | NO |
| | CPHRPILQA (SEQ ID NO: 144) | FGFR3 S249C | NO |
| | KPIIIGCH (SEQ ID NO: 148) | IDH1 R132C | NO |
| | SPNGTIQNIL (SEQ ID NO: 154) | IDH2 R140Q | YES |
| | GARGVGKSA (SEQ ID NO: 10) | KRAS G12R | YES |
| | GARGVGKSAL (SEQ ID NO: 11) | KRAS G12R | YES |
| | RPIPIKYKAM (SEQ ID NO: 165) | MYD88 L265P | YES |
| B*08:01 | KMRRKMSP (SEQ ID NO: 134) | CALR fs | NO |
| | FKKIKVLAS (SEQ ID NO: 139) | EGFR G719A | NO |
| | FGRAKLLGA (SEQ ID NO: 141) | EGFR L858R | NO |
| | MIKRSKRNSL (SEQ ID NO: 61) | ESR1 K303R | NO |
| | SKRNSLAL (SEQ ID NO: 46) | ESR1 K303R | NO |
| | ERCPHRPIL (SEQ ID NO: 114) | FGFR3 S249C | NO |
| | FGLARYIM (SEQ ID NO: 145) | FLT3 D835Y | NO |
| | WVKPIIIGC (SEQ ID NO: 149) | IDH1 R132C | NO |
| | DGVGKSAL (SEQ ID NO: 158) | KRAS G12D | NO |
| | AGREEYSAM (SEQ ID NO: 167) | NRAS Q61R/HRAS Q61R | NO |
| | FMKQMNDAL (SEQ ID NO: 127) | PIK3CA H1047L | NO |
| B*35:01 | MPFGSLLDY (SEQ ID NO: 137) | EGFR C797S | NO |
| | IIIGGHAY (SEQ ID NO: 150) | IDH1 R132G | NO |
| | IIIGHHAY (SEQ ID NO: 151) | IDH1 R132H | NO |
| | PIIIGHHAY (SEQ ID NO: 174) | IDH1 R132H | NO |
| | VKPIIIGHHAY (SEQ ID NO: 153) | IDH1 R132H | NO |
| C*03:04 | GACGVGKSAL (SEQ ID NO: 156) | KRAS G12C | YES |
| | GAVGVGKSAL (SEQ ID NO: 162) | KRAS G12V | YES |
| C*05:01 | IGDFGLATM (SEQ ID NO: 132) | BRAF V600M | NO |
| | STDVGFCTL (SEQ ID NO: 143) | ERBB2 S310F | NO |
| | YIMSDSNYVV (SEQ ID NO: 117) | FLT3 D835Y | NO |
| | GADGVGKSAL (SEQ ID NO: 159) | KRAS G12D | NO |
| | CNTTARAFAVV (SEQ ID NO: 171) | SF3B1 R625C | NO |
| | GRNSFEVCV (SEQ ID NO: 172) | TP53 R273C | NO |
| C*06:02 | KRNSLALSL (SEQ ID NO: 43) | ESR1 K303R | NO |
| | FRMVDVGGL (SEQ ID NO: 146) | GNAQ Q209L | NO |
| | VDVGGLRSER (SEQ ID NO: 147) | GNAQ Q209L | NO |
| | GRNSFEVHV (SEQ ID NO: 173) | TP53 R273H | NO |
| C*07:01 | EKSRWSGSHQF (SEQ ID NO: 130) | BRAF V600E | NO |
| | LTSTVQLIM (SEQ ID NO: 142) | EGFR T790M | NO |
| | KRNSLALSL (SEQ ID NO: 43) | ESR1 K303R | NO |
| C*07:02 | KRNSLALSL (SEQ ID NO: 43) | ESR1 K303R | NO |

To illustrate the relative value of using the TR-FRET assay and NetMHC as a method to select peptides for targeted MS analysis, RZ-score vs. NetMHC % Rank were plotted for each of the 84 neoepitope-HLA pairs detected by targeted MS (FIG. 16C). This analysis revealed that 53 neoepitopes were stable binders by TR-FRET and predicted to be presented by NetMHC. Additionally, 12 neoepitope-HLA pairs were found as hits in TR-FRET only, while 17 neoepitope-HLA pairs were hits identified only by NetMHC (FIG. 16C). These data demonstrate again that both TR-FRET and NetMHC generally agreed on peptides that would be presented—but each method also identified a unique set of potential neoepitope-HLA combinations.

To illustrate the binding characteristics of the 62 (i.e., the 84 detected epitopes in total minus 22 data-dependent acquisition (DDA) hits) additional neoepitope-HLA pairs identified by targeted analysis, RZ-scores and NetMHC % Rank scores were plotted for peptides observed in both untargeted and targeted analysis and compared them to peptides found only in targeted analysis (FIGS. 16D and 16E). It was found that neoepitope-HLA pairs identified only by targeted analysis had a broader range of NetMHC % Rank scores as compared to neoepitopes also detected in untargeted analysis (FIG. 16D). Furthermore, neoepitope-HLA pairs identified only by targeted analysis had a broader spread of RZ-scores within the TR-FRET assay (FIG. 16E). These results suggest that targeted analysis can identify neoepitopes that are weaker binders as compared to those identified through untargeted analysis.

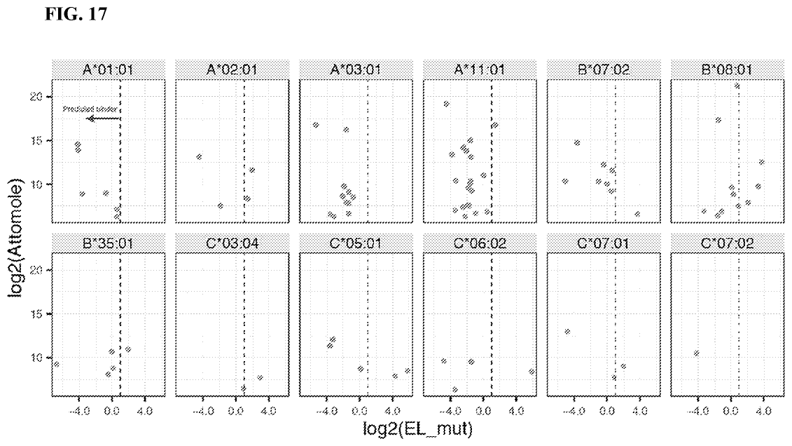

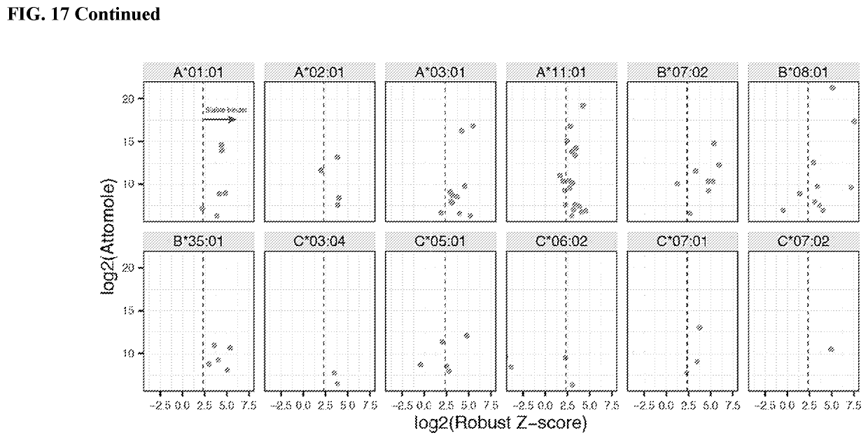

Unlike untargeted approaches, targeted MS permits absolute quantification of peptide presentation enabling comparison of presentation across neoepitopes. Here, the measured amount of neoepitope presentation spanned from 60 amol to 2.5 pmol (FIG. 16F). Unsurprisingly, the peptides detected by untargeted MS generally had higher absolute amounts. For example, both EGFR G719A (ASGAFGTVYK; SEQ ID NO: 113) and FGFR3 S249C (ERCPHRPIL; SEQ ID NO: 114) exhibited the highest absolute abundance—matching the results from the measured intensities from untargeted analysis (FIG. 16F). However, some epitopes such as PIK3CA H1047L (ALHGGWTTK; SEQ ID NO: 170) exhibited high absolute abundance in targeted analysis, but were not detected in untargeted analysis (FIG. 16F). When the absolute amounts of neoepitopes detected were compared to either RZ-score (FIG. 17, the bottom panels) or NetMHC % Rank score (FIG. 17, the top panels) for each allele, no clear correlation could be found. This suggests that each score could be predictive of whether or not a potential neoepitope was presented, but not the absolute amount presented.

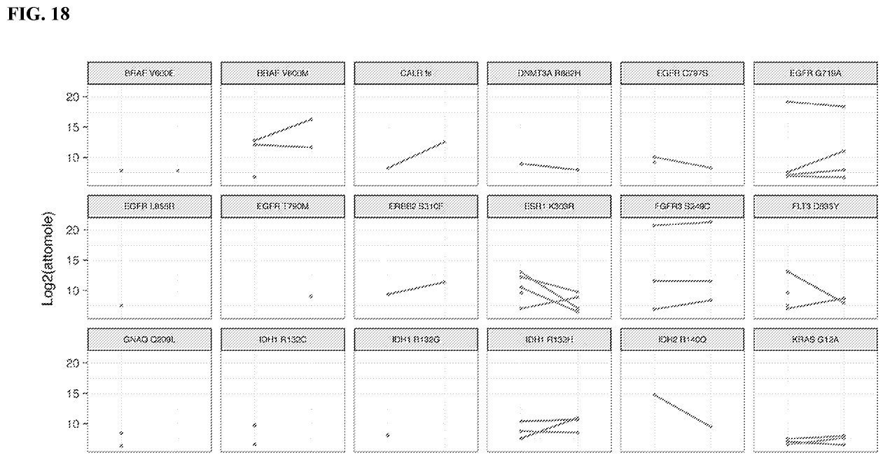

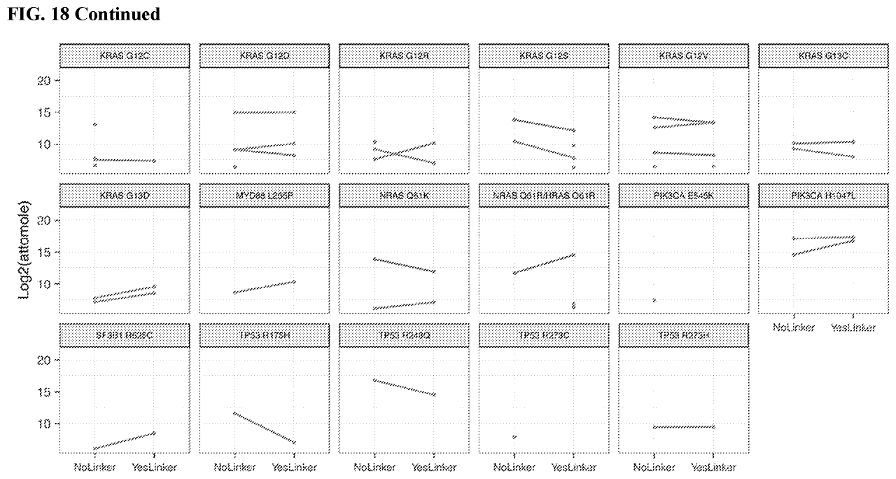

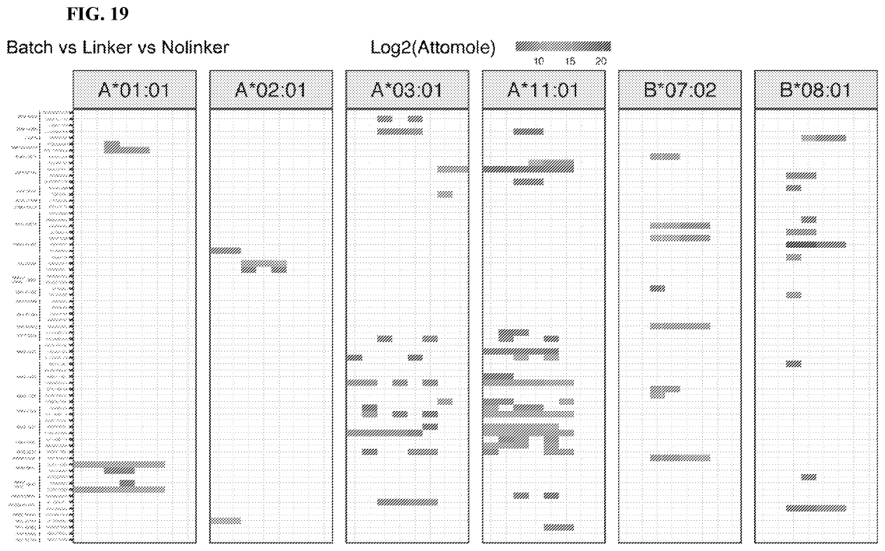

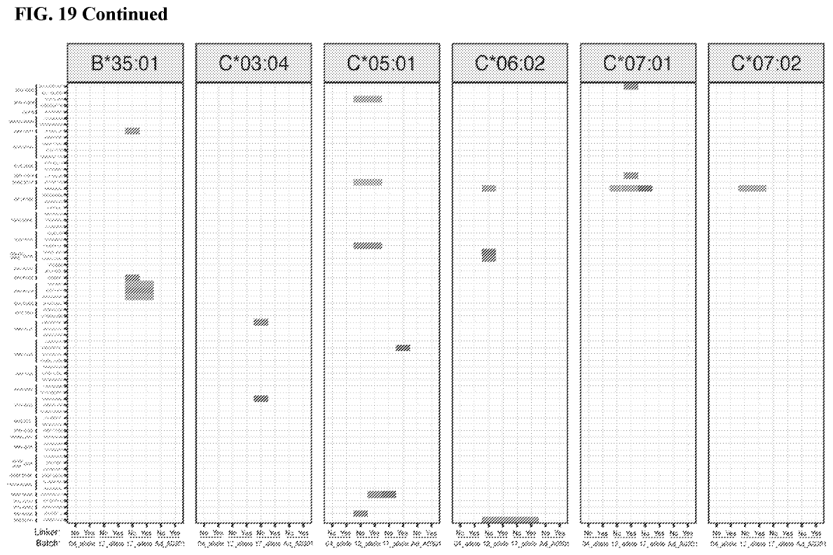

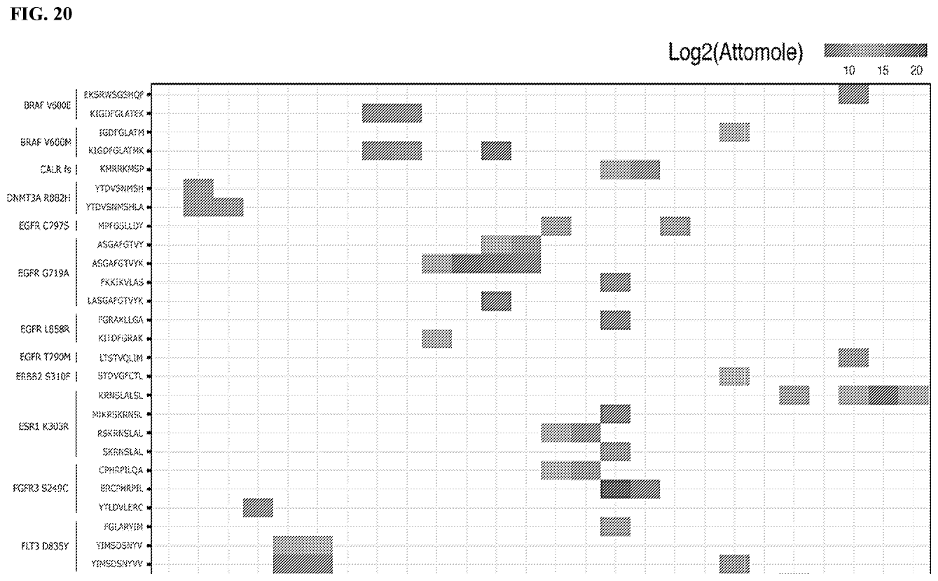

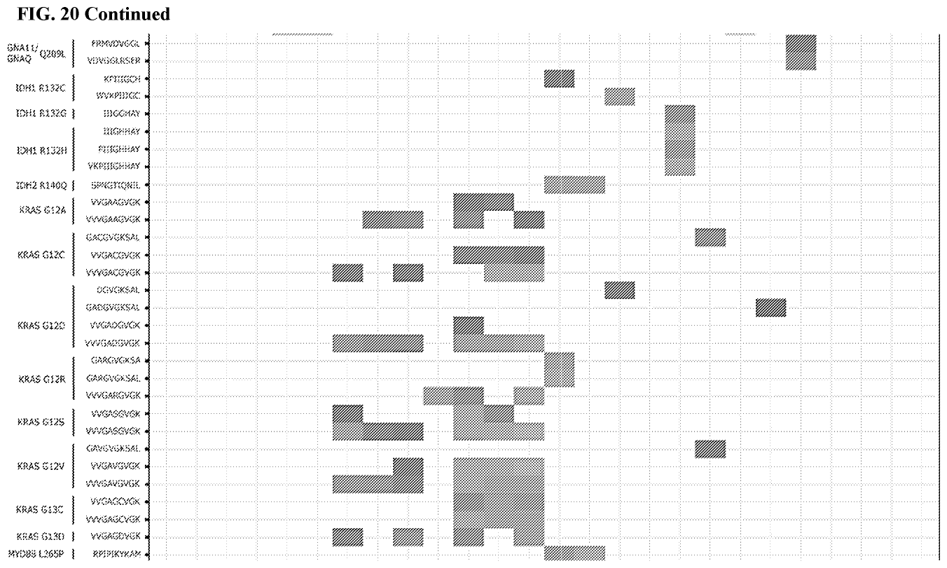

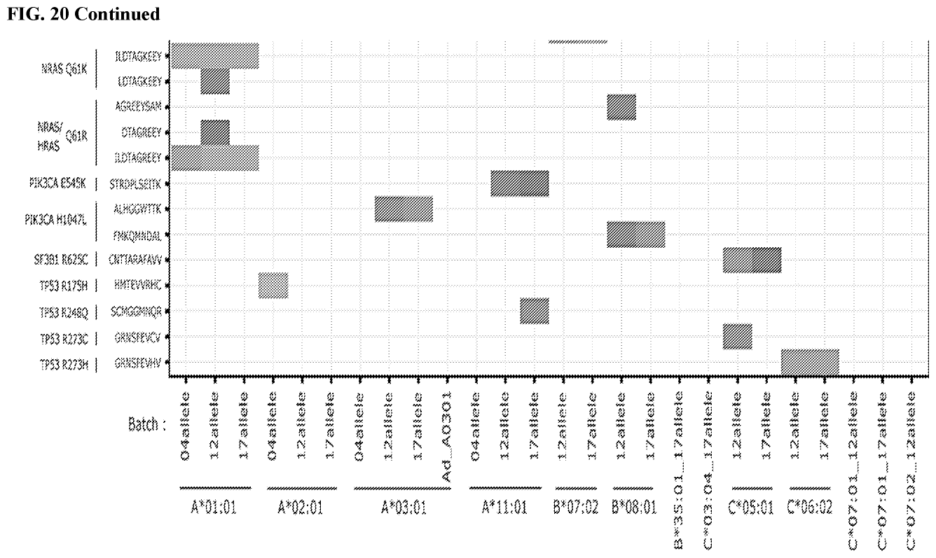

Measurement of absolute levels of neoepitopes also enabled a comparison of presentation from cells containing the linker or no-linker constructs as well as characterization of the reproducibility of measurements across different analysis batches. To illustrate the impact of linkers within the neoantigen construct, plots were prepared for the highest absolute amount of peptide detected for neoepitopes from each cancer neoantigen within monoallelic cell lines containing the linker or no-linker neoantigen constructs (FIGS. 18 and 19). Within this analysis consistent correlations between neoepitope presentation and the presence or absence of linkers in the polyantigen cassette were demonstrated (FIGS. 18 and 19). Lastly, the absolute quantification measurements were collected in 2-3 independent replicates of cell line growth and sample preparation (i.e., HLA-IP and MS analysis) and the absolute measurement of peptide presentation matched well between these replicates (FIG. 20).

### Functional Validation of Novel Tumor Associated Antigen-HLA-I Pairs

A large number of the 84 neoepitope-HLA pairs identified above represent novel candidate neoepitopes. However, presentation of a neoepitope alone does not ensure that it is capable of eliciting a T cell response. To determine whether the identified neoepitopes could be recognized by human T cells, a modified multiplexed TCR discovery method described by Klinger, et al. 2015 PLOS One. 10(10):e0141561 was utilized. Focusing on 2 identified neoeptiope-HLA pairs (Flt3-p.D835Y/HLA-A*02:01,PIK3CA-p.E545K/HLA-A*11:01), briefly, neoepitopes were first allocated to peptide pools in unique combinations before healthy human donor CD8+ T cells were isolated and expanded using autologous monocyte-derived dendritic cells, re-stimulated with the neoepitope pools, sorted for activation marker upregulation, and subjected to TCRβ sequencing. This method was utilized for donors spanning a range of HLA genotypes, enabling the association of TCRs with a variety of peptide-HLA pairs. However, due to the multiallelic nature of donor cells the HLA restriction of identified neoepitopes was not initially disambiguated among the 3-6 donor HLA alleles.

For neoepitopes that elicited a T cell response, associated TCRβ and TCRα sequences were determined using a parallel multiplexed assay (Howie et al., 2015 Sci Transl Med. 7(301): 301ral 31) that enabled construction of paired TCR expression vectors and the selection of candidate neoepitope-specific TCRs. The specificity and potential efficacy of each TCR was then assessed through cellular assays. TCR-encoding in vitro transcribed RNA (ivtRNA) was introduced via electroporation into primary human T cells which were then incubated with either an increasing concentration of the candidate neoepitope in the presence of target cells expressing the predicted HLA allele or monoallelic K562 cells expressing both the predicted HLA allele and neoantigen of interest. These two approaches enabled characterization of TCR potency through activity of an exogenously loaded target cell and the potential for the neoepitope to elicit a T cell response when it is expressed, processed, and presented in a cellular context.

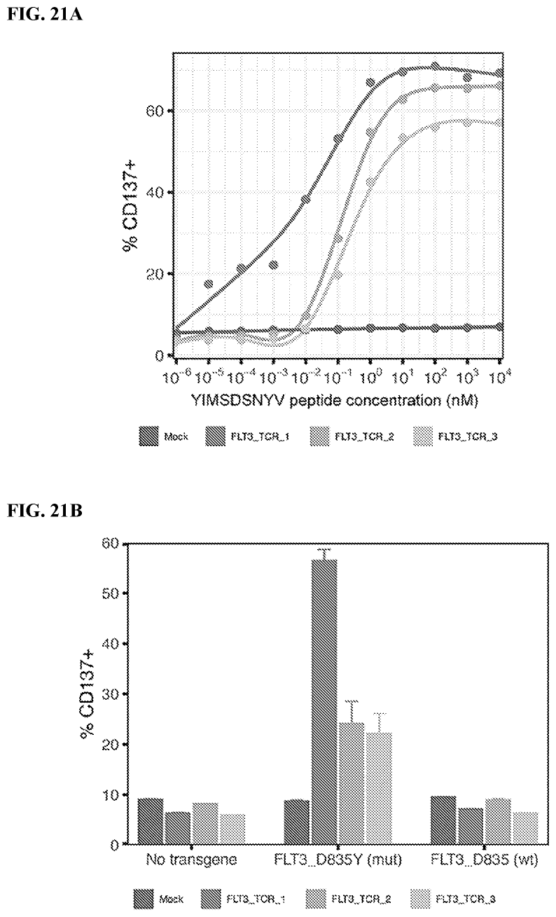

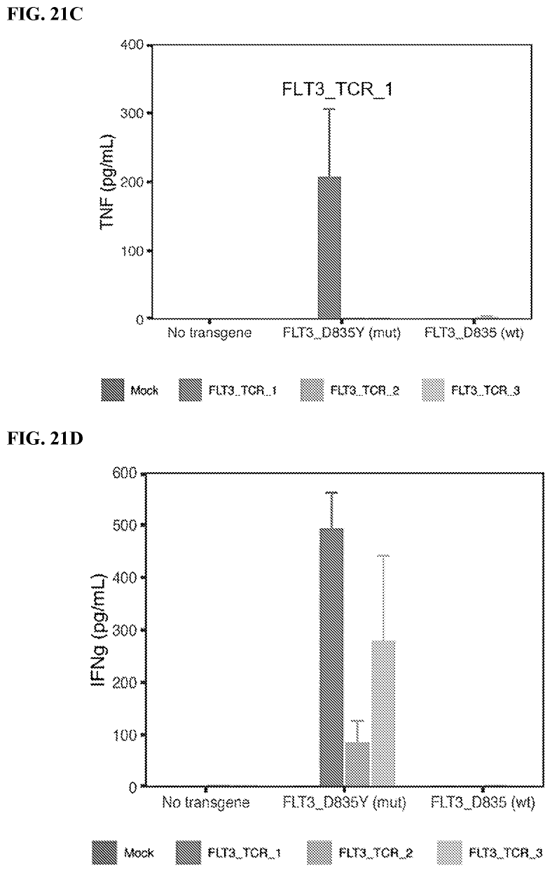

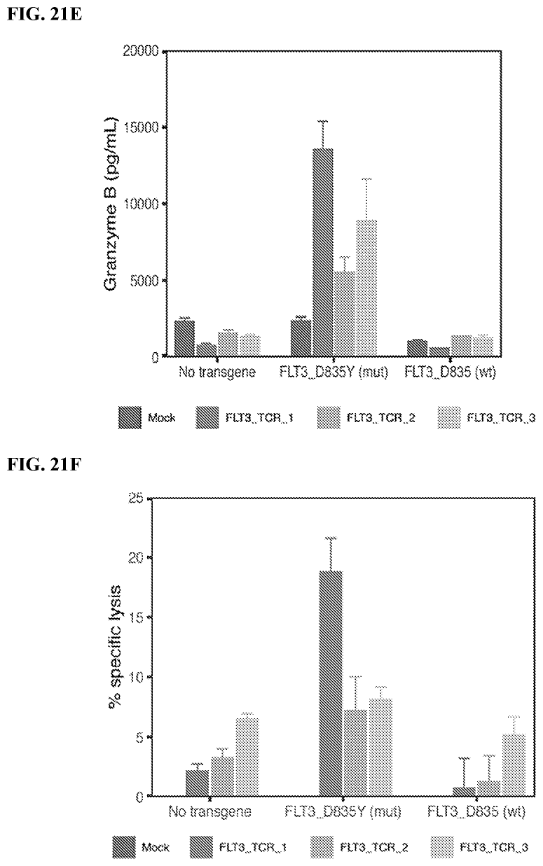

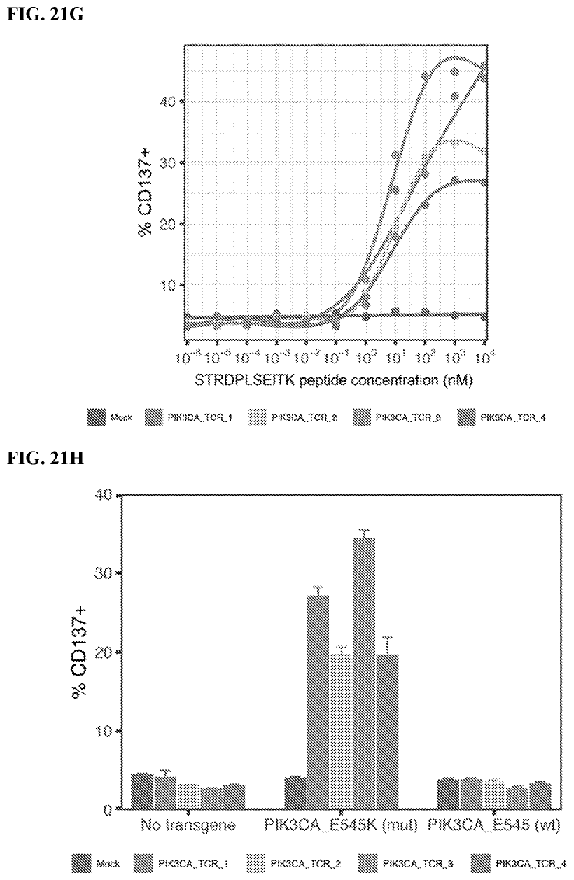

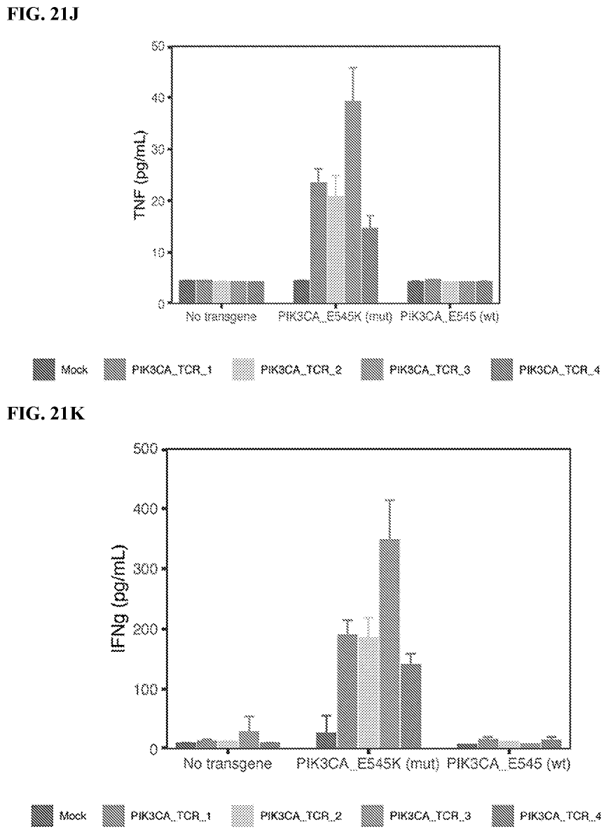

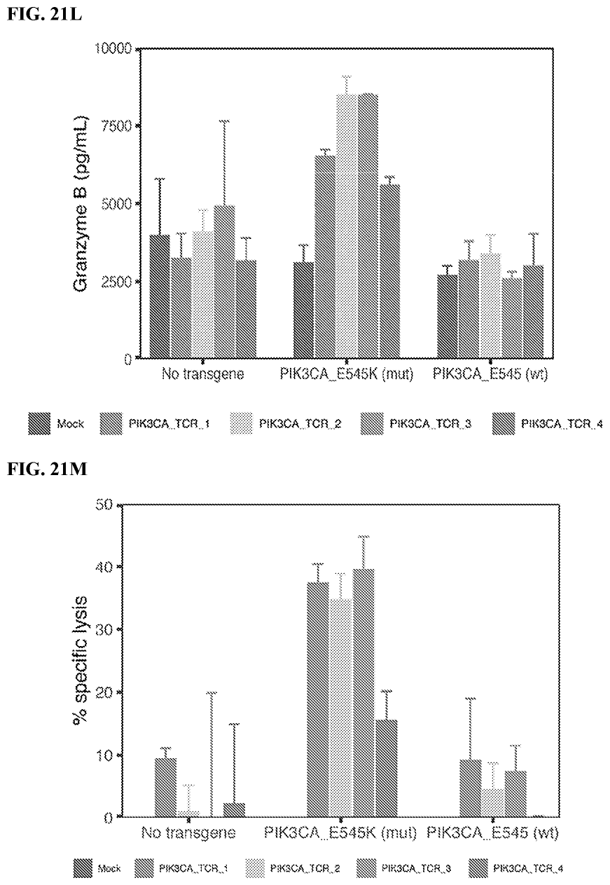

Dose-dependent upregulation of CD137 was found after 12-hour co-culture of primary human CD8+T cells transfected with predicted Flt3-p.D835Y/HLA-A*02:01-specific TCRs, in response to T2 cells (which express low levels of HLA-A*02:01) incubated with the indicated concentrations of exogenously delivered YIMSDSNYV (SEQ ID NO: 116) peptide (FIG. 21A). Furthermore, these T cells were activated by and specifically killed monoallelic A*02:01 K562 cells expressing a mutant FLT3-p.D835Y transgene, but were not activated by and did not kill monoallelic A*02:01 K562 cells expressing a wild type FLT3 transgene (FIGS. 21B-21F). Interestingly, these TCRs appear to be exquisitely specific for the mutant neoepitope, an important characteristic because a similar non-mutant epitope IMSDSNYVV was identified by untargeted analysis in HLA-A*02:01 monoallelic cells. These data suggest a potential utility for these TCRs as a modality to address Flt3-p.D835Y expressing malignancies.

As a second proof of concept, T cells were transfected with predicted PIK3CA-p.E545K/HLA-A*11:01 TCRs and mixed with monoallelic HLA-A*11:01 expressing K562 cells incubated with an increasing concentration of the predicted neoepitope, STRDPLSEITK (SEQ ID NO: 169) (FIG. 21G). Here, TCR transfected T cells demonstrated dose-dependent activation as measured by CD137 expression. Furthermore, these T cells demonstrated higher levels of activation and cell killing when mixed with monoallelic A*11:01 K562 cells expressing a PIK3CA-p.E545K transgene as compared to cells that expressed a wild-type PIK3CA transgene (FIGS. 21H-21M). Mutations that introduce anchor residues are thought to have a high immunogenic potential because the immune system has not built tolerance to a similar WT epitope. For PIK3CA-p.E545K/HLA-A*11:01 the E→K mutation introduces an anchor residue within the context of HLA-A*11:01 and the wild type STRDPLSEITE epitope was not detected in untargeted MS analyses of A*11:01 monoallelic cells. While lack of detection in MS analysis does not demonstrate absence, the WT epitope was also not predicted to bind HLA-A*11:01 by NetMHC. Taken together these data provide a clear mechanism for specificity of PIK3CA-p.E545K TCRs for recognition of mutant PIK3CA as compared to wild-type and support these TCRs as potential therapeutic candidates.

### DISCUSSION

To date, most neoepitope discovery efforts have focused on a limited number of neoantigens, HLA alleles, or both in the search for immunogenic tumor-associated peptides. While a recent report expanded the number of neoantigens and HLA alleles studied at once, these neoepitopes were derived from mutations of the same gene—KRAS (Choi et al., Cell Rep Methods. 2021; 1(5):100084). Here, a multiplexed platform was developed to integrate a high throughput binding assay, computational neoepitope binding prediction, complex cellular engineering of monoallelic cell lines, and targeted mass spectrometry to identify unique tumor-associated neoepitopes that can be presented in the context of specific HLA-I alleles and function as potential targets for neoantigen based cancer immunotherapy. As demonstrated, the workflow leverages the combination of a high throughput biochemical neoepitope-HLA binding assay together with a computational neoepitope-HLA binding algorithm and to enable comprehensive screening of all potential neoepitopes across 47 shared cancer neoantigens and 15 common HLA alleles. The combined analyses yielded a short list of 783 neoepitope-HLA combinations (out of 24,149 total combinations surveyed) that were identified as stable binders and potential candidates for cell surface HLA-I presentation. Separately, a custom engineered monoallelic cell line panel containing a polyantigen cassette encoding all 47 neoantigens was created to increase the breadth of putative neoepitope-HLA combinations presented on the cell surface. With a combination of untargeted and targeted mass spectrometry analysis, 84 unique neoepitope-HLA pairs were identified, deriving from 37 shared cancer neoantigens across 12 of 15 surveyed HLA alleles. To validate the immunogenicity of these unique neoepitope-HLA pairs, and their potential as therapeutic targets, two example combinations (Flt3-p.D835Y/HLA-A*02:01 and PIK3CA-p.E545K/HLA-A*11:01) were selected and cell-based assays were used to evaluate a cohort of neoantigen specific TCRs identified in a separate MIRA workflow. Not only were T cells activated in the presence of corresponding TCR-antigen/HLAI combinations, but several TCRs exhibited mutant peptide-selectivity.

Beyond the analysis provided here, the TR-FRET and MS data represent a valuable resource for future studies of neoepitope presentation. For example, the TR-FRET data could potentially be used as training or benchmarking data for more advanced computational algorithms that predict neoepitope-HLA complex formation. Additionally, raw data were provided for untargeted and targeted MS analysis—enabling future re-analysis with more advanced search algorithms (Vizcaino et al., The Human Immunopeptidome Project: A Roadmap to Predict and Treat Immune Diseases. Mol Cell Proteomics. 2020; 19(1): 31-49), peptide false discovery rate determination (Wilhelm et al., Deep learning boosts sensitivity of mass spectrometry-based immunopeptidomics. Nat Commun. 2021; 12(1):3346), or specific workflows that detect rare events within the antigen presentation pathway (e.g., spliced peptides (Faridi et al., Spliced HLA-bound peptides: a Black Swan event in immunology. Clin Exp Immunol. 2021; 204(2): 179-188)). In addition to the data generated in this study, the monoallelic cell lines expressing the polyantigen cassette also represent an ideal system for future studies characterizing the processing and presentation of clinically actionable shared cancer neoantigens. Collectively, the workflow described herein provides the most comprehensive analyses of the neoepitope landscape performed to-date, and offers critical insight into therapeutic targets for neoepitope based cancer immunotherapies targeting shared neoantigens. One striking finding from this analysis was the relatively few (84 total out of 24,149 initially-screened neoepitope-HLA combinations, or 0.35%) shared neoepitope-HLA pairs presented and, consequently, made available for therapeutic development. Limited presentation of shared cancer neoepitopes could be driven by the diversity of HLA-I peptide binding as neoepitopes for 18 of 37 cancer neoantigens were detected in the context of only one HLA-I allele. For the 19 cancer neoantigens that presented epitopes across multiple HLA-I alleles, 7 were KRAS G12X or G13X mutations. Due to the low incidence of presentation across multiple prevalent HLA-alleles, a broadened use of this platform and additional neoepitope-HLA discovery efforts will be needed to identify the patient populations most likely to benefit from shared neoantigen-specific immunotherapies. For example, the KRAS G12D mutation was reported to be the most frequent in CRC with a frequency of 14.9% (Araujo et al., Molecular profile of KRAS G12C-mutant colorectal and non-small-cell lung cancer. BMC Cancer. 2021; 21(1): 193). Based on the instant analysis, the most prevalent HLA allele presenting a KRAS G12D neoepitope was A*03:01, which on average constitutes an allele frequency of about 14% across patients of Caucasian and European descent. Therefore, for this highly common shared neoantigen within this indication, the maximum patient coverage for European and Caucasian demographic is only ˜2% (mutation frequency X allele frequency). This number decreases further when considering other demographics where the prevalence of HLA-A*03:01 is even lower (African American (1.1%), Chinese (0.2%), Hispanic (1.0%), Southeast Asia (0.75%)). Although there is growing evidence that neoantigen-based therapeutics are highly effective for the treatment of cancer, these collective findings suggest that targeting shared neoantigens will remain challenging.

Although this analysis was comprehensive, it is possible the neoepitope-HLA combinations were missed from either the biochemical assay and/or the prediction algorithms and were not included in the targeted mass spec analysis. Also, presentation was measured in an engineered cell line overexpressing a polyantigen cassette encoding ˜25-mer amino acid fragments spanning the mutated amino acid and it is possible that processing and presentation in this format could be altered compared to full length antigen in a tumor cell, resulting in missed neoepitopes. In view of this potential limitation, neoepitope presentation from cell lines engineered with the full length neoantigen for KRAS G12C, G12D and G12V were evaluated, with an observation of the same neoepitopes presented (FIGS. 12A and 12B). Similar validation may be performed across all 47 neoantigens described herein. Despite these caveats, this is one of the most comprehensive analyses of the neoepitope landscape across the most relevant shared neoantigens and the collective findings yield valuable insight into druggable neoepitope targets for cancer immunotherapy.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

SEQUENCE TABLE
The table below lists exemplary sequences for various molecules.

| SEQ | | | SEQ | | |
|---|---|---|---|---|---|
| ID NO | Identity | Sequence | ID NO | Identity | Sequence |
| 1 | Neoantigen | RGVGKSAL | 2 | Neoantigen | RGVGKSALT |
| | KRAS G12R | | | KRAS G12R | |
| 3 | Neoantigen | RGVGKSALTI | 4 | Neoantigen | RGVGKSALTIQ |
| | KRAS G12R | | | KRAS G12R | |
| 5 | Neoantigen | ARGVGKSA | 6 | Neoantigen | ARGVGKSAL |
| | KRAS G12R | | | KRAS G12R | |
| 7 | Neoantigen | ARGVGKSALT | 8 | Neoantigen | ARGVGKSALTI |
| | KRAS G12R | | | KRAS G12R | |
| 9 | Neoantigen | GARGVGKS | 10 | Neoantigen | GARGVGKSA |
| | KRAS G12R | | | KRAS G12R | |
| 11 | Neoantigen | GARGVGKSAL | 12 | Neoantigen | GARGVGKSALT |
| | KRAS G12R | | | KRAS G12R | |
| 13 | Neoantigen | VGARGVGK | 14 | Neoantigen | VGARGVGKS |
| | KRAS G12R | | | KRAS G12R | |
| 15 | Neoantigen | VGARGVGKSA | 16 | Neoantigen | VGARGVGKSAL |
| | KRAS G12R | | | KRAS G12R | |
| 17 | Neoantigen | VVGARGVG | 18 | Neoantigen | VVGARGVGK |
| | KRAS G12R | | | KRAS G12R | |
| 19 | Neoantigen | VVGARGVGKS | 20 | Neoantigen | VVGARGVGKSA |
| | KRAS G12R | | | KRAS G12R | |
| 21 | Neoantigen | VVVGARGV | 22 | Neoantigen | VVVGARGVG |
| | KRAS G12R | | | KRAS G12R | |
| 23 | Neoantigen | VVVGARGVGK | 24 | Neoantigen | VVVGARGVGKS |
| | KRAS G12R | | | KRAS G12R | |
| 25 | Neoantigen | LVVVGARGV | 26 | Neoantigen | LVVVGARGVG |
| | KRAS G12R | | | KRAS G12R | |
| 27 | Neoantigen | LVVVGARGVGK | 28 | Neoantigen | KLVVVGAR |
| | KRAS G12R | | | KRAS G12R | |
| 29 | Neoantigen | KLVVVGARG | 30 | Neoantigen | KLVVVGARGV |
| | KRAS G12R | | | KRAS G12R | |
| 31 | Neoantigen | KLVVVGARGVG | 32 | Neoantigen | YKLVVVGAR |
| | KRAS G12R | | | KRAS G12R | |
| 33 | Neoantigen | YKLVVVGARG | 34 | Neoantigen | YKLVVVGARGV |
| | KRAS G12R | | | KRAS G12R | |
| 35 | Neoantigen | EYKLVVVGAR | 36 | Neoantigen | EYKLVVVGARG |
| | KRAS G12R | | | KRAS G12R | |
| 37 | Neoantigen | TEYKLVVVGAR | 38 | Neoantigen | RNSLALSL |
| | KRAS G12R | | | ESR1 K303R | |
| 39 | Neoantigen | RNSLALSLT | 40 | Neoantigen | RNSLALSLTA |
| | ESR1 K303R | | | ESR1 K303R | |
| 41 | Neoantigen | RNSLALSLTAD | 42 | Neoantigen | KRNSLALS |
| | ESR1 K303R | | | ESR1 K303R | |
| 43 | Neoantigen | KRNSLALSL | 44 | Neoantigen | KRNSLALSLT |
| | ESR1 K303R | | | ESR1 K303R | |
| 45 | Neoantigen | KRNSLALSLTA | 46 | Neoantigen | SKRNSLAL |
| | ESR1 K303R | | | ESR1 K303R | |
| 47 | Neoantigen | SKRNSLALS | 48 | Neoantigen | SKRNSLALSL |
| | ESR1 K303R | | | ESR1 K303R | |
| 49 | Neoantigen | SKRNSLALSLT | 50 | Neoantigen | RSKRNSLAL |
| | ESR1 K303R | | | ESR1 K303R | |
| 51 | Neoantigen | RSKRNSLALS | 52 | Neoantigen | RSKRNSLALSL |
| | ESR1 K303R | | | ESR1 K303R | |
| 53 | Neoantigen | KRSKRNSLA | 54 | Neoantigen | KRSKRNSLAL |
| | ESR1 K303R | | | ESR1 K303R | |
| 55 | Neoantigen | KRSKRNSLALS | 56 | Neoantigen | IKRSKRNS |
| | ESR1 K303R | | | ESR1 K303R | |
| 57 | Neoantigen | IKRSKRNSLA | 58 | Neoantigen | IKRSKRNSLAL |
| | ESR1 K303R | | | ESR1 K303R | |
| 59 | Neoantigen | MIKRSKRN | 60 | Neoantigen | MIKRSKRNS |
| | ESR1 K303R | | | ESR1 K303R | |
| 61 | Neoantigen | MIKRSKRNSL | 62 | Neoantigen | MIKRSKRNSLA |
| | ESR1 K303R | | | ESR1 K303R | |
| 63 | Neoantigen | LMIKRSKR | 64 | Neoantigen | LMIKRSKRN |
| | ESR1 K303R | | | ESR1 K303R | |
| 65 | Neoantigen | LMIKRSKRNS | 66 | Neoantigen | LMIKRSKRNSL |
| | ESR1 K303R | | | ESR1 K303R | |
| 67 | Neoantigen | PLMIKRSKR | 68 | Neoantigen | PLMIKRSKRN |
| | ESR1 K303R | | | ESR1 K303R | |
| 69 | Neoantigen | PLMIKRSKRNS | 70 | Neoantigen | SPLMIKRSKR |
| | ESR1 K303R | | | ESR1 K303R | |
| 71 | Neoantigen | SPLMIKRSKRN | 72 | Neoantigen | PSPLMIKRSKR |
| | ESR1 K303R | | | ESR1 K303R | |
| 73 | HLA-C*04:01- | TGGCCCGGCCGCG | 74 | HLA-C*04:01- | TTCATCGCAGT |
| | specific | GGGAGCCCCGCTT | | specific | GGGCTACG |
| | sgRNA (long) | CATCGCAGTGGGC | | sgRNA target | |
| | | TACGTGGACGA | | sequence | |
| 75 | shared | ELAGIGILTV | 76 | shared | NLVPMVATV |
| | neoantigen | | | neoantigen | |
| | No. 1 | | | No. 2 | |
| 77 | shared | ATVQGQNLK | 78 | shared | ELAGIGILT |
| | neoantigen | | | neoantigen | |
| | No. 3 | | | No. 4 | |
| 79 | shared | VLEETSVML | 80 | shared | FLYGSKTFI |
| | neoantigen | | | neoantigen | |
| | No. 5 | | | No. 6 | |
| 81 | shared | GTVDNHHFK | 82 | shared | TSNGPVMQK |
| | neoantigen | | | neoantigen | |
| | No. 7 | | | No. 8 | |
| 83 | shared | IYNVKIRGVNF | 84 | shared | VKIRGVNF |
| | neoantigen | | | neoantigen | |
| | No. 9 | | | No. 10 | |
| 85 | shared | KPYEGTQTM | 86 | shared | GPVMQKKTL |
| | neoantigen | | | neoantigen | |
| | No. 11 | | | No. 12 | |
| 87 | shared | YRLERIKEA | 88 | shared | ATSFLYGSK |
| | neoantigen | | | neoantigen | |
| | No. 13 | | | No. 14 | |
| 89 | shared | GSHLIANAK | 90 | shared | KPYEGTQT |
| | neoantigen | | | neoantigen | |
| | No. 15 | | | No. 16 | |
| 91 | shared | GTVDNHHF | 92 | shared | MEGTVDNHHF |
| | neoantigen | | | neoantigen | |
| | No. 17 | | | No. 18 | |
| 93 | shared | KMDWIFHTA | 94 | shared | ATDFVKLKK |
| | neoantigen | | | neoantigen | |
| | No. 19 | | | No. 20 | |
| 95 | shared | RATDFVKLK | 96 | shared | SYVKVLHSI |
| | neoantigen | | | neoantigen | |
| | No. 21 | | | No. 22 | |
| 97 | shared | SPARPGKVV | 98 | shared | DQYMRTIL |
| | neoantigen | | | neoantigen | |
| | No. 23 | | | No. 24 | |
| 99 | shared | VTDLTVKI | 100 | shared | RATDFVRLV |
| | neoantigen | | | neoantigen | |
| | No. 25 | | | No. 26 | |
| 101 | shared | LTIQLIQEQLK | 102 | shared | LTIQLIQNK |
| | neoantigen | | | neoantigen | |
| | No. 27 | | | No. 28 | |
| 103 | shared | AMTEYKLVVV | 104 | shared | LLHRGNYMC |
| | neoantigen | | | neoantigen | |
| | No. 29 | | | No. 30 | |
| 105 | shared | PARPGKVV | 106 | shared | YLLHRGNYM |
| | neoantigen | | | neoantigen | |
| | No. 31 | | | No. 32 | |
| 107 | shared | YVKVLHSI | 108 | shared | YVVRGNAL |
| | neoantigen | | | neoantigen | |
| | No. 33 | | | No. 34 | |
| 109 | shared | SGSGSLSHK | 110 | shared | GSGGEALEY |
| | neoantigen | | | neoantigen | |
| | No. 35 | | | No. 36 | |
| 111 | shared | FPNHVAAIH | 112 | Neoantigen | KIGDFGLATM |
| | neoantigen | | | BRAF | |
| | No. 37 | | | | |
| | | | | V600M | |
| 113 | Neoantigen | ASGAFGTVYK | 114 | Neoantigen | ERCPHRPIL |
| | EGFR G719A | | | FGFR3 | |
| | | | | S249C | |
| 115 | Neoantigen | YTLDVLERC | 116 | Neoantigen | YIMSDSNYV |
| | FGFR3 S249C | | | FLT3 D835Y | |
| 117 | Neoantigen | YIMSDSNYVV | 118 | Neoantigen | VVVGAAGVGK |
| | FLT3 D835Y | | | KRAS G12A | |
| 119 | Neoantigen | VVVGACGVGK | 120 | Neoantigen | VVVGADGVGK |
| | KRAS G12C | | | KRAS G12D | |
| 121 | Neoantigen | VVVGASGVGK | 122 | Neoantigen | VVGAVGVGK |
| | KRAS G12S | | | KRAS G12V | |
| 123 | Neoantigen | VVVGAVGVGK | 124 | Neoantigen | VVVGAGCVGK |
| | KRAS G12V | | | KRAS G13C | |
| 125 | Neoantigen | ILDTAGKEEY | 126 | Neoantigen | ILDTAGREEY |
| | NRAS Q61K | | | NRAS | |
| | | | | Q61R/HRAS | |
| | | | | Q61R | |
| 127 | Neoantigen | FMKQMNDAL | 128 | Neoantigen | HMTEVVRHC |
| | PIK3CA | | | TP53 R175H | |
| | H1047L | | | | |
| 129 | Neoantigen | SCMGGMNQR | 130 | Neoantigen | EKSRWSGSHQF |
| | TP53 R248Q | | | BRAF V600E | |
| 131 | Neoantigen | KIGDFGLATEK | 132 | Neoantigen | IGDFGLATM |
| | BRAF V600E | | | BRAF | |
| | | | | V600M | |
| 133 | Neoantigen | KIGDFGLATMK | 134 | Neoantigen | KMRRKMSP |
| | BRAF V600M | | | CALR fs | |
| 135 | Neoantigen | YTDVSNMSH | 136 | Neoantigen | YTDVSNMSHLA |
| | DNMT3A | | | DNMT3A | |
| | R882H | | | R882H | |
| 137 | Neoantigen | MPFGSLLDY | 138 | Neoantigen | ASGAFGTVY |
| | EGFR C797S | | | EGFR G719A | |
| 139 | Neoantigen | FKKIKVLAS | 140 | Neoantigen | LASGAFGTVYK |
| | EGFR G719A | | | EGFR G719A | |
| 141 | Neoantigen | FGRAKLLGA | 142 | Neoantigen | LTSTVQLIM |
| | EGFR L858R | | | EGFR T790M | |
| 143 | Neoantigen | STDVGFCTL | 144 | Neoantigen | CPHRPILQA |
| | ERBB2 S310F | | | FGFR3 | |
| | | | | S249C | |
| 145 | Neoantigen | FGLARYIM | 146 | Neoantigen | FRMVDVGGL |
| | FLT3 D835Y | | | GNAQ | |
| | | | | Q209L | |
| 147 | Neoantigen | VDVGGLRSER | 148 | Neoantigen | KPIIIGCH |
| | GNAQ Q209L | | | IDH1 R132C | |
| 149 | Neoantigen | WVKPIIIGC | 150 | Neoantigen | IIIGGHAY |
| | IDH1 R132C | | | IDH1 R132G | |
| 151 | Neoantigen | IIIGHHAY | 152 | Neoantigen | PIIIGGHAY |
| | IDH1 R132H | | | IDH1 R132H | |
| 153 | Neoantigen | VKPIIIGHHAY | 154 | Neoantigen | SPNGTIQNIL |
| | IDH1 R132H | | | IDH2 R140Q | |
| 155 | Neoantigen | VVGAAGVGK | 156 | Neoantigen | GACGVGKSAL |
| | KRAS G12A | | | KRAS G12C | |
| 157 | Neoantigen | VVGACGVGK | 158 | Neoantigen | DGVGKSAL |
| | KRAS G12C | | | KRAS G12D | |
| 159 | Neoantigen | GADGVGKSAL | 160 | Neoantigen | VVGADGVGK |
| | KRAS G12D | | | KRAS G12D | |
| 161 | Neoantigen | VVGASGVGK | 162 | Neoantigen | GAVGVGKSAL |
| | KRAS G12S | | | KRAS G12V | |
| 163 | Neoantigen | VVGAGCVGK | 164 | Neoantigen | VVGAGDVGK |
| | KRAS G13C | | | KRAS G13D | |
| 165 | Neoantigen | RPIPIKYKAM | 166 | Neoantigen | LDTAGKEEY |
| | MYD88 L265P | | | NRAS Q61K | |
| 167 | Neoantigen | AGREEYSAM | 168 | Neoantigen | DTAGREEY |
| | NRAS | | | NRAS | |
| | Q61R/HRAS Q61R | | | Q61R/HRAS | |
| | | | | Q61R | |
| 169 | Neoantigen | STRDPLSEITK | 170 | Neoantigen | ALHGGWTTK |
| | PIK3CA | | | PIK3CA | |
| | E545K | | | H1047L | |
| 171 | Neoantigen | CNTTARAFAVV | 172 | Neoantigen | GRNSFEVCV |
| | SF3B1 R625C | | | TP53 R273C | |
| 173 | Neoantigen | GRNSFEVHV | 174 | Neoantigen | PIIIGHHAY |
| | TP53 R273H | | | IDH1 R132H | |
| 175 | TOTAL KRAS | SFEDIHHYR | 176 | Neoantigen | LVVVGACGVGK |
| | | | | KRAS G12C | |
| 177 | Neoantigen | LVVVGADGVGK | 178 | Neoantigen | LVVVGAVGVGK |
| | KRAS G12D | | | KRAS G12V | |
| 179 | KRAS G12 | VVGAGGVGK | 180 | KRAS G12 | VVVGAGGVGK |
| | wild type | | | wild type | |
| 181 | | DARHGGWTT | 182 | | MKQMNDAR |
| 183 | | KICDFGLARY | 184 | | VGGLRSERRKW |
| 185 | | DILDTAGKEEY | 186 | | SKITEQEK |
| 187 | | ATMKSRWSG | 188 | | LSEITKQEK |
| 189 | | MNDARHGGWT | 190 | | KQMNDARH |
| 191 | Neoantigen | KLVVVGAVGV | 192 | Amplicon | CTCCCAGAGCCAC |
| | KRAS G12V | | | Primers: | |
| | | | | Fwd- | |
| | | | | | CGTTACAC |
| 193 | Amplicon | GACTTAAC | 194 | sequencing | CTGGTTGCAGGCG |
| | Primers: Rev- | GCGTC | | primer | |
| | | CTGGTTGC | | | TTTAGCGT |
| 195 | Neoantigen | RATDFVR | 196 | Neoantigen | KMDWIFHDL |
| | IDH1 R132G- | | | PIK3CA | |
| | IDH2 R140Q | | | H1047R-BRAF | |
| | | | | V600E | |
| 197 | Neoantigen | FVKMTEY | 198 | Neoantigen | QEFVKM |
| | JAK V617F- | | | JAK V617F- | |
| | KRAS G13D | | | KRAS G13D | |
| 199 | Neoantigen | QEFVKMT | 200 | Neoantigen | GLQEFC |
| | JAK V617F- | | | Influenza M- | |
| | KRAS G13D | | | IE1 | |
| 201 | Neoantigen | VKVLHSI | 202 | EGFR L858R-EGFR | HASTVQLIT |
| | Mart1-MAGE | | | | |
| | A3 | | | C797S | |
| 203 | EGFR T79M- | YVRGSGSG | 204 | IFWLQW-Linker | IFWLQEGS |
| | Linker | SGSGSL | | | |
| 205 | Linker- | SGSGSLSHK | 206 | Linker- | GSGGEALEY |
| | JAK2 V617F | | | PIK3CA H1047R | |
| 207 | Linker- | GSSGGGSSG | 208 | BFP epitope | YVEQHEVAVARY |
| | KRAS G12C | MTEY | | | |
| 209 | BFP epitope | QHEVAVARY | 210 | MAGE A3 epitope | ALVETSYVK |
| 211 | NYESO1 epitope | FLPVFLAQP | 212 | Influenza M | ILSPLTK |
| | | | | epitope | |
| 213 | NYESO1 epitope | LMWITQV | 214 | Viral epitope | SLLMWITQV |
| 215 | IE-1 epitope | SVMLAKRPLITK | 216 | MAGE A3 epitope | TSYVKVL |
| 217 | BFP epitope | FLYGSKT | 218 | BFP epitope | SFLYGSK |
| 219 | BFP epitope | LYGSKTF | 220 | BFP epitope | KPYEGTQ |
| 221 | BFP epitope | YGSKTFI | 222 | EGFR L858R | KITDFGRAK |

What is claimed is:

1. A method of producing a monoallelic MHC-expressing cell line, comprising:
i) obtaining a cell that does not express an endogenous MHC allele;
ii) introducing into the cell a polynucleotide encoding an exogenous MHC allele polypeptide, such that the exogenous MHC allele polypeptide is expressed by the cell to create a monoallelic MHC-expressing cell; and
iii) expanding the monoallelic MHC-expressing cell under conditions to obtain a monoallelic MHC-expressing cell line.

2. The method of claim 1, wherein the cell in step i) was genetically modified to mutate or delete an endogenous MHC allele.

3. The method of claim 1, wherein step i) comprises genetically modifying a cell to mutate or delete an endogenous MHC allele.

4. The method of any one of claims 1 to 3, wherein the MHC allele is a MHCI allele.

5. The method of any one of claims 1 to 4, wherein the monoallelic MHC-expressing cell line expresses β2-microglobulin (B2M).

6. The method of claim 4, wherein the MHCI allele is encoded by any one of the following loci: HLA-A, HLA-B, and HLA-C.

7. The method of any one of claims 4 to 6, wherein the MHCI allele is selected from A*01.01, A*02.01, A*03.01, A*11.01, A*24.02, B*07.02, B*08.01, B*35.01, B*44.02, B*51.01, C*03.04, C*04.01, C*05.01, C*06.02, C*07.01, C*07.02, and C*08.02.

8. The method of any one of claims 1 to 7, further comprising introducing a polynucleotide cassette encoding a plurality of neoantigen-associated peptides into the monoallelic MHC-expressing cell line.

9. The method of claim 8, wherein the plurality of neoantigen-associated peptides are expressed in the monoallelic MHC-expressing cell line and are cleaved into a plurality of neoepitopes, wherein at least one neoepitope specifically binds to the exogenous MHC polypeptide.

10. The method of claim 9, wherein the at least one neoepitope is 8, 9, 10, 11, 12 or 13 amino acids in length.

11. The method of any one of claims 8 to 10, wherein the neoantigen-associated peptides were identified by bioinformatics and/or a clinical analysis of tumor mutations.

12. The method of any one of claims 8 to 11, wherein at least one of the neoantigen-associated peptides comprises at least 70% sequence identity to at least one of SEQ ID NOs: 1-72, 75-191, and 195-222.

13. The method of any one of claims 8 to 12, wherein each of the plurality of neoantigen-associated peptides is between about 20 and about 50 amino acids in length.

14. The method of any one of claims 8 to 13, wherein at least one of the neoantigen-associated peptides is selected from the neoantigens listed in the Figures.

15. The method of any one of claims 1 to 14, wherein the cell is an antigen-presenting cell (APC).

16. The method of any one of claims 1 to 14, wherein the cell is a HMy2.C1R cell or a K562 cell.

17. A monoallelic MHC-expressing cell line, produced by the method of any one of claims 1 to 16.

18. A system comprising a plurality of monoallelic MHC-expressing cell lines, wherein each cell line does not express an endogenous MHC allele, and wherein each cell line expresses an exogenous MHC allele, such that each cell line expresses a different exogenous MHC allele.

19. The system of claim 18, wherein each of the monoallelic MHC-expressing cell lines was genetically modified to mutate or delete an endogenous MHC allele.

20. The system of claim 18 or 19, wherein each of the expressed MHC alleles is a MHCI allele.

21. The system of any one of claims 18 to 20, wherein the monoallelic MHC-expressing cell line expresses β2-microglobulin (B2M).

22. The system of claim 20, wherein the MHCI allele is encoded by any one of the following loci: HLA-A, HLA-B, and HLA-C.

23. The system of any one of claims 20 to 22, wherein the MHC allele is selected from A*01.01, A*02.01, A*03.01, A*11.01, A*24.02, B*07.02, B*08.01, B*35.01, B*44.02, B*51.01, C*03.04, C*04.01, C*05.01, C*06.02, C*07.01, C*07.02, and C*08.02.

24. The system of any one of claims 18 to 23, wherein each cell line comprises a polynucleotide cassette encoding a plurality of neoantigen-associated peptides.

25. The system of claim 24, wherein the plurality of neoantigen-associated peptides are expressed in the plurality of monoallelic MHC-expressing cell line and are cleaved into a plurality of neoepitopes, wherein at least one neoepitope specifically binds to the exogenous MHC polypeptide.

26. The system of claim 25, wherein the at least one neoepitope is 8, 9, 10, 11, 12 or 13 amino acids in length.

27. The system of any one of claims 24 to 26, wherein the plurality of neoantigen-associated peptides were identified by bioinformatics and/or a clinical analysis of tumor mutations.

28. The system of any one of claims 24 to 26, wherein at least one of the plurality of neoantigen-associated peptides comprises at least 70% sequence identity to at least one of SEQ ID NOs: 1-72, 75-191, and 195-222.

29. The system of any one of claims 24 to 28, wherein each of the plurality of neoantigen-associated peptides is between about 20 and about 50 amino acids in length.

30. The system of any one of claims 24 to 29, wherein at least one of the plurality of neoantigen-associated peptides is selected from the neoantigens listed in the Figures.

31. The system of any one of claims 18 to 30, wherein each of the monoallelic MHC-expressing cell lines is an antigen-presenting cell (APC).

32. The system of any one of claims 18 to 30, wherein the cell is a HMy2.C1R cell or a K562 cell.

33. An isolated polynucleotide cassette encoding a plurality of neoantigen-associated peptides.

34. The isolated polynucleotide cassette of claim 33, wherein each of the plurality of neoantigen-associated peptides is between about 20 and about 50 amino acids in length.

35. The isolated polynucleotide cassette of claim 33 or 34, wherein the plurality of neoantigen-associated peptides was identified by bioinformatics and/or a clinical analysis of tumor mutations.

36. The isolated polynucleotide cassette of any one of claims 33 to 35, further comprising at least a linker between two neoantigen-associated peptides.

37. The isolated polynucleotide cassette of claim 36, wherein the linker comprises a peptide linker.

38. The isolated polynucleotide cassette of any one of claims 33 to 37, further comprising a promoter capable of initiating translation of the plurality of neoantigen-associated peptides into a single polypeptide in a cell.

39. The isolated polynucleotide cassette of claim 38, wherein the single polypeptide is cleaved into a plurality of neoepitopes in the cell.

40. The isolated polynucleotide cassette of claim 39, wherein the plurality of neoepitopes is presented on the surface of the cell.

41. The isolated polynucleotide cassette of any one of claims 33 to 40, wherein at least one of the plurality of neoantigen-associated peptides comprises at least 70% sequence identity to at least one of SEQ ID NOs: 1-72, 75-191, and 195-222.

42. A method of identifying a neoepitope-MHC binding pair, comprising:
i) providing a monoallelic MHC-expressing cell line comprising cells, wherein each cell expresses a first exogenous MHC and comprises a polynucleotide cassette encoding a plurality of neoantigen-associated peptides;
ii) expressing the plurality of neoantigen-associated peptides in each cell, wherein a plurality of neoepitopes is produced by cleaving the plurality of neoantigen-associated peptides within each cell, such that one or more neoepitope binds to the first exogenous MHC at the cell surface;
iii) eluting the neoepitope from its bound first exogenous MHC at the cell surface; and
iv) identifying the eluted neoepitope from step iii), thereby identifying a neoepitope-MHC binding pair.

43. A method of identifying a neoepitope-MHC binding pair, comprising:
i) providing a monoallelic MHC-expressing cell line comprising cells, wherein each cell expresses a first exogenous MHC;
ii) contacting the cells with a synthesized neoepitope;
iii) eluting peptides bound to the first exogenous MHC at the cell surface; and
iv) identifying the eluted neoepitope from step iii), thereby identifying a neoepitope-MHC binding pair.

44. The method of claim 42, wherein the plurality of neoantigen-associated peptides was determined to bind to one or more MHCs by peptide exchange assay.

45. The method of claim 42, wherein the plurality of neoantigen-associated peptides was identified by bioinformatics and/or a clinical analysis of tumor mutations.

46. The method of any one of claims 42 to 45, wherein at least one of the plurality of neoepitopes is 8, 9, 10, 11, 12 or 13 amino acids in length.

47. The method of any one of claims 42 to 46, wherein at least one of the plurality of neoantigen-associated peptides comprises at least 70% sequence identity to at least one of SEQ ID NOs: 1-72, 75-191, and 195-222.

48. The method of any one of claims 42 to 47, wherein each of the plurality of neoantigen-associated peptides is between about 20 and about 50 amino acids in length.

49. The method of any one of claims 42 to 48, wherein at least one of the plurality of neoantigen-associated peptides is selected from the neoantigens listed in the Figures.

50. The method of any one of claims 42 to 49, wherein the plurality of neoantigen-associated peptides are expressed in a single polypeptide.

51. The method of claim 50, wherein the single polypeptide comprises at least one linker between two neoantigen-associated peptides.

52. The method of claim 43, wherein the synthesized neoepitope is determined to bind to one or more MHCs by peptide exchange assay.

53. The method of any one of claims 42 to 52, wherein steps i) to iv) are repeated in a second monoallelic MHC-expressing cell line comprising cells, wherein each cell expresses a second exogenous MHC allele polypeptide.

54. The method of any one of claims 42 to 53, wherein each cell of the monoallelic MHC-expressing cell lines in step i) is genetically modified to mutate or delete an endogenous MHC allele.

55. The method of any one of claims 42 to 54, wherein step i) comprises genetically modifying one or more cells of the monoallelic MHC-expressing cell line to mutate or delete an endogenous MHC allele.

56. The method of any one of claims 42 to 55, wherein the MHC allele is a MHCI allele.

57. The method of any one of claims 42 to 56, wherein the monoallelic MHC-expressing cell line expresses β2-microglobulin (B2M).

58. The method of claim 56, wherein the MHCI allele is encoded by any one of the following loci: HLA-A, HLA-B, and HLA-C.

59. The method of any one of claims 42 to 58, wherein the MHC allele is selected from A*01.01, A*02.01, A*03.01, A*11.01, A*24.02, B*07.02, B*08.01, B*35.01, B*44.02, B*51.01, C*03.04, C*04.01, C*05.01, C*06.02, C*07.01, C*07.02, and C*08.02.

60. A cancer vaccine comprising an isolated polypeptide or an isolated polynucleotide encoding the polypeptide, wherein the polypeptide comprises a neoepitope in the identified neoepitope-MHC binding pair identified by a method of any one of claims 42 to 59.

61. A method of preparing a T cell expressing a T cell receptor (TCR) and/or a chimeric antigen receptor (CAR), comprising introducing a TCR and/or a CAR into a T cell, wherein the TCR and/or the CAR specifically binds to a neoepitope-MHC complex, formed by a neoepitope-MHC binding pair identified by a method of any one of claims 42 to 59.

62. A recombinant T cell expressing a T cell receptor (TCR) and/or a chimeric antigen receptor (CAR), wherein the TCR and/or the CAR specifically binds to a neoepitope-MHC binding pair comprising:
a neoepitope produced by cleaving a neoantigen-associated peptide expressed by a tumor or cancer; and
a MHC expressed by the tumor or cancer.

63. The recombinant T cell of claim 62, wherein the MHC allele peptide is a MHCI allele polypeptide.

64. The recombinant T cell of claim 63, wherein the monoallelic MHC-expressing cell line expresses β2-microglobulin (B2M).

65. The recombinant T cell of claim 63, wherein the MHCI allele is encoded by any one of the following loci: HLA-A, HLA-B, and HLA-C.

66. The recombinant T cell of any one of claims 62 to 65, wherein the MHC allele peptide is encoded by a MHC allele selected from A*01.01, A*02.01, A*03.01, A*11.01, A*24.02, B*07.02, B*08.01, B*35.01, B*44.02, B*51.01, C*03.04, C*04.01, C*05.01, C*06.02, C*07.01, C*07.02, and C*08.02.

67. The recombinant T cell of any one of claims 62 to 66, wherein the neoantigen-associated peptide was identified by bioinformatics and/or a clinical analysis of tumor mutations.

68. The recombinant T cell of any one of claims 62 to 67, wherein the neoepitope is 8, 9, 10, 11, 12 or 13 amino acids in length.

69. The recombinant T cell of any one of claims 62 to 68, wherein the neoantigen-associated peptide comprises at least 70% sequence identity to at least one of SEQ ID NOs: 1-72, 75-191, and 195-222.

70. The recombinant T cell of any one of claims 62 to 69, wherein the neoantigen-associated peptide is between about 20 and about 50 amino acids in length.

71. A method of selecting a subject having a cancer or tumor for treatment by a T cell expressing a T cell receptor (TCR) and/or a chimeric antigen receptor (CAR), comprising
(i) genotyping the subject to identify a MHC allele expressed by the subject and a neoantigen expressed by the cancer or tumor, wherein the neoantigen can be cleaved by a cell to produce a plurality of neoepitopes, and
(ii) determining whether the expressed MHC is capable of binding one or more of the neoepitopes, wherein, if the identified MHC and a neoepitope of the plurality of the neoepitopes form a neoepitope-MHC binding pair, the subject is determined as being treatable by the T cell,
wherein the TCR and/or the CAR specifically binds to the neoepitope-MHC binding pair.

72. A method of selecting a subject having a cancer or tumor for treatment by a cancer vaccine, comprising
(i) genotyping the subject to identify a MHC allele expressed by the subject and a neoantigen expressed by the cancer or tumor wherein the neoantigen can be cleaved by a cell to produce a plurality of neoepitopes, and
(ii) determining whether the MHC is capable of binding one or more of the neoepitopes,
wherein, if the identified MHC and a neoepitope of the plurality of the neoepitopes form a neoepitope-MHC binding pair, the subject is determined to be treatable by a cancer vaccine comprising the neoepitope or a polynucleotide encoding the neoepitope.

73. A method of treating a subject having a cancer or tumor expressing a neoantigen-associated peptide and a MHC, wherein the MHC is determined to bind to a neoepitope from the neoantigen, the method comprising administering to the subject a therapeutically effective amount of T cells expressing a T cell receptor (TCR) and/or a chimeric antigen receptor (CAR), wherein the TCR and/or the CAR specifically binds to a neoepitope-MHC binding pair comprising the MHC and the neoepitope.

74. A method of treating a subject having a cancer or tumor, comprising
(i) selecting a subject expressing a MHC and having a cancer expressing a neoantigen, wherein the MHC is determined to bind to a neoepitope from the neoantigen, and
(ii) administering to the subject a therapeutically effective amount of T cells expressing a T cell receptor (TCR) and/or a chimeric antigen receptor (CAR),
wherein the TCR and/or the CAR specifically binds to a neoepitope-MHC binding pair comprising the MHC and the neoepitope.

75. A method of treating a subject having a cancer or tumor expressing a neoantigen and a MHC, wherein the MHC is determined to bind to a neoepitope from the neoantigen, the method comprising administering to the subject a therapeutically effective amount of a vaccine comprising the neoepitope or a polynucleotide encoding the neoepitope.

76. The method of any one of claims 71 to 75, wherein the neoepitope is determined to bind to one or more MHCs by peptide exchange assay.

77. The method of any one of claims 71 to 76, wherein the neoantigen was identified by bioinformatics and/or a clinical analysis of tumor mutations.

78. The method of any one of claims 71 to 77, wherein the neoantigen is a neoantigen listed in any one of the Figures.

79. The method of any one of claims 71 to 78, wherein the neoepitope is 8, 9, 10, 11, 12 or 13 amino acids in length.

80. The method of any one of claims 71 to 79, wherein the neoepitope comprises at least 70% sequence identity to at least one of SEQ ID NOs: 1-72, 75-191, and 195-222.

81. The method of any one of claims 71 to 80, wherein the MHC allele is a MHCI allele.

82. The method of claim 81, wherein the monoallelic MHC-expressing cell line expresses β2-microglobulin (B2M).

83. The method of claim 81, wherein the MHCI allele is encoded by any one of the following loci: HLA-A, HLA-B, and HLA-C.

84. The method of any one of claims 71 to 83, wherein the MHC allele is selected from A*01.01, A*02.01, A*03.01, A*11.01, A*24.02, B*07.02, B*08.01, B*35.01, B*44.02, B*51.01, C*03.04, C*04.01, C*05.01, C*06.02, C*07.01, C*07.02, and C*08.02.


SEQUENCE LISTING (222 sequences)

SEQ ID NO: 1 - protein, 8 aa, synthetic construct
RGVGKSAL

SEQ ID NO: 2 - protein, 9 aa, synthetic construct
RGVGKSALT

SEQ ID NO: 3 - protein, 10 aa, synthetic construct
RGVGKSALTI

SEQ ID NO: 4 - protein, 11 aa, synthetic construct
RGVGKSALTIQ

SEQ ID NO: 5 - protein, 8 aa, synthetic construct
ARGVGKSA

SEQ ID NO: 6 - protein, 9 aa, synthetic construct
ARGVGKSAL

SEQ ID NO: 7 - protein, 10 aa, synthetic construct
ARGVGKSALT

SEQ ID NO: 8 - protein, 11 aa, synthetic construct
ARGVGKSALTI

SEQ ID NO: 9 - protein, 8 aa, synthetic construct
GARGVGKS

SEQ ID NO: 10 - protein, 9 aa, synthetic construct
GARGVGKSA

SEQ ID NO: 11 - protein, 10 aa, synthetic construct
GARGVGKSAL

SEQ ID NO: 12 - protein, 11 aa, synthetic construct
GARGVGKSALT

SEQ ID NO: 13 - protein, 8 aa, synthetic construct
VGARGVGK

SEQ ID NO: 14 - protein, 9 aa, synthetic construct
VGARGVGKS

SEQ ID NO: 15 - protein, 10 aa, synthetic construct
VGARGVGKSA

SEQ ID NO: 16 - protein, 11 aa, synthetic construct
VGARGVGKSAL

SEQ ID NO: 17 - protein, 8 aa, synthetic construct
VVGARGVG

SEQ ID NO: 18 - protein, 9 aa, synthetic construct
VVGARGVGK

SEQ ID NO: 19 - protein, 10 aa, synthetic construct
VVGARGVGKS

SEQ ID NO: 20 - protein, 11 aa, synthetic construct
VVGARGVGKSA

SEQ ID NO: 21 - protein, 8 aa, synthetic construct
VVVGARGV

SEQ ID NO: 22 - protein, 9 aa, synthetic construct
VVVGARGVG

SEQ ID NO: 23 - protein, 10 aa, synthetic construct
VVVGARGVGK

SEQ ID NO: 24 - protein, 11 aa, synthetic construct
VVVGARGVGKS

SEQ ID NO: 25 - protein, 9 aa, synthetic construct
LVVVGARGV

SEQ ID NO: 26 - protein, 10 aa, synthetic construct
LVVVGARGVG

SEQ ID NO: 27 - protein, 11 aa, synthetic construct
LVVVGARGVGK

SEQ ID NO: 28 - protein, 8 aa, synthetic construct
KLVVVGAR

SEQ ID NO: 29 - protein, 9 aa, synthetic construct
KLVVVGARG

SEQ ID NO: 30 - protein, 10 aa, synthetic construct
KLVVVGARGV

SEQ ID NO: 31 - protein, 11 aa, synthetic construct
KLVVVGARGVG

SEQ ID NO: 32 - protein, 9 aa, synthetic construct
YKLVVVGAR

SEQ ID NO: 33 - protein, 10 aa, synthetic construct
YKLVVVGARG

SEQ ID NO: 34 - protein, 11 aa, synthetic construct
YKLVVVGARGV

SEQ ID NO: 35 - protein, 10 aa, synthetic construct
EYKLVVVGAR

SEQ ID NO: 36 - protein, 11 aa, synthetic construct
EYKLVVVGARG

SEQ ID NO: 37 - protein, 11 aa, synthetic construct
TEYKLVVVGAR

SEQ ID NO: 38 - protein, 8 aa, synthetic construct
RNSLALSL

SEQ ID NO: 39 - protein, 9 aa, synthetic construct
RNSLALSLT

SEQ ID NO: 40 - protein, 10 aa, synthetic construct
RNSLALSLTA

SEQ ID NO: 41 - protein, 11 aa, synthetic construct
RNSLALSLTAD

SEQ ID NO: 42 - protein, 8 aa, synthetic construct
KRNSLALS

SEQ ID NO: 43 - protein, 9 aa, synthetic construct
KRNSLALSL

SEQ ID NO: 44 - protein, 10 aa, synthetic construct
KRNSLALSLT

SEQ ID NO: 45 - protein, 11 aa, synthetic construct
KRNSLALSLTA

SEQ ID NO: 46 - protein, 8 aa, synthetic construct
SKRNSLAL

SEQ ID NO: 47 - protein, 9 aa, synthetic construct
SKRNSLALS

SEQ ID NO: 48 - protein, 10 aa, synthetic construct
SKRNSLALSL

SEQ ID NO: 49 - protein, 11 aa, synthetic construct
SKRNSLALSLT

SEQ ID NO: 50 - protein, 9 aa, synthetic construct
RSKRNSLAL

SEQ ID NO: 51 - protein, 10 aa, synthetic construct
RSKRNSLALS

SEQ ID NO: 52 - protein, 11 aa, synthetic construct
RSKRNSLALSL

SEQ ID NO: 53 - protein, 9 aa, synthetic construct
KRSKRNSLA

SEQ ID NO: 54 - protein, 10 aa, synthetic construct
KRSKRNSLAL

SEQ ID NO: 55 - protein, 11 aa, synthetic construct
KRSKRNSLALS

SEQ ID NO: 56 - protein, 8 aa, synthetic construct
IKRSKRNS

SEQ ID NO: 57 - protein, 10 aa, synthetic construct
IKRSKRNSLA

SEQ ID NO: 58 - protein, 11 aa, synthetic construct
IKRSKRNSLAL

SEQ ID NO: 59 - protein, 8 aa, synthetic construct
MIKRSKRN

SEQ ID NO: 60 - protein, 9 aa, synthetic construct
MIKRSKRNS

SEQ ID NO: 61 - protein, 10 aa, synthetic construct
MIKRSKRNSL

SEQ ID NO: 62 - protein, 11 aa, synthetic construct
MIKRSKRNSLA

SEQ ID NO: 63 - protein, 8 aa, synthetic construct
LMIKRSKR

SEQ ID NO: 64 - protein, 9 aa, synthetic construct
LMIKRSKRN

SEQ ID NO: 65 - protein, 10 aa, synthetic construct
LMIKRSKRNS

SEQ ID NO: 66 - protein, 11 aa, synthetic construct
LMIKRSKRNSL

SEQ ID NO: 67 - protein, 9 aa, synthetic construct
PLMIKRSKR

SEQ ID NO: 68 - protein, 10 aa, synthetic construct
PLMIKRSKRN

SEQ ID NO: 69 - protein, 11 aa, synthetic construct
PLMIKRSKRNS

SEQ ID NO: 70 - protein, 10 aa, synthetic construct
SPLMIKRSKR

SEQ ID NO: 71 - protein, 11 aa, synthetic construct
SPLMIKRSKRN

SEQ ID NO: 72 - protein, 11 aa, synthetic construct
PSPLMIKRSKR

SEQ ID NO: 73 - DNA, 50 bp, synthetic construct
  source 1..50: mol_type = other DNA
tggcccggccgcggggagccccgcttcatcgcagtgggctacgtggacga

SEQ ID NO: 74 - DNA, 19 bp, synthetic construct
  source 1..19: mol_type = other DNA
ttcatcgcagtgggctacg

SEQ ID NO: 75 - protein, 10 aa, synthetic construct
ELAGIGILTV

SEQ ID NO: 76 - protein, 9 aa, synthetic construct
NLVPMVATV

SEQ ID NO: 77 - protein, 9 aa, synthetic construct
ATVQGQNLK

SEQ ID NO: 78 - protein, 9 aa, synthetic construct
ELAGIGILT

SEQ ID NO: 79 - protein, 9 aa, synthetic construct
VLEETSVML

SEQ ID NO: 80 - protein, 9 aa, synthetic construct
FLYGSKTFI

SEQ ID NO: 81 - protein, 9 aa, synthetic construct
GTVDNHHFK

SEQ ID NO: 82 - protein, 9 aa, synthetic construct
TSNGPVMQK

SEQ ID NO: 83 - protein, 11 aa, synthetic construct
IYNVKIRGVNF

SEQ ID NO: 84 - protein, 8 aa, synthetic construct
VKIRGVNF

SEQ ID NO: 85 - protein, 9 aa, synthetic construct
KPYEGTQTM

SEQ ID NO: 86 - protein, 9 aa, synthetic construct
GPVMQKKTL

SEQ ID NO: 87 - protein, 9 aa, synthetic construct
YRLERIKEA

SEQ ID NO: 88 - protein, 9 aa, synthetic construct
ATSFLYGSK

SEQ ID NO: 89 - protein, 9 aa, synthetic construct
GSHLIANAK

SEQ ID NO: 90 - protein, 8 aa, synthetic construct
KPYEGTQT

SEQ ID NO: 91 - protein, 8 aa, synthetic construct
GTVDNHHF

SEQ ID NO: 92 - protein, 10 aa, synthetic construct
MEGTVDNHHF

SEQ ID NO: 93 - protein, 9 aa, synthetic construct
KMDWIFHTA

SEQ ID NO: 94 - protein, 9 aa, synthetic construct
ATDFVKLKK

SEQ ID NO: 95 - protein, 9 aa, synthetic construct
RATDFVKLK

SEQ ID NO: 96 - protein, 9 aa, synthetic construct
SYVKVLHSI

SEQ ID NO: 97 - protein, 9 aa, synthetic construct
SPARPGKVV

SEQ ID NO: 98 - protein, 8 aa, synthetic construct
DQYMRTIL

SEQ ID NO: 99 - protein, 8 aa, synthetic construct
VTDLTVKI

SEQ ID NO: 100 - protein, 9 aa, synthetic construct
RATDFVRLV

SEQ ID NO: 101 - protein, 11 aa, synthetic construct
LTIQLIQEQLK

SEQ ID NO: 102 - protein, 9 aa, synthetic construct
LTIQLIQNK

SEQ ID NO: 103 - protein, 10 aa, synthetic construct
AMTEYKLVVV

SEQ ID NO: 104 - protein, 9 aa, synthetic construct
LLHRGNYMC

SEQ ID NO: 105 - protein, 8 aa, synthetic construct
PARPGKVV

SEQ ID NO: 106 - protein, 9 aa, synthetic construct
YLLHRGNYM

SEQ ID NO: 107 - protein, 8 aa, synthetic construct
YVKVLHSI

SEQ ID NO: 108 - protein, 8 aa, synthetic construct
YVVRGNAL

SEQ ID NO: 109 - protein, 9 aa, synthetic construct
SGSGSLSHK

SEQ ID NO: 110 - protein, 9 aa, synthetic construct
GSGGEALEY

SEQ ID NO: 111 - protein, 9 aa, synthetic construct
FPNHVAAIH

SEQ ID NO: 112 - protein, 10 aa, synthetic construct
KIGDFGLATM

SEQ ID NO: 113 - protein, 10 aa, synthetic construct
ASGAFGTVYK

SEQ ID NO: 114 - protein, 9 aa, synthetic construct
ERCPHRPIL

SEQ ID NO: 115 - protein, 9 aa, synthetic construct
YTLDVLERC

SEQ ID NO: 116 - protein, 9 aa, synthetic construct
YIMSDSNYV

SEQ ID NO: 117 - protein, 10 aa, synthetic construct
YIMSDSNYVV

SEQ ID NO: 118 - protein, 10 aa, synthetic construct
VVVGAAGVGK

SEQ ID NO: 119 - protein, 10 aa, synthetic construct
VVVGACGVGK

SEQ ID NO: 120 - protein, 10 aa, synthetic construct
VVVGADGVGK

SEQ ID NO: 121 - protein, 10 aa, synthetic construct
VVVGASGVGK

SEQ ID NO: 122 - protein, 9 aa, synthetic construct
VVGAVGVGK

SEQ ID NO: 123 - protein, 10 aa, synthetic construct
VVVGAVGVGK

SEQ ID NO: 124 - protein, 10 aa, synthetic construct
VVVGAGCVGK

SEQ ID NO: 125 - protein, 10 aa, synthetic construct
ILDTAGKEEY

SEQ ID NO: 126 - protein, 10 aa, synthetic construct
ILDTAGREEY

SEQ ID NO: 127 - protein, 9 aa, synthetic construct
FMKQMNDAL

SEQ ID NO: 128 - protein, 9 aa, synthetic construct
HMTEVVRHC

SEQ ID NO: 129 - protein, 9 aa, synthetic construct
SCMGGMNQR

SEQ ID NO: 130 - protein, 11 aa, synthetic construct
EKSRWSGSHQF

SEQ ID NO: 131 - protein, 11 aa, synthetic construct
KIGDFGLATEK

SEQ ID NO: 132 - protein, 9 aa, synthetic construct
IGDFGLATM

SEQ ID NO: 133 - protein, 11 aa, synthetic construct
KIGDFGLATMK

SEQ ID NO: 134 - protein, 8 aa, synthetic construct
KMRRKMSP

SEQ ID NO: 135 - protein, 9 aa, synthetic construct
YTDVSNMSH

SEQ ID NO: 136 - protein, 11 aa, synthetic construct
YTDVSNMSHLA

SEQ ID NO: 137 - protein, 9 aa, synthetic construct
MPFGSLLDY

SEQ ID NO: 138 - protein, 9 aa, synthetic construct
ASGAFGTVY

SEQ ID NO: 139 - protein, 9 aa, synthetic construct
FKKIKVLAS

SEQ ID NO: 140 - protein, 11 aa, synthetic construct
LASGAFGTVYK

SEQ ID NO: 141 - protein, 9 aa, synthetic construct
FGRAKLLGA

SEQ ID NO: 142 - protein, 9 aa, synthetic construct
LTSTVQLIM

SEQ ID NO: 143 - protein, 9 aa, synthetic construct
STDVGFCTL

SEQ ID NO: 144 - protein, 9 aa, synthetic construct
CPHRPILQA

SEQ ID NO: 145 - protein, 8 aa, synthetic construct
FGLARYIM

SEQ ID NO: 146 - protein, 9 aa, synthetic construct
FRMVDVGGL

SEQ ID NO: 147 - protein, 10 aa, synthetic construct
VDVGGLRSER

SEQ ID NO: 148 - protein, 8 aa, synthetic construct
KPIIIGCH

SEQ ID NO: 149 - protein, 9 aa, synthetic construct
WVKPIIIGC

SEQ ID NO: 150 - protein, 8 aa, synthetic construct
IIIGGHAY

SEQ ID NO: 151 - protein, 8 aa, synthetic construct
IIIGHHAY

SEQ ID NO: 152 - protein, 9 aa, synthetic construct
PIIIGGHAY

SEQ ID NO: 153 - protein, 11 aa, synthetic construct
VKPIIIGHHAY

SEQ ID NO: 154 - protein, 10 aa, synthetic construct
SPNGTIQNIL

SEQ ID NO: 155 - protein, 9 aa, synthetic construct
VVGAAGVGK

SEQ ID NO: 156 - protein, 10 aa, synthetic construct
GACGVGKSAL

SEQ ID NO: 157 - protein, 9 aa, synthetic construct
VVGACGVGK

SEQ ID NO: 158 - protein, 8 aa, synthetic construct
DGVGKSAL

SEQ ID NO: 159 - protein, 10 aa, synthetic construct
GADGVGKSAL

SEQ ID NO: 160 - protein, 9 aa, synthetic construct
VVGADGVGK

SEQ ID NO: 161 - protein, 9 aa, synthetic construct
VVGASGVGK

SEQ ID NO: 162 - protein, 10 aa, synthetic construct
GAVGVGKSAL

SEQ ID NO: 163 - protein, 9 aa, synthetic construct
VVGAGCVGK

SEQ ID NO: 164 - protein, 9 aa, synthetic construct
VVGAGDVGK

SEQ ID NO: 165 - protein, 10 aa, synthetic construct
RPIPIKYKAM

SEQ ID NO: 166 - protein, 9 aa, synthetic construct
LDTAGKEEY

SEQ ID NO: 167 - protein, 9 aa, synthetic construct
AGREEYSAM

SEQ ID NO: 168 - protein, 8 aa, synthetic construct
DTAGREEY

SEQ ID NO: 169 - protein, 11 aa, synthetic construct
STRDPLSEITK

SEQ ID NO: 170 - protein, 9 aa, synthetic construct
ALHGGWTTK

SEQ ID NO: 171 - protein, 11 aa, synthetic construct
CNTTARAFAVV

SEQ ID NO: 172 - protein, 9 aa, synthetic construct
GRNSFEVCV

SEQ ID NO: 173 - protein, 9 aa, synthetic construct
GRNSFEVHV

SEQ ID NO: 174 - protein, 9 aa, synthetic construct
PIIIGHHAY

SEQ ID NO: 175 - protein, 9 aa, synthetic construct
SFEDIHHYR

SEQ ID NO: 176 - protein, 11 aa, synthetic construct
LVVVGACGVGK

SEQ ID NO: 177 - protein, 11 aa, synthetic construct
LVVVGADGVGK

SEQ ID NO: 178 - protein, 11 aa, synthetic construct
LVVVGAVGVGK

SEQ ID NO: 179 - protein, 9 aa, synthetic construct
VVGAGGVGK

SEQ ID NO: 180 - protein, 10 aa, synthetic construct
VVVGAGGVGK

SEQ ID NO: 181 - protein, 9 aa, synthetic construct
DARHGGWTT

SEQ ID NO: 182 - protein, 8 aa, synthetic construct
MKQMNDAR

SEQ ID NO: 183 - protein, 10 aa, synthetic construct
KICDFGLARY

SEQ ID NO: 184 - protein, 11 aa, synthetic construct
VGGLRSERRKW

SEQ ID NO: 185 - protein, 11 aa, synthetic construct
DILDTAGKEEY

SEQ ID NO: 186 - protein, 8 aa, synthetic construct
SKITEQEK

SEQ ID NO: 187 - protein, 9 aa, synthetic construct
ATMKSRWSG

SEQ ID NO: 188 - protein, 9 aa, synthetic construct
LSEITKQEK

SEQ ID NO: 189 - protein, 10 aa, synthetic construct
MNDARHGGWT

SEQ ID NO: 190 - protein, 8 aa, synthetic construct
KQMNDARH

SEQ ID NO: 191 - protein, 10 aa, synthetic construct
KLVVVGAVGV

SEQ ID NO: 192 - DNA, 21 bp, synthetic construct
  source 1..21: mol_type = other DNA
ctcccagagccaccgttacac

SEQ ID NO: 193 - DNA, 21 bp, synthetic construct
  source 1..21: mol_type = other DNA
gacttaacgcgtcctggttgc

SEQ ID NO: 194 - DNA, 21 bp, synthetic construct
  source 1..21: mol_type = other DNA
ctggttgcaggcgtttagcgt

SEQ ID NO: 195 - protein, 7 aa, synthetic construct
RATDFVR

SEQ ID NO: 196 - protein, 9 aa, synthetic construct
KMDWIFHDL

SEQ ID NO: 197 - protein, 7 aa, synthetic construct
FVKMTEY

SEQ ID NO: 198 - protein, 6 aa, synthetic construct
QEFVKM

SEQ ID NO: 199 - protein, 7 aa, synthetic construct
QEFVKMT

SEQ ID NO: 200 - protein, 6 aa, synthetic construct
GLQEFC

SEQ ID NO: 201 - protein, 7 aa, synthetic construct
VKVLHSI

SEQ ID NO: 202 - protein, 9 aa, synthetic construct
HASTVQLIT

SEQ ID NO: 203 - protein, 14 aa, synthetic construct
YVRGSGSGSGSGSL

SEQ ID NO: 204 - protein, 8 aa, synthetic construct
IFWLQEGS

SEQ ID NO: 205 - protein, 9 aa, synthetic construct
SGSGSLSHK

SEQ ID NO: 206 - protein, 9 aa, synthetic construct
GSGGEALEY

SEQ ID NO: 207 - protein, 13 aa, synthetic construct
GSSGGGSSGMTEY

SEQ ID NO: 208 - protein, 12 aa, synthetic construct
YVEQHEVAVARY

SEQ ID NO: 209 - protein, 9 aa, synthetic construct
QHEVAVARY

SEQ ID NO: 210 - protein, 9 aa, synthetic construct
ALVETSYVK

SEQ ID NO: 211 - protein, 9 aa, synthetic construct
FLPVFLAQP

SEQ ID NO: 212 - protein, 7 aa, synthetic construct
ILSPLTK

SEQ ID NO: 213 - protein, 7 aa, synthetic construct
LMWITQV

SEQ ID NO: 214 - protein, 9 aa, synthetic construct
SLLMWITQV

SEQ ID NO: 215 - protein, 12 aa, synthetic construct
SVMLAKRPLITK

SEQ ID NO: 216 - protein, 7 aa, synthetic construct
TSYVKVL

SEQ ID NO: 217 - protein, 7 aa, synthetic construct
FLYGSKT

SEQ ID NO: 218 - protein, 7 aa, synthetic construct
SFLYGSK

SEQ ID NO: 219 - protein, 7 aa, synthetic construct
LYGSKTF

SEQ ID NO: 220 - protein, 7 aa, synthetic construct
KPYEGTQ

SEQ ID NO: 221 - protein, 7 aa, synthetic construct
YGSKTFI

SEQ ID NO: 222 - protein, 9 aa, synthetic construct
KITDFGRAK